(19) United States
(12) Patent Application Publication
(10) Pub. No.: US 2024/0018154 A1
(43) Pub. Date: Jan. 18, 2024

(54) NEW INDAZOLE DERIVATIVES

(71) Applicant: Hoffmann-La Roche Inc., Little Falls, NJ (US)

(72) Inventors: Cosimo DOLENTE, Allschwil (CH); Annick GOERGLER, Colmar (FR); David Stephen HEWINGS, Abingdon (GB); Georg JAESCHKE, Basel (CH); Christa Ulrike OBST-SANDER, Reinach BL (CH); Antonio RICCI, Biel-Benken (CH)

(73) Assignee: Hoffmann-La Roche Inc., Little Falls, NJ (US)

(21) Appl. No.: 18/255,076

(22) PCT Filed: Nov. 29, 2021
(86) PCT No.: PCT/EP2021/083301

§ 371 Date: May 30, 2023

(30) Foreign Application Priority Data
Dec. 1, 2020 (EP) 20210868.4

(51) Int. Cl. C07D 487/04; C07D 519/00

(52) U.S. Cl. CPC C07D 487/04; C07D 519/00

(57) ABSTRACT

The invention relates to a compound of formula (I) or a pharmaceutically acceptable salt thereof, wherein R¹-R⁴ and A are as defined in the description and in the claims. The compound of formula (I) can be used as a medicament.

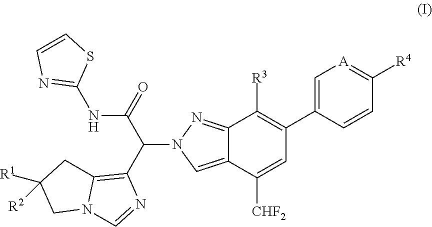

The present invention relates to compounds that are selective allosteric inhibitors of T790M/L858R, T790M/L858R/C797S, L858R and/or L858R/C797S containing EGFR mutants, their manufacture, pharmaceutical compositions containing it and their use as therapeutically active substances.

The invention relates in particular to a novel compound of formula (I)

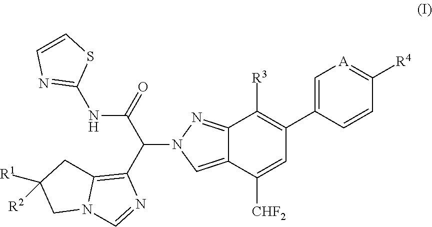

wherein
A is —N— or —CH—;
R¹ and R² are independently selected from halogen and hydrogen;
R³ is alkyl; and
R⁴ is selected from heterocycloalkyl, heterocycloalkylalkoxy, alkylaminoalkyl, dialkylaminoalkyl, hydroxyalkylheterocycloalkylalkoxy, hydroxyalkylheterocycloalkylalkyl, hydroxyheterocycloalkylalkyl, hydroxyheterocycloalkylalkoxy, (alkyl)(halo)heterocycloalkyl, hydroxyheterocycloalkyl, hydroxyalkylheterocycloalkylalkylcycloalkyl, alkylsulfonylheterocycloalkyl, hydroxyheterocycloalkylalkylcycloalkyl, hydroxyalkylheterocycloalkylalkylheterocycloalkyl, hydroxyalkylheterocycloalkyl, haloalkylheterocycloalkyl, (hydroxy)(alkyl)heterocycloalkyl, hydroxycycloalkyl(alkylamino)alkyl, dialkylaminoalkylheterocycloalkyl and heterocycloalkylalkyl;
or a pharmaceutically acceptable salt thereof.

The HER family receptor tyrosine kinases are mediators of cell growth, differentiation and survival. The receptor family includes four distinct members, i.e. epidermal growth factor receptor (EGFR, ErbBl, or HER1) HER2 (ErbB2), HER3 (ErbB3) and HER4 (ErbB4). Upon ligand binding the receptors form homo and heterodimers and subsequent activation of the intrinsic tyrosine kinase activity leads to receptor auto-phosphorylation and the activation of downstream signaling molecules (Yarden, Y., Sliwkowski, MX. Untangling the ErbB signalling network. Nature Review Mol Cell Biol. 2001 February; 2(2): 127-37). De-regulation of EGFR by overexpression or mutation has been implicated in many types of human cancer including colorectal, pancreatic, gliomas, head and neck and lung cancer, in particular non-small cell lung cancer (NSCLC) and several EGFR targeting agents have been developed over the years (Ciardiello, F., and Tortora, G. (2008). EGFR antagonists in cancer treatment. The New England journal of medicine 358, 1160-1174). Erlotinib (Tarceva®), a reversible inhibitor of the EGFR tyrosine kinase was approved in numerous countries for the treatment of recurrent NSCLC.

An impressive single agent activity of EGFR tyrosine kinase inhibitors is observed in a subset of NSCLC patients whose tumors harbor somatic kinase domain mutations, whereas clinical benefit in wild-type EGFR patients is greatly diminished (Paez, J. et al. (2004). EGFR mutations in lung cancer: correlation with clinical response to gefitinib therapy. Science (New York, NY 304, 1497-1500). The most common somatic mutations of EGFR are exon 19 deletions with delta 746 750 the most prevalent mutation and the exon 21 amino acid substitutions with L858R the most frequent mutation (Sharma S V, Bell D W, Settleman J, Haber D A. Epidermal growth factor receptor mutations in lung cancer. Nat Rev Cancer. 2007 March; 7(3): 169-81).

Treatment resistance arises frequently, often due to the secondary T790M mutation within the ATP site of the receptor. Some developed mutant-selective irreversible inhibitors are highly active against the T790M mutant, but their efficacy can be compromised by acquired mutation of C797S, that is the cysteine residue with which they form a key covalent bond (Thress, K. S. et al. Acquired EGFR C797S mutation mediates resistance to AZD9291 in non-small cell lung cancer harboring EGFR T790M. Nat. Med. 21, 560-562 (2015)). C797S mutation was further reported by Wang to be a major mechanism for resistance to T790M-targeting EGFR inhibitors (Wang et al. EGFR C797S mutation mediates resistance to third-generation inhibitors in T790M-positive non-small cell lung cancer, J Hematol Oncol. 2016; 9: 59). Additional mutations that cause resistance to Osimertinib are described by Yang, for example L718Q. (Yang et al, Investigating Novel Resistance Mechanisms to Third-Generation EGFR Tyrosine Kinase Inhibitor Osimertinib in Non-Small Cell Lung Cancer Patients, Clinical Cancer Research, DOI: 10.1158/1078-0432.CCR-17-2310) Lu et al. (Targeting EGFR^{L858R/T790M} and EGFR^{L858R/T790M/C797S} resistance mutations in NSCLC: Current developments in medicinal chemistry, Med Res Rev 2018; 1-32) report in a review article on Targeting EGFR^{L858R/T790M} and EGFR^{L858R/T790M/C797S} resistance mutations in NSCLC treatment.

As most available EGFR tyrosine kinase inhibitors target the ATP-site of the kinase, there is a need for new therapeutic agents that work differently, for example through targeting drug-resistant EGFR mutants.

Recent studies suggest that purposefully targeting allosteric sites might lead to mutant-selective inhibitors (Jia et al. Overcoming EGFR(T790M) and EGFR(C797S) resistance with mutant-selective allosteric inhibitors, June 2016, Nature 534, 129-132) There is therefore an unmet need for the generation of selective molecules that specifically inhibit T790M/L858R, T790M/L858R/C797S, L858R, and/or L858R/C797S containing EGFR mutants useful for the therapeutic and/or prophylactic treatment of cancer, in particular T790M and C797S containing EGFR mutants.

The compound of formula (I) as described herein does have improved EGFR potency and selectivity for T790M/L858R, T790M/L858R/C797S, L858R and/or L858R/C797S containing EGFR mutants, in particular T790M and C797S containing EGFR mutants as well as improved physico-chemical properties.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert-butyl, sec-butyl, the isomeric pentyls, the isomeric hexyls, the isomeric heptyls and the isomeric octyls, particularly methyl, ethyl, propyl, butyl and pentyl. Particular examples of alkyl are methyl, ethyl, propyl, isopropyl, and tert-butyl. Methyl and ethyl are particular examples of “alkyl” in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of “cycloalkyl” are cyclopropyl, cyclobutyl, cyclopentyl and cyclohexyl, cycloheptyl and cyclooctanyl. Particular examples of “cycloalkyl” are cyclobutyl and cyclohexyl.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert-butoxy. Particular examples of “alkoxy” are methoxy and ethoxy. Ethoxy is a particular examples of “alkoxy” in the compound of formula (I).

The term “oxy”, alone or in combination, signifies the —O— group.

The terms “halogen” or “halo”, alone or in combination, denotes the substitution of said group with at least one halogen, particularly substituted with one to five halogens, particularly one to four halogens, i.e. one, two, three or four halogens. Examples of“halogen” or “halo” are fluorine, chlorine, bromine or iodine and particularly fluorine, chlorine or bromine. A particular “halogen” or “halo” is fluoro.

The term “haloalkyl”, alone or in combination, denotes an alkyl group substituted with at least one halogen, particularly substituted with one to five halogens, particularly one to three halogens. Particular examples of “haloalkyl” are fluoromethyl, fluoroethyl and fluoropropyl, more particular fluoropropyl.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH₂), the secondary amino group (—NH—), or the tertiary amino group (—N—).

The term “alkylamino”, alone or in combination, signifies an alkyl group linked to a —NH— group. The term “dialkylamino”, alone or in combination, signifies two alkyl groups linked to a —N-atom.

The term “sulfonyl”, alone or in combination, signifies the —SO₂— group.

The term “alkylsulfonyl”, alone or in combination, signifies an alkyl group linked to the —SO₂— group.

The term “cyano”, alone or in combination, signifies the —CN group.

The term “heterocycloalkyl” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Bicyclic means consisting of two cycles having one or two ring atoms in common. Examples of “heterocycloalkyl” are morpholinyl, piperidinyl, piperidyl, azetidinyl, piperazinyl, tetrahydro-1H-pyrrolo and hexahydropyrrolo[3,4-c]pyrrolyl. Particular examples of “heterocycloalkyl” are morpholinyl, piperidinyl, piperidyl and piperazinyl.

The terms “piperidinyl” and “piperidyl” are interchangeable and signify, alone or in combination, a saturated monocycle comprising 5 carbon ring atoms and one nitrogen ring atom.

The term “pharmaceutically acceptable salt” refers to those salts of the compound of formula (I) which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compound of formula (I) are the hydrochloride salts.

If one of the starting materials or a compound of formula (I) contains one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3^{rd} Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention the asymmetric carbon atom can be of the “R” or “S” configuration.

The invention thus relates to:

A compound according to the invention wherein A is —CH—;

A compound according to the invention wherein A is —N—;

A compound according to the invention wherein R¹ and R² are independently selected from fluoro and hydrogen;

A compound according to the invention wherein one of R¹ and R² is fluoro and the other one is hydrogen;

A compound according to the invention wherein R¹ and R² are both hydrogen at the same time;

A compound according to the invention wherein R³ is methyl;

A compound according to the invention wherein R⁴ is selected from morpholinyl, (alkyl)(halo)piperidinyl, alkylaminoalkyl, dialkylaminoalkyl, hydroxyalkylpiperidinylalkoxy, hydroxyalkylpiperidinylalkyl, hydroxypiperidinylalkyl, hydroxypiperidinylalkoxy, hydroxypiperidinyl, 1,4-diazabicyclo[3.2.1]octan-4-yl, hydroxyalkylpiperidinylalkylcycloalkyl, alkylsulfonylpiperidinyl, hydroxypiperidinylalkylcycloalkyl, morpholinylalkoxy, hydroxyalkylpiperidinylalkylazetidinyl, hydroxyalkylpiperazinyl, haloalkylpiperidinyl, (hydroxy)(alkyl)piperidinyl, alkyl(halohexahydropyrrolo[3,4-c]pyrrolyl), dialkylaminoalkylmorpholinyl, hydroxycycloalkyl(alkylamino)alkyl and haloalkyl(hexahydropyrrolo[3,4-c]pyrrolyl);

A compound according to the invention wherein R⁴ is selected from morpholinyl, (ethyl)(fluoro)piperidinyl, methylaminoethyl, dimethylaminoethyl, hydroxymethylpiperidinylethoxy, hydroxyethylpiperidinylethoxy, hydroxymethylpiperidinylmethyl, hydroxymethylpiperidinylethyl, hydroxyethylpiperidinylmethyl, hydroxyethylpiperidinylethyl, hydroxypiperidinylmethyl, hydroxypiperidinylethyl, hydroxypiperidinylethoxy, hydroxypiperidinyl, 1,4-diazabicyclo[3.2.1]octan-4-yl, hydroxymethylpiperidinylmethylcyclobutyl, methylsulfonylpiperidinyl, hydroxypiperidinylmethylcyclobutyl, morpholinylethoxy, hydroxymethylpiperidinylmethylazetidinyl, hydroxyethylpiperazinyl, fluoropropylpiperidinyl, (hydroxy)(methyl)piperidinyl, ethyl(fluorohexahydropyrrolo[3,4-c]pyrrolyl), dimethylaminomethylmorpholinyl, hydroxycyclohexyl(methylamino)methyl and fluoropropyl(hexahydropyrrolo[3,4-c]pyrrolyl);

A compound according to the invention wherein R⁴ is selected from heterocycloalkyl, dialkylaminoalkyl, hydroxyheterocycloalkyl alkyl, hydroxyheterocycloalkyl and hydroxyalkylheterocycloalkyl;

A compound according to the invention wherein R⁴ is selected from morpholinyl, dialkylaminoalkyl, hydroxypiperidinylalkyl, hydroxypiperidinyl and hydroxyalkylpiperazinyl; and

A compound according to the invention wherein R⁴ is selected from morpholinyl, dimethylaminoethyl, hydroxypiperidinylmethyl, hydroxypiperidinyl and hydroxyethylpiperazinyl.

The invention further relates to a compound selected from
2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(3 S,4S)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(3R,4R)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-[4-[2-(methylamino)ethyl]phenyl]indazol-2-yl]-2-[(6R)-6-fluoro-6,7-dihydro-5H-pyrrolo[1,2-c]imidazol-1-yl]-N-thiazol-2-yl-acetamide hydrochloride;
2-[4-(difluoromethyl)-6-[4-[2-(dimethylamino)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[4-(hydroxymethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[[4-(hydroxymethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(4-morpholinophenyl)indazol-2-yl]-2-(6,7-dihydro-5H-pyrrolo[1,2-c]imidazol-1-yl)-N-thiazol-2-yl-acetamide;
2-[4-(difluoromethyl)-6-[4-[2-[4-(hydroxymethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-(4-hydroxy-1-piperidyl)ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-(4-hydroxy-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[[4-(1-hydroxyethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[6-[4-(1,4-diazabicyclo[3.2.1]octan-4-yl)phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[4-(1-hydroxyethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[3-[[4-(hydroxymethyl)-1-piperidyl]methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-[4-(4-methylsulfonyl-1-piperidyl)phenyl]indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[3-[(4-hydroxy-1-piperidyl)methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-[4-(2-morpholinoethoxy)phenyl]indazol-2-yl]-2-[(6R)-6-fluoro-6,7-dihydro-5H-pyrrolo[1,2-c]imidazol-1-yl]-N-thiazol-2-yl-acetamide;
2-(4-(difluoromethyl)-6-(4-(3-((4-(hydroxymethyl)piperidin-1-yl)methyl)azetidin-1-yl)phenyl)-7-methyl-2H-indazol-2-yl)-2-((R)-6-fluoro-6,7-dihydro-5H-pyrrolo[1,2-c]imidazol-1-yl)-N-(thiazol-2-yl)acetamide;
2-[4-(difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[4-(1-hydroxyethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[1-(2-fluoropropyl)-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-(4-hydroxy-4-methyl-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[6-[4-[(3 aR,6aS)-2-ethyl-3a-fluoro-3,4,6,6a-tetrahydro-1H-pyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[(dimethylamino)methyl]morpholin-4-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-(4-hydroxy-1-piperidyl)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[[(4-hydroxycyclohexyl)-methyl-amino]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide; and
2-[6-[4-[(3aS,6aR)-2-(2-fluoropropyl)-1,3,3a,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide;
   or a pharmaceutically acceptable salt thereof.

The invention further relates to a compound selected from
2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-(dimethylamino)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-(4-hydroxy-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide; and
2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide
   or a pharmaceutically acceptable salt thereof.

A certain embodiment of the invention relates to a compound of formula (I) or a pharmaceutically acceptable salt thereof, wherein the compound of formula (I) is 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide.

A certain embodiment of the invention relates to a compound of formula (I) or a pharmaceutically acceptable salt thereof, wherein the compound of formula (I) is 2-[4-(difluoromethyl)-6-[4-[2-(dimethylamino)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide.

A certain embodiment of the invention relates to a compound of formula (I) or a pharmaceutically acceptable salt thereof, wherein the compound of formula (I) is 2-[4-(difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide.

A certain embodiment of the invention relates to a compound of formula (I) or a pharmaceutically acceptable salt thereof, wherein the compound of formula (I) is 2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide.

Processes for the manufacture of a compound of formula (I) as described herein are also an object of the invention.

The preparation of a compound of formula (I) of the present invention may be carried out in sequential or convergent synthetic routes. Syntheses of the compounds of the invention are shown in the following scheme 1 and in the description of specific examples. The skills required for carrying out the reactions and purifications of the resulting products are known to those skilled in the art. The substituents and indices used in the following description of the processes have the significance given herein before unless indicated to the contrary. The reaction sequence is not limited to the one displayed in scheme 1, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

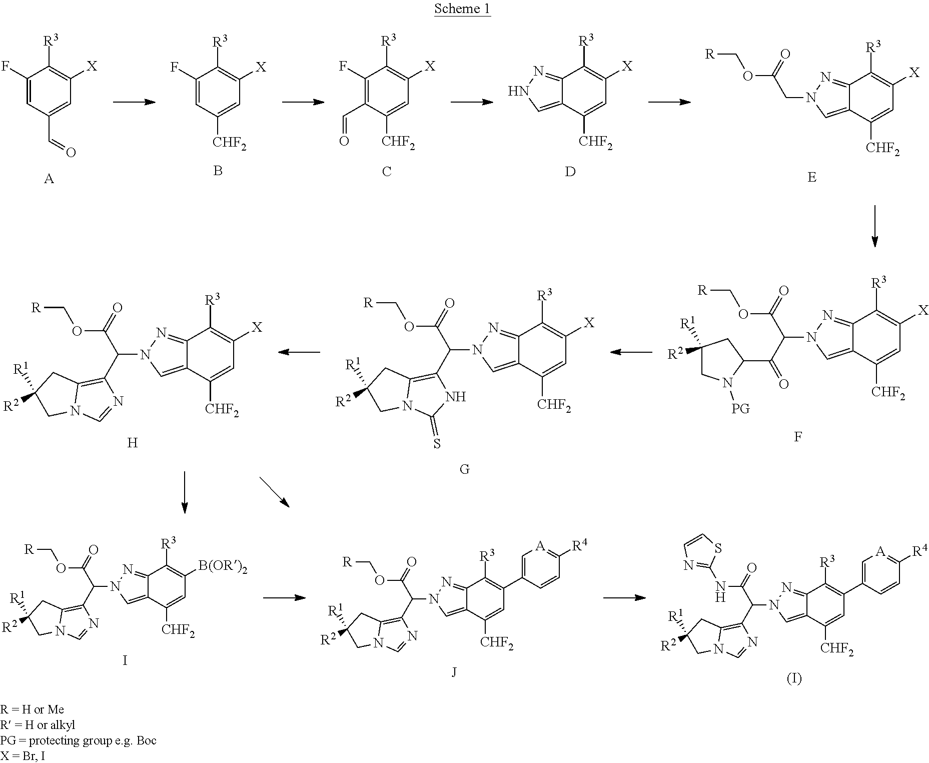

In scheme 1, A, R¹, R², R³ and R⁴ are as defined herein.

The compound of formula (I) may be prepared in accordance with Scheme 1 or as described in the examples. The starting materials are commercially available or may be prepared in accordance with known methods.

An aldehyde of formula A can be transformed into haloalkyl compound B by treatment with a deoxyfluorinating agent such as morpholinosulfur trifluoride in a solvent such as DCM at a temperature from 0° C. to room temperature. Lithiation with a base such as LDA in a solvent such as THE at a temperature such as −78° C. to −55° C. and quenching with an electrophilic formylating agent such as ethyl formate or DMF at a temperature between −78° C. and room temperature, gives aldehydes of formula C. These may be reacted directly with hydrazine hydrate in a solvent such as dioxane at elevated temperature to form indazoles D. Alternatively, following the procedure described by Lukin et al. (J. Org. Chem., 2006, 71, 8166), C may be first reacted with O-methylhydroxylamine hydrochloride in the presence of a base such as potassium carbonate in a solvent such as DME at an elevated temperature to afford an intermediate oxime, which is subsequently reacted with hydrazine hydrate at elevated temperatures to afford indazoles of formula D. An indazole of formula D can be alkylated with an alkylating agent such as ethyl bromoacetate or methyl bromoacetate in the presence or in the absence of a base such as triethylamine or cesium carbonate in a solvent such as dimethylacetamide or acetonitrile at ambient or elevated temperature to give an indazole of formula E. This compound can be deprotonated with a base such as LDA or LHMDS and treated with a proline derivative that was pre-activated by treatment with e.g CDI to give compounds of formula F. This reaction can be performed in a solvent such as THF at a temperature from −78° to room temperature. The protecting group of compound F can be cleaved by e.g. treatment with an acid such as HCl in dioxane or TFA. Subsequent treatment with potassium thiocyanate in a solvent such as EtOH at room temperature or slightly elevated temperature gives compounds of formula G. Conversion to imidazoles H can be achieved by treatment with hydrogen peroxide in a solvent such as acetic acid, or by treatment with hydrogen peroxide and para-toluenesulfonic acid in acetonitrile, or by treatment with Raney Nickel, or by other methods known in the art. Compounds of formula J can be obtained by using well-known methods such as Suzuki-, Sonogashira-, Negishi- and Buchwald-reactions as well as other well-known synthetic methods for functional group transformations. Alternatively bromide H may be converted to boronic acid or boronic ester I, for example by reaction with bis(pinacolato)diboron in the presence of a base such as KOAc and a catalyst such as Pd(dppf)Cl₂ in a solvent such as dioxane. Compounds H can subsequently be converted to compounds J by well-known methods such as Suzuki reactions. Conversion to compounds of formula (I) can be achieved by saponification with a base such as LiOH or NaOH in solvents such as EtOH, THF, MeOH and water, and subsequent amide coupling with aminothiazole or a derivative thereof with a coupling agent such as HATU. Alternatively, a direct ester-amide conversion can be achieved using aminothiazole and reagents such as trimethylaluminum or isopropylmagnesium chloride.

A corresponding pharmaceutically acceptable salt of the compound of formula (I) with an acid can be obtained by standard methods known to the person skilled in the art, e.g. by dissolving the compound of formula (I) in a suitable solvent such as e.g. dioxane or tetrahydrofuran and adding an appropriate amount of the corresponding acid. The products can usually be isolated by filtration or by chromatography. The conversion of a compound of formula (I) into a pharmaceutically acceptable salt with a base can be carried out by treatment of such a compound with such a base. One possible method to form such a salt is e.g. by addition of 1/n equivalents of a basic salt such as e.g. M(OH)n, wherein M=metal or ammonium cation and n=number of hydroxide anions, to a solution of the compound in a suitable solvent (e.g. ethanol, ethanol-water mixture, tetrahydrofuran-water mixture) and to remove the solvent by evaporation or lyophilisation. Particular pharmaceutically acceptable salts are hydrochloride, formate and trifluoroacetate.

Insofar as their preparation is not described in the examples, the compound of formula (I) as well as all intermediate products can be prepared according to analogous methods or according to the methods set forth herein.

It will be appreciated that the compounds of general formula (I) in this invention may be derivatised at functional groups to provide derivatives which are capable of conversion to the parent compound in vivo.

The invention thus also relates to a process for the preparation of a compound according to the invention, comprising the following steps:
(a) the reaction of a compound of formula (B1)

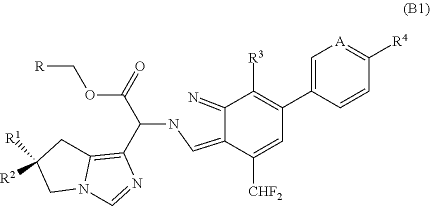

in a suitable solvent and in presence of a base, to arrive at a compound of formula (B2)

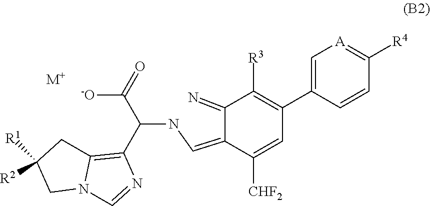

wherein M⁺ is Na⁺, Li⁺ or a protonated base;
(b) the reaction of the compound of formula (B2) in presence of an acid to yield a compound of formula (B3)

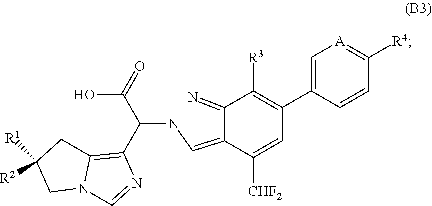

and
(c) the reaction of the compound of formula (B3) with a compound of formula (B4)

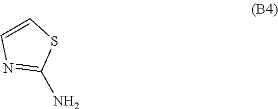

in the presence of a coupling agent and a base;
wherein A, R¹, R², R³ and R⁴ are as defined above, and wherein R is hydrogen or alkyl.

Conveniently R is hydrogen or methyl.

In step (a) the base can be for example LiOH or NaOH. Conveniently the base is LiOH.

In step (a) the concentration of the base can be between around 0.1M and between around 5M, particularly between around 0.2M and between around 4M, more particularly between around 0.5M and between around 2M. Conveniently the concentration of the base is around 1M.

Step (a) can be carried out in a solvent, like for example EtOH, THF, MeOH, water or a mixture thereof. Conveniently the solvent is MeOH, THF or a mixture thereof.

Convenient conditions for step (a) can be between around 0° C. to around 90° C., particularly between around 5° C. to around 80° C., more particularly between around 10° C. to around 50° C.

The reaction of step (b) can conveniently be carried out in a solvent. The solvent can be for example EtOH, MeOH, THF, water or a mixture thereof.

In step (b) the acid can be for example hydrochloric acid.

Convenient conditions for step (b) can be between around 0° C. to around 90° C., particularly between around 5° C. to around 80° C., more particularly between around 10° C. to around 50° C.

The reaction of step (c) can conveniently be carried out in a solvent. The solvent can be for example DMSO.

The reaction of step (c) can conveniently be carried out in presence of a base. The base can be for example DIPEA.

The reaction of step (c) can conveniently be carried out in presence of a coupling agent. The coupling agent can be for example HATU.

Convenient conditions for the reaction of step (c) can be between around 0° C. to around 90° C., particularly between around 5° C. to around 80° C., more particularly between around 10° C. to around 50° C.

The invention also relates to a compound according to the invention when manufactured according to a process of the invention.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compound of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compound of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention thus also relates in particular to:

A compound according to the invention for use as therapeutically active substance;

A pharmaceutical composition comprising a compound according to the invention and a therapeutically inert carrier;

A compound according to the invention for use in the treatment or prophylaxis of cancer;

A compound according to the invention for use in the treatment or prophylaxis of non-small cell lung cancer;

The use of a compound according to the invention for the treatment or prophylaxis of cancer, in particular non-small cell lung cancer;

The use of a compound according to the invention for the preparation of a medicament for the treatment or prophylaxis of cancer, in particular non-small cell lung cancer; and

A method for the treatment or prophylaxis of cancer, in particular non-small cell lung cancer, which method comprises administering an effective amount of a compound according to the invention to a patient in need thereof.

A certain embodiment of the invention relates to a pharmaceutical composition comprising the compound of formula (I) as described herein, or a pharmaceutically acceptable salt thereof, and a pharmaceutically acceptable auxiliary substance.

A certain embodiment of the invention relates to the compound of formula (I) as described herein, or a pharmaceutically acceptable salt thereof, for the use in the treatment or prophylaxis of cancer, in particular non-small cell lung cancer, characterized by at least one EGFR mutation selected from T790M/L858R, T790M/L858R/C797S, L858R and L858R/C797S.

A certain embodiment of the invention relates to a method for the treatment or prophylaxis of cancer, in particular non-small cell lung cancer, wherein at least one EGFR mutation selected from T790M/L858R, T790M/L858R/C797S, L858R and L858R/C797S is present in the cancer, which method comprises administering an effective amount of a compound of formula (I) as described herein, or a pharmaceutically acceptable salt thereof, to a patient in need thereof.

Furthermore, the invention includes all substituents in its corresponding deuterated form, wherever applicable, of the compound of formula (I).

Furthermore, the invention includes all optical isomers, i.e. diastereoisomers, diastereomeric mixtures, racemic mixtures, all their corresponding enantiomers and/or tautomers as well as their solvates, wherever applicable, of the compound of formula (I).

The compound of formula (I) may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains>90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

Also an embodiment of the present invention is a compound of formula (I) as described herein, when manufactured according to any one of the described processes.

The compound of formula (I) or a pharmaceutically acceptable salt thereof can be used as a medicament (e.g. in the form of a pharmaceutical preparation). The pharmaceutical preparation can be administered internally, such as orally (e.g. in the form of tablets, coated tablets, dragees, hard and soft gelatin capsules, solutions, emulsions or suspensions), nasally (e.g. in the form of nasal sprays), rectally (e.g. in the form of suppositories) or topical ocularly (e.g. in the form of solutions, ointments, gels or water soluble polymeric inserts). However, the administration can also be effected parenterally, such as intramuscularly, intravenously, or intraocularly (e.g. in the form of sterile injection solutions).

The compound of formula (I) or a pharmaceutically acceptable salt thereof can be processed with pharmaceutically inert, inorganic or organic adjuvants for the production of tablets, coated tablets, dragees, hard gelatin capsules, injection solutions or topical formulations Lactose, corn starch or derivatives thereof, talc, stearic acid or its salts etc. can be used, for example, as such adjuvants for tablets, dragees and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparation can contain preservatives, solubilizers, viscosity-increasing substances, stabilizers, wetting agents, emulsifiers, sweeteners, colorants, flavorants, salts for varying the osmotic pressure, buffers, masking agents or antioxidants. The pharmaceutical preparation can also contain still other therapeutically valuable substances.

The dosage can vary in wide limits and will, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

### Pharmaceutical Compositions

The compound of formula (I) or a pharmaceutically acceptable salt thereof can be used as a therapeutically active substance, e.g. in the form of a pharmaceutical preparation. The pharmaceutical preparation can be administered orally, e.g. in the form of tablets, coated tablets, dragees, hard and soft gelatin capsules, solutions, emulsions or suspensions. The administration can, however, also be effected rectally, e.g. in the form of suppositories, or parenterally, e.g. in the form of injection solutions.

The compound of formula (I) or a pharmaceutically acceptable salt thereof can be processed with pharmaceutically inert, inorganic or organic carriers for the production of a pharmaceutical preparation. Lactose, corn starch or derivatives thereof, talc, stearic acids or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi liquid or liquid polyols and the like.

The pharmaceutical preparation can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula (I) or a pharmaceutically acceptable salt thereof and a therapeutically inert carrier are also provided by the present invention, as is a process for their production, which comprises bringing a compound of formula (I) and/or pharmaceutically acceptable salts thereof and, if desired, one or more other therapeutically valuable substances into a galenical administration form together with one or more therapeutically inert carriers.

The dosage can vary within wide limits and will, have to be adjusted to the individual requirements in each particular case. In the case of oral administration the dosage for adults can vary from about 0.01 mg to about 1000 mg per day of a compound of general formula (I) or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparation conveniently contains about 1-500 mg, particularly 1-100 mg, of a compound of formula (I). Examples of compositions according to the invention are:

### EXAMPLE A

Tablets of the following composition are manufactured in the usual manner:

TABLE 1
possible tablet composition

mg/tablet

| ingredient | 5 | 25 | 100 | 500 |
|---|---|---|---|---|
| (1) Compound of formula (I) | 5 | 25 | 100 | 500 |
| (2) Lactose Anhydrous DTG | 125 | 105 | 30 | 150 |
| (3) Sta-Rx 1500 | 6 | 6 | 6 | 60 |
| (4) Microcrystalline Cellulose | 30 | 30 | 30 | 450 |
| (5) Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### EXAMPLE B-1

Capsules of the following composition are manufactured:

TABLE 2
possible capsule ingredient composition

mg/capsule

| ingredient | 5 | 25 | 100 | 500 |
|---|---|---|---|---|
| (1) Compound of formula (I) | 5 | 25 | 100 | 500 |
| (2) Hydrous Lactose | 159 | 123 | 148 | — |
| (3) Corn Starch | 25 | 35 | 40 | 70 |
| (4) Talk | 10 | 15 | 10 | 25 |
| (5) Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula (I), lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### EXAMPLE B-2

Soft Gelatin Capsules of the following composition are manufactured:

TABLE 3
possible soft gelatin capsule ingredient composition

| ingredient | mg/capsule |
|---|---|
| Compound of formula (I) | 5 |
| Yellow wax | 8 |
| Hydrogenated Soya bean oil | 8 |
| Partially hydrogenated plant oils | 34 |
| Soya bean oil | 110 |
| Total | 165 |

TABLE 4
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula (I) is dissolved in a warm melting of the other ingredients and the mixture is filled into soft gelatin capsules of appropriate size. The filled soft gelatin capsules are treated according to the usual procedures.

### EXAMPLE C

Suppositories of the following composition are manufactured:

TABLE 5
possible suppository composition

| ingredient | mg/supp. |
|---|---|
| Compound of formula (I) | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula (I) is added thereto and stirred until it has dispersed completely. The mixture is poured into suppository moulds of suitable size, left to cool; the suppositories are then removed from the moulds and packed individually in wax paper or metal foil.

### EXAMPLE D

Injection solutions of the following composition are manufactured:

TABLE 6
possible injection solution composition

| ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

### Manufacturing Procedure

The compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### EXAMPLE E

Sachets of the following composition are manufactured:

TABLE 7
possible sachet composition

| ingredient | mg/sachet |
|---|---|
| Compound of formula (I) | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula (I) is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Examples

### Abbreviations

2-MeTHF=2-methyltetrahydrofuran; AcOH=acetic acid; ATP=adenosine triphosphate; BOC=tert-butyloxycarbonyl; DCM=dichloromethane; CAS=chemical abstract service; CDI=1,1′-carbonyldiimidazole; dba=dibenzylideneacetone; DCM=dichloromethane; DIPEA=diisopropylethylamine; DME=dimethoxyethane; DMF=dimethylformamide; DMSO=diemethyl sulfoxide; dppf=1,1′-Bis(diphenylphosphino)ferrocene; ESI=electrospray ionization; EtOAc=ethyl acetate; EtOH=ethanol; HATU=hexafluorophosphate azabenzotriazole tetramethyl uronium; HPLC=high performance liquid chromatography; LDA=lithium diisopropylamide; LHMDS=Lithium bis(trimethylsilyl)amide; MeOH=methanol; MS=mass spectrometry; NMR=nuclear magnetic resonance; RP-HPLC; reversed-phase-HPLC; rt=room temperature; SFC=supercritical fluid chromatography; TBAF=tetra-n-butylammonium fluoride; TBDMS-Cl=tert-butyldimethylsilyl chloride; TBME=tert-butylmethylether; THE=tetrahydrofuran.

The following examples are provided for illustration of the invention. They should not be considered as limiting the scope of the invention, but merely as being representative thereof.

### Intermediate 1

### Ethyl 2-[6-bromo-4-(difluoromethyl)-7-methyl-indazol-2-yl]-2-[(6R)-6-fluoro-6,7-dihydro-5H-pyrrolo[1,2-c]imidazol-1-yl]acetate

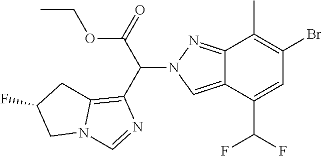 [SMILES: CCOC(=O)C(c1ncn2c1C[C@@H](F)C2)n1cc2c(C(F)F)cc(Br)c(C)c2n1]

### Step 1: 1-bromo-5-(difluoromethyl)-3-fluoro-2-methylbenzene

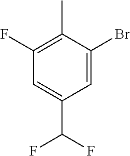

A solution of 3-bromo-5-fluoro-4-methylbenzaldehyde (CAS 1370411-47-4, 20.5 g, 89.7 mmol, Eq: 1.0) in DCM (98 mL) was cooled with ice bath. Morpholinosulfur trifluoride (CAS 51010-74 3, 24.8 g, 17.3 mL, 135 mmol, Eq: 1.5) was added in portions. The reaction mixture was stirred at 0-5° C. for 20 min, then stirred for 16 h at rt. With ice cooling, sat. aq. NaHCO₃(300 mL) was added carefully. The reaction mixture was stirred for 1 h at rt. The reaction mixture was poured into DCM and washed with water. The organic layer was dried over Na₂SO₄ and concentrated in vacuo. The crude material was purified by flash chromatography (silica gel, 120 g, 100% pentane) to give the title compound as a colourless oil (18.6 g, 87% yield). ¹H NMR (300 MHz, chloroform-d) δ=7.50 (s, 1H), 7.16 (d, J=9.1 Hz, 1H), 6.57 (t, J=56.0 Hz, 1H), 2.50-2.22 (m, 3H)

### Step 2: 6-bromo-4-(difluoromethyl)-7-methyl-1H-indazole

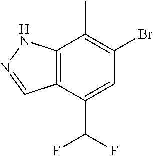

A solution of 1-bromo-5-(difluoromethyl)-3-fluoro-2-methylbenzene (26.4 g, 110 mmol, Eq: 1.0) in THE (240 mL) was cooled to −75° C. LDA (2 M in THF/heptane/ethylbenzene, 66.3 mL, 133 mmol, Eq: 1.2) was added dropwise below −70° C. The reaction mixture was stirred for 30 min at −75° C. Ethyl formate (16.4 g, 17.7 mL, 220 mmol, Eq: 2.0) was added below −70° C. The reaction mixture was stirred at −75° C. for 30 min. AcOH (16.6 g, 15.8 mL, 277 mmol, Eq: 2.5) was added below −55° C. The reaction mixture was allowed to warm up to rt and poured into EtOAc and washed with dilute aq. HCl, water and brine. The organic layer was dried over Na₂SO₄ and concentrated in vacuo to give presumed 4-bromo-6-(difluoromethyl)-2-fluoro-3-methylbenzaldehyde as a yellow oil (29.5 g) which was used without further purification. The crude presumed 4-bromo-6-(difluoromethyl)-2-fluoro-3-methylbenzaldehyde (29.5 g) was dissolved in DME (150 mL). O-Methylhydroxylamine hydrochloride (10.2 g, 122 mmol, Eq: 1.84) and K₂CO₃ (30.6 g, 221 mmol, Eq: 3.34) were added. The reaction mixture was stirred for 2.5 h at 45° C. then filtered through sintered glass and washed with DME (2×). The filtrate was concentrated in vacuo. The oxime ether intermediate was dissolved in DMSO (150 mL). Hydrazine hydrate (83 g, 80.5 mL, 1.66 mol, Eq: 25) was added. The reaction mixture was stirred for 3 h at 110° C. The reaction mixture was poured into EtOAc/THF 5:1 and washed with water and brine. The organic layer was dried over Na₂SO₄ and concentrated in vacuo. The crude material was purified by flash chromatography (silica gel, 2×120 g, 0% to 30% EtOAc in heptane) to give the title compound as a white solid (13.5 g, 74% yield). m/z 258.9, 260.8 [M+H]⁺, ESI pos, Br isotopes.

### Step 3: Ethyl 2-[6-bromo-4-(difluoromethyl)-7-methyl-indazol-2-yl]acetate

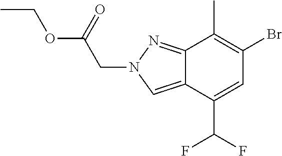 [SMILES: CCOC(=O)Cn1cc2c(C(F)F)cc(Br)c(C)c2n1]

To a solution of 6-bromo-4-(difluoromethyl)-7-methyl-1H-indazole (19 g, 72.8 mmol, Eq: 1.0) in DMF (75 mL) was added ethyl 2-bromoacetate (18.2 g, 12.2 mL, 109 mmol, Eq: 1.5). The reaction mixture was stirred for 16 h at 100° C. The reaction mixture was poured into EtOAc and washed with water and brine. The organic layer was dried over Na₂SO₄ and concentrated in vacuo. The crude material was purified by flash chromatography (silica gel, 2×120 g, 0% to 20% EtOAc in heptane) to give the title compound as a yellow solid (21.2 g, 80% yield). m/z 346.9, 348.8, [M+H]⁺, ESI pos, Br isotopes.

### Step 4: Tert-Butyl (25,4R)-2-[2-[6-bromo-4-(difluoromethyl)-7-methyl-indazol-2-yl]-3-ethoxy-3-oxo-propanoyl]-4-fluoro-pyrrolidine-1-carboxylate

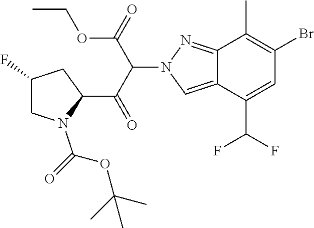 [SMILES: CCOC(=O)C(C(=O)[C@@H]1C[C@@H](F)CN1C(=O)OC(C)(C)C)n1cc2c(C(F)F)cc(Br)c(C)c2n1]

### Preparation of tert-butyl (2S,4R)-4-fluoro-2-(imidazole-1-carbonyl)pyrrolidine-1-carboxylate

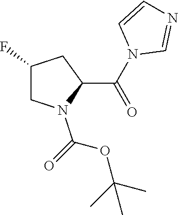 [SMILES: CC(C)(C)OC(=O)N1C[C@H](F)C[C@H]1C(=O)n1ccnc1]

To a solution of (2S,4R)-1-tert-butoxycarbonyl-4-fluoro-pyrrolidine-2-carboxylic acid (CAS 203866-14-2, 30 g, 129 mmol, Eq: 1.0) in DCM (300 mL) was added 1,1′-carbonyldiimidazole (25 g, 154 mmol, Eq: 1.2) in portions at 0° C. The reaction mixture was stirred for 3 h at rt. The reaction mixture was washed with water (3×) and 1M aq. NaHCO₃(lx). The organic layer was dried over Na₂SO₄ and concentrated in vacuo at 30° C. to give tert-butyl (2S,4R)-4-fluoro-2-(imidazole-1-carbonyl)pyrrolidine-1-carboxylate (36.6 g, 129 mmol, 100% yield) as a white solid which was stored at −20° C. prior to use. ¹H NMR (chloroform-d, 300 MHz) δ 8.27 (s, 1H), 7.56 (br d, 1H, J=1.4 Hz), 7.15 (br d, 1H, J=12.1 Hz), 4.9-5.2 (m, 1H), 3.6-4.1 (m, 2H), 2.0-2.9 (m, 2H), 1.2-1.5 (m, 9H).

KO^{t}Bu (4.53 g, 40.3 mmol, Eq: 2.0) was dissolved in THF (18 mL). The reaction mixture was cooled to −55° C. A solution of ethyl 2-[6-bromo-4-(difluoromethyl)-7-methyl-indazol-2-yl]acetate (7 g, 20.2 mmol, Eq: 1) in THF (24 mL) was added dropwise below −50° C. The reaction mixture was stirred for 1 h between −50° C. and −55° C. A solution of previously-prepared tert-butyl (2S,4R)-4-fluoro-2-(imidazole-1-carbonyl)pyrrolidine-1-carboxylate (6.85 g, 24.2 mmol, Eq: 1.2) in THF (50 mL) was added dropwise below −50° C. The reaction mixture was stirred for 15 min at −50° C., then allowed to warm up to −30° C. 10% aqueous citric acid (60 mL) was added below −20° C., and the mixture was stirred for 1 h at 0° C. The reaction mixture was poured into EtOAc and washed with water and brine. The organic layer was dried over Na₂SO₄ and concentrated in vacuo at 30° C. to give the title compound as a yellow amorphous semisolid (12.9 g, 20.2 mmol, 88% purity, 100% yield). m/z 562.1, 563.9 [M+H]⁺, ESI pos, Br isotopes.

### Step 5: Ethyl 2-[6-bromo-4-(difluoromethyl)-7-methyl-indazol-2-yl]-2-[(6R)-6-fluoro-3-thioxo-2,5,6,7-tetrahydropyrrolo[1,2-c]imidazol-1-yl]acetate

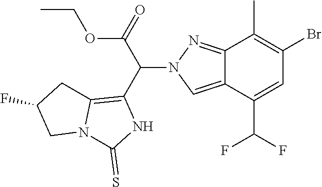 [SMILES: CCOC(=O)C(c1[nH]c(=S)n2c1C[C@@H](F)C2)n1cc2c(C(F)F)cc(Br)c(C)c2n1]

To a solution of tert-butyl (2 S,4R)-2-[2-[6-bromo-4-(difluoromethyl)-7-methyl-indazol-2-yl]-3-ethoxy-3-oxo-propanoyl]-4-fluoro-pyrrolidine-1-carboxylate (12.9 g, 20.2 mmol, Eq: 1) in ethanol (24 mL) was added HCl (1.25 M in ethanol, 80.6 mL, 101 mmol, Eq: 5). The reaction mixture was stirred for 1 h at 55° C. The reaction mixture was cooled to rt, then water (6 mL) and potassium thiocyanate (2.55 g, 26.2 mmol, Eq: 1.3) were added. The reaction mixture was stirred for 30 min at rt. The ethanol was removed in vacuo at 30° C., and pyridine (23.9 g, 24.5 mL, 302 mmol, Eq: 15) was added. The reaction mixture was stirred at rt for 75 min. The reaction mixture was poured into EtOAc and washed with 2 N aq. HCl (until the aqueous phase was pH 1), water and brine. The organic layer was dried over Na₂SO₄ and concentrated in vacuo to give the title compound as a yellow semisolid (9.65 g, 60% purity, 57% yield). m/z 502.9, 505.9 [M+H]⁺, ESI pos, Br isotopes.

### Step 6: Ethyl 2-[6-bromo-4-(difluoromethyl)-7-methyl-indazol-2-yl]-2-[(6R)-6-fluoro-6,7-dihydro-5H-pyrrolo[1,2-c]imidazol-1-yl]acetate

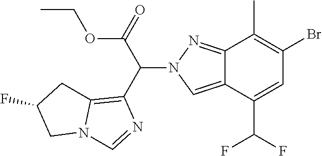 [SMILES: CCOC(=O)C(c1ncn2c1C[C@@H](F)C2)n1cc2c(C(F)F)cc(Br)c(C)c2n1]

To a suspension of p-toluenesulfonic acid monohydrate (10.9 g, 57.5 mmol, Eq: 5) in acetonitrile (70 mL) was added hydrogen peroxide (35% aq., 8.38 g, 7.42 mL, 86.3 mmol, Eq: 7.5) dropwise at 0-3° C. to give a colorless solution. After 10 min, ethyl 2-[6-bromo-4-(difluoromethyl)-7-methyl-indazol-2-yl]-2-[(6R)-6-fluoro-3-thioxo-2,5,6,7-tetrahydropyrrolo[1,2-c]imidazol-1-yl]acetate (9.65 g, 11.5 mmol, Eq: 1) in acetonitrile (28 mL) was added dropwise below 8° C. The reaction mixture was stirred for 1.5 h at 0° C. The reaction mixture was poured into EtOAc and washed with Na₂CO₃ solution and brine. The organic layer was dried over Na₂SO₄ and concentrated in vacuo. The crude material was purified by flash chromatography (silica gel, 120 g, 0% to 60% (EtOAc/EtOH/aq. NH₃ 75:25:2) in heptane) to give the title compound as a yellow foam (3.96 g, 73% yield). m/z 469.1, 471.1 [M+H]⁺, ESI pos.

### Intermediate 2

### Ethyl 2-[6-bromo-4-(difluoromethyl)-7-methyl-indazol-2-yl]-2-(6,7-dihydro-5H-pyrrolo[1,2-c]imidazol-1-yl)acetate

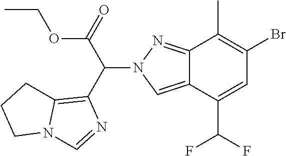 [SMILES: CCOC(=O)C(c1ncn2c1CCC2)n1cc2c(C(F)F)cc(Br)c(C)c2n1]

### Step 1: Tert-Butyl (2R)-2-[2-[6-bromo-4-(difluoromethyl)-7-methylindazol-2-yl]-3-ethoxy-3-oxopropanoyl]pyrrolidine-1-carboxylate

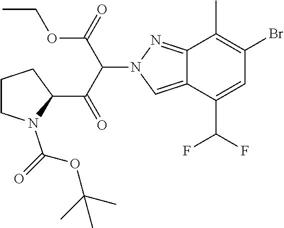 [SMILES: CCOC(=O)C(C(=O)[C@@H]1CCCN1C(=O)OC(C)(C)C)n1cc2c(C(F)F)cc(Br)c(C)c2n1]

Ethyl 2-[6-bromo-4-(difluoromethyl)-7-methyl-indazol-2-yl]acetate, prepared as described in Intermediate 1, Step 3 (0.65 g, 1.87 mmol, Eq: 1) was dissolved in THF (7.58 mL) and cooled to −75° C. LDA (2 M in THF, 1.12 mL, 2.25 mmol, Eq: 1.20) was added dropwise within 5 min. The reaction mixture was stirred for 40 min at −75° C. A solution of tert-butyl (2S)-2-(imidazole-1-carbonyl)pyrrolidine-1-carboxylate (prepared from (2S)-1-tert-butoxycarbonyl-4-fluoro-pyrrolidine-2-carboxylic acid by analogy with Intermediate 1, Step 4) (0.77 g, 2.9 mmol, Eq: 1.55) in THE (7.58 mL) was added slowly at −75° C., stirred for 30 min at −75° C. then allowed to warm up to rt and stirred for 18 h at rt. After the addition of sat. aq. NH₄Cl, the reaction mixture was extracted twice with EtOAc. The organic layers were washed with water. The combined organic layers were dried over Na₂SO₄ and concentrated in vacuo to give the title compound (1.41 g, 72% purity, 99%, yield) which was used in the next step without further purification. m/z 544.1, 546.0 [M+H]⁺, ESI pos. Br isotopes.

### Step 2: Ethyl 2-[6-bromo-4-(difluoromethyl)-7-methyl-indazol-2-yl]-2-(3-thioxo-2,5,6,7-tetrahydropyrrolo[1,2-c]imidazol-1-yl)acetate

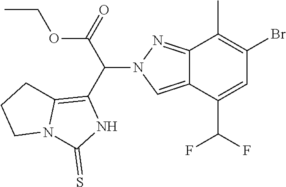 [SMILES: CCOC(=O)C(c1[nH]c(=S)n2c1CCC2)n1cc2c(C(F)F)cc(Br)c(C)c2n1]

By analogy with Intermediate 1, Step 5, tert-butyl (2R)-2-[2-[6-bromo-4-(difluoromethyl)-7-methylindazol-2-yl]-3-ethoxy-3-oxopropanoyl]pyrrolidine-1-carboxylate (1.4 g, 72% purity, 1.85 mmol) was treated with HCl 4 M in dioxane and potassium thiocyanate to give the title compound as a brown oil (1.07 g, 85% purity, 100% yield) which was used in the next step without further purification. m/z 485.0, 486.9 [M+H]⁺, ESI pos, Br isotopes.

### Step 3: ethyl 2-[6-bromo-4-(difluoromethyl)-7-methyl-indazol-2-yl]-2-(6,7-dihydro-5H-pyrrolo[1,2-c]imidazol-1-yl)acetate

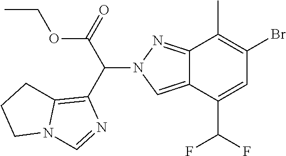 [SMILES: CCOC(=O)C(c1ncn2c1CCC2)n1cc2c(C(F)F)cc(Br)c(C)c2n1]

By analogy with Intermediate 1, Step 6, ethyl 2-[6-bromo-4-(difluoromethyl)-7-methyl-indazol-2-yl]-2-(3-thioxo-2,5,6,7-tetrahydropyrrolo[1,2-c]imidazol-1-yl)acetate (1.06 g, 85% purity, 1.86 mmol) was treated with hydrogen peroxide and p-toluenesulfonic acid monohydrate to give the title compound as a yellow foam (360 mg, 43% yield). m/z 453.0, 454.9 [M+H]⁺, ESI pos, Br isotopes.

### Intermediate 3

### Ethyl 2-[4-(difluoromethyl)-7-methyl-6-(4,4,5,5-tetramethyl-1,3,2-dioxaborolan-2-yl)indazol-2-yl]-2-[(6R)-6-fluoro-6,7-dihydro-5H-pyrrolo[1,2-c]imidazol-1-yl]acetate

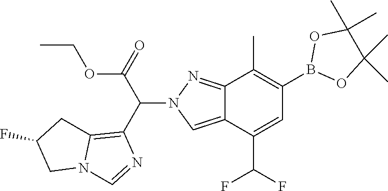 [SMILES: CCOC(=O)C(c1ncn2c1C[C@@H](F)C2)n1cc2c(C(F)F)cc(B3OC(C)(C)C(C)(C)O3)c(C)c2n1]

To a solution of Intermediate 1 (200 mg, 424 μmol, Eq: 1.0) in dioxane (2.02 mL) were added bis(pinacolato)diboron (162 mg, 637 μmol, Eq: 1.5), KOAc (208 mg, 2.12 mmol, Eq: 5.0) and Pd(dppf)Cl₂·CH₂Cl₂ (62.1 mg, 84.9 μmol, Eq: 0.20) under nitrogen at rt. The mixture was stirred for 6 h at 90° C. Water and EtOAc were added to the reaction mixture and the organic layer was separated, dried over Na₂SO₄ and evaporated. The crude material was purified by flash chromatography (silica gel, 25 g, 0% to 10% MeOH in EtOAc) to give the title compound as a light brown solid (80 mg, 29% yield). m/z 219.2 [M+H]⁺, ESI pos.

### Example 1

### 2-[4-(Difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide

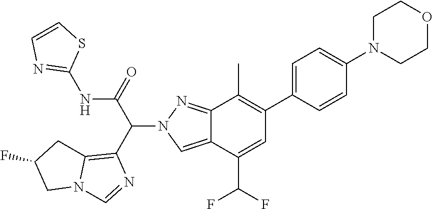 [SMILES: Cc1c(-c2ccc(N3CCOCC3)cc2)cc(C(F)F)c2cn(C(C(=O)Nc3nccs3)c3ncn4c3C[C@@H](F)C4)nc12]

### Step 1: Ethyl 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]acetate

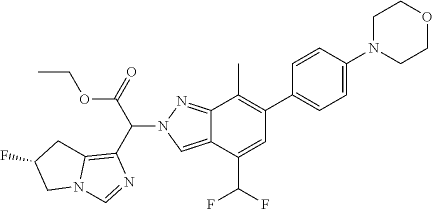 [SMILES: CCOC(=O)C(c1ncn2c1C[C@@H](F)C2)n1cc2c(C(F)F)cc(-c3ccc(N4CCOCC4)cc3)c(C)c2n1]

A suspension of Intermediate 1 (0.5 g, 1.06 mmol, Eq: 1.0), (4-morpholinophenyl)boronic acid (CAS 186498-02-2, 329 mg, 1.59 mmol, Eq: 1.5) and K₂CO₃ (183 mg, 1.33 mmol, Eq: 1.25) in 2-MeTHF (6 mL), water (1 mL) was degassed with argon for 10 min. Dichloro[bis(diphenylphosphinophenyl)ether]palladium(II) (CAS 205319-06-8, 91 mg, 127 μmol, Eq: 0.12) was added. The reaction mixture was stirred for 5 h at 85° C. AcOH (191 mg, 182 μL, 3.18 mmol, Eq: 3.0) was added. The reaction mixture was poured into EtOAc/THF 2:1 and washed with water and brine. The organic layer was dried over Na₂SO₄ and concentrated in vacuo. The crude material was purified by flash chromatography (silica gel, 12 g, 0% to 50% (EtOAc/EtOH/aq. NH₃ 75:25:2) in heptane) to give the title compound as an off-white solid (433 mg, 743 μmol, 70% yield). m/z 554.4 [M+H]⁺, ESI pos.

### Step 2: 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide

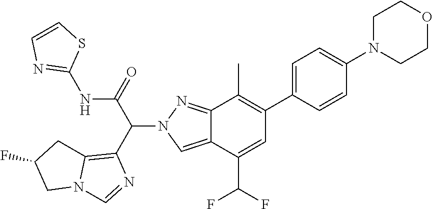 [SMILES: Cc1c(-c2ccc(N3CCOCC3)cc2)cc(C(F)F)c2cn(C(C(=O)Nc3nccs3)c3ncn4c3C[C@@H](F)C4)nc12]

To a solution of ethyl 2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]acetate (720 mg, 1.3 mmol, Eq: 1.0) in THE (9 mL) and MeOH (9 mL) was added LiOH (1 M aq., 2.6 mL, 2.6 mmol, Eq: 2.0). The reaction mixture was stirred for 2 h at rt. HCl (5 N aq., 520 μL, 2.6 mmol, Eq: 2.0) was added (pH 6). Toluene was added, the reaction mixture was concentrated in vacuo. The carboxylic acid was dissolved in DMSO (6 mL), thiazol-2-amine (195 mg, 1.95 mmol, Eq: 1.5), DIPEA (840 mg, 1.14 mL, 6.5 mmol, Eq: 5.0) and HATU (742 mg, 1.95 mmol, Eq: 1.5) were added. The reaction mixture was stirred for 1.5 hat rt. The reaction mixture was poured into EtOAc/THF 2:1 and washed with water and brine. The organic layer was dried over Na₂SO₄ and concentrated in vacuo. The crude material was purified by flash chromatography (silica gel, 40 g, 0% to 5% MeOH in DCM). The product was freeze-dried to give the title compound as an off-white solid (501 mg, 63% yield). m/z 608.3 [M+H]⁺, ESI pos.

### Example 2

### 2-[4-(Difluoromethyl)-6-[4-[(3S,4S)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide or 2-[4-(Difluoromethyl)-6-[4-[(3R,4R)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

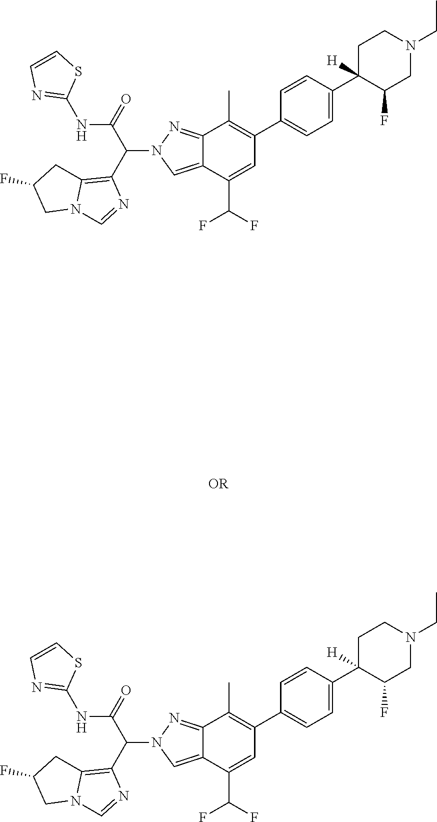 [SMILES: CCN1CC[C@@H](c2ccc(-c3cc(C(F)F)c4cn(C(C(=O)Nc5nccs5)c5ncn6c5C[C@@H](F)C6)nc4c3C)cc2)[C@H](F)C1 ; CCN1CC[C@H](c2ccc(-c3cc(C(F)F)c4cn(C(C(=O)Nc5nccs5)c5ncn6c5C[C@@H](F)C6)nc4c3C)cc2)[C@@H](F)C1]

### Step 1: Tert-Butyl (3S,4S)-4-(4-bromophenyl)-3-hydroxy-piperidine-1-carboxylate and tert-butyl (3R,4R)-4-(4-bromophenyl)-3-hydroxy-piperidine-1-carboxylate

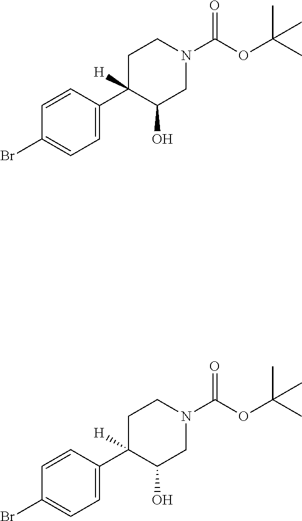 [SMILES: CC(C)(C)OC(=O)N1CC[C@@H](c2ccc(Br)cc2)[C@H](O)C1 ; CC(C)(C)OC(=O)N1CC[C@H](c2ccc(Br)cc2)[C@@H](O)C1]

A solution of tert-butyl 4-(4-bromophenyl)-3,6-dihydro-2H-pyridine-1-carboxylate (CAS 273727 44-9, 30 g, 79.8 mmol, Eq: 1.0, 90% purity) in THF (275 mL) was cooled to 0° C. Borane tetrahydrofuran complex (1.0 M solution in THF, CAS 14044-65-6, 87.8 mL, 87.8 mmol, Eq: 1.1) was added dropwise at 0° C. After the addition was complete, the ice bath was removed and the reaction mixture was stirred for 16 h at rt. The reaction mixture was cooled to 0° C. NaOH (5 M in water, 40 mL, 200 mmol, Eq: 2.51) was added dropwise and the reaction mixture was stirred for 30 min at 0° C. Hydrogen peroxide (35 wt. % solution in water, 19.4 g, 17.5 mL, 200 mmol, Eq: 2.5) was added and the reaction mixture was stirred for 2.5 h at 50° C. The reaction mixture was cooled to rt and the excess of peroxide was quenched by addition of 2 M aq. Na₂S₂O₃. The mixture was diluted with ethyl acetate and water. The aqueous layer was back-extracted twice with ethyl acetate. The combined organic layers were dried over Na₂SO₄ and concentrated in vacuo. The crude product was suspended in diisopropyl ether and filtered. The solids were washed with diisopropyl ether and dried in vacuo to give the title compound as a white solid (26.74 g, 92% yield). m/z 258.1 [M-BOC+H]⁺, ESI pos.

### Step 2: Tert-Butyl (3S,4S)-4-(4-bromophenyl)-3-fluoro-piperidine-1-carboxylate and tert-butyl (3R,4R)-4-(4-bromophenyl)-3-fluoro-piperidine-1-carboxylate

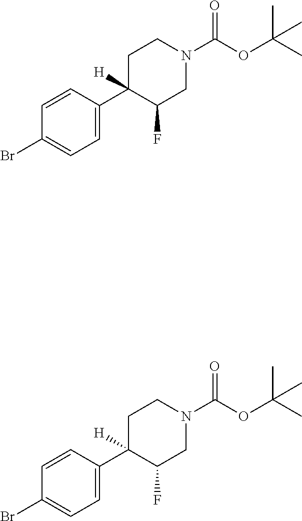 [SMILES: CC(C)(C)OC(=O)N1CC[C@@H](c2ccc(Br)cc2)[C@H](F)C1 ; CC(C)(C)OC(=O)N1CC[C@H](c2ccc(Br)cc2)[C@@H](F)C1]

A solution of tert-butyl (3S,4S)-4-(4-bromophenyl)-3-hydroxy-piperidine-1-carboxylate and tert-butyl (3R,4R)-4-(4-bromophenyl)-3-hydroxy-piperidine-1-carboxylate (37.2 g, 99.2 mmol, Eq: 1.0) in dichlormethane (500 mL) was cooled to −78° C. Deoxofluor® (50% solution in THF, CAS 202289-38-1, 79 g, 65.8 mL, 179 mmol, Eq: 1.8) was added dropwise at −78° C. The reaction mixture was allowed to slowly warm to rt and stirred for 16 h at rt. The reaction mixture was quenched with sat. aq. NaHCO₃. The mixture was stirred for 30 min (pH 7). The organic layer was separated. The aqueous layer was back-extracted with DCM. The combined organic layers were dried over Na₂SO₄ and concentrated in vacuo. The crude material was adsorbed on Isolute HM-N and purified by flash chromatography (silica gel, 330 g, 0% to 40% EtOAc in heptane) to give the title compound as a light yellow oil (31.7 g, 85% yield)). m/z 304.1 [M-tBu+H]⁺, ESI pos.

### Step 3: Tert-Butyl (3S,4S)-4-(4-bromophenyl)-3-fluoro-piperidine-1-carboxylate or tert-butyl (3R,4R)-4-(4-bromophenyl)-3-fluoro-piperidine-1-carboxylate

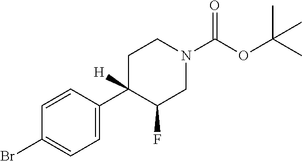 [SMILES: CC(C)(C)OC(=O)N1CC[C@@H](c2ccc(Br)cc2)[C@H](F)C1]

Chiral separation of tert-butyl (3S,4S)-4-(4-bromophenyl)-3-fluoro-piperidine-1-carboxylate and tert-butyl (3R,4R)-4-(4-bromophenyl)-3-fluoro-piperidine-1-carboxylate (31.8 g, 88.8 mmol, Eq: 1.0) by SFC (column: IG, 12 nm, 5 μm, 250×30 mm, eluent: isocratic 5% isopropanol—BPR at 120 bar to 80 g/min) to give the title compound as a colorless oil (13.27 g, 40% yield). m/z 304.0 [M-tBu+H]⁺, ESI pos. The absolute stereochemistry was not determined.

### Step 4: (3S,4S)-4-(4-bromophenyl)-3-fluoro-piperidine hydrochloride or (3R,4R)-4-(4-bromophenyl)-3-fluoro-piperidine Hydrochloride

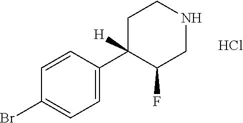 [SMILES: F[C@@H]1CNCC[C@H]1c1ccc(Br)cc1 ; Cl]

To a solution of tert-butyl (3S,4S)-4-(4-bromophenyl)-3-fluoro-piperidine-1-carboxylate or tert-butyl (3R,4R)-4-(4-bromophenyl)-3-fluoro-piperidine-1-carboxylate (10.5 g, 29.3 mmol, Eq: 1.0) in DCM (100 mL) was added HCl (4 M in 1,4-dioxane, 73.3 mL, 293 mmol, Eq: 10). The reaction mixture was stirred for 3 h at rt. The reaction mixture was concentrated in vacuo. The residue was taken up in 40 mL Et₂O and the mixture was stirred for 10 min. The reaction mixture was filtered through sintered glass and washed with Et₂O. The white solid was dried in vacuo to give the title compound (9.5 g, 90% purity, 99% yield). m/z 260.0 [M+H]⁺, ESI pos.

### Step 5: (3S,4S)-4-(4-bromophenyl)-1-ethyl-3-fluoro-piperidine or (3R,4R)-4-(4-bromophenyl)-1-ethyl-3-fluoro-piperidine

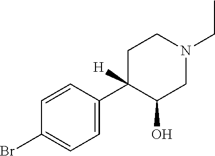 [SMILES: CCN1CC[C@@H](c2ccc(Br)cc2)[C@H](O)C1]

To a suspension of (3S,4S)-4-(4-bromophenyl)-3-fluoro-piperidine hydrochloride or (3R,4R)-4-(4-bromophenyl)-3-fluoro-piperidine hydrochloride (9.50 g, 32.2 mmol, Eq: 1.0) in THF (148 mL) was added NEt₃ (6.53 g, 8.99 mL, 64.5 mmol, Eq: 2.0). Diethyl sulfate (CAS 64-67-5, 5.97 g, 5.07 mL, 38.7 mmol, Eq: 1.2) was added dropwise at rt. The reaction mixture was stirred for 30 min at 35° C. and for 3 h at 55° C. The reaction mixture was poured into EtOAc and washed with Na₂CO₃/water and brine. The organic layer was dried over Na₂SO₄ and concentrated in vacuo. The crude material was purified by flash chromatography (silica gel, 40 g, 1% MeOH in DCM) to give the title compound as a yellow oil (7.20 g, 74% yield). m/z 287.9 [M+H]⁺, ESI pos.

### Step 6: [4-[(3S,4S)-1-ethyl-3-fluoro-4-piperidyl]phenyl]boronic acid or [4-[(3R,4R)-1-ethyl-3-fluoro-4-piperidyl]phenyl]boronic Acid

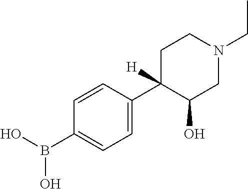 [SMILES: CCN1CC[C@@H](c2ccc(B(O)O)cc2)[C@H](O)C1]

A solution of (3S,4S)-4-(4-bromophenyl)-1-ethyl-3-fluoro-piperidine or (3R,4R)-4-(4-bromophenyl)-1-ethyl-3-fluoro-piperidine (1.000 g, 3.49 mmol, Eq: 1.0) in THF (8.0 mL) was cooled to −76° C. n-Butyllithium (1.6 M in hexanes, 2.4 mL, 3.84 mmol, Eq: 1.1) was added dropwise and the reaction mixture was stirred for 2 h at −76° C. Triethyl borate (618 mg, 0.72 mL, 4.23 mmol, Eq: 1.21) was added at −76° C. and the reaction mixture was stirred for 15 min at −76° C. Then, the dry ice bath was removed and the reaction mixture was stirred at rt (1.5 h). The reaction mixture was quenched with sat. aq. NH₄Cl (10 mL) and stirred for 15 min at rt. The mixture was extracted with EtOAc. The aqueous layer was back-extracted with EtOAc. The organic layers were washed with water and brine. The combined organic layers were dried over Na₂SO₄ and concentrated in vacuo. The crude material was purified by flash chromatography (silica gel, 24 g, 0% to 10% MeOH in DCM) to give the title compound as an off-white solid (744 mg, 90% purity, 76% yield). m/z 252.2 [M+H]⁺, ESI pos.

### Step 7: Ethyl 2-[4-(difluoromethyl)-6-[4-[(3S,4S)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]acetate or ethyl 2-[4-difluoromethyl)-6-[4-[(3R,4R)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]acetate

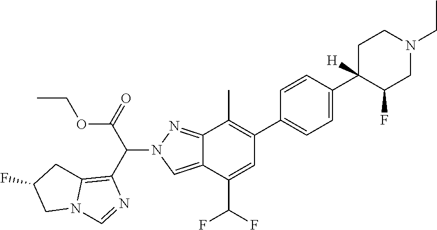 [SMILES: CCOC(=O)C(c1ncn2c1C[C@@H](F)C2)n1cc2c(C(F)F)cc(-c3ccc([C@@H]4CCN(CC)C[C@H]4F)cc3)c(C)c2n1]

A mixture of Intermediate 1 (450 mg, 0.955 mmol, Eq: 1.0), [4-[(3S,4S)-1-ethyl-3-fluoro-4-piperidyl]phenyl]boronic acid or [4-[(3R,4R)-1-ethyl-3-fluoro-4-piperidyl]phenyl]boronic acid (334 mg, 1.2 mmol, Eq: 1.25, 90% purity), Cs₂CO₃ (933 mg, 2.86 mmol, Eq: 3.0), Pd(dppf)Cl₂·CH₂Cl₂ (117 mg, 0.143 mmol, Eq: 0.15) in 1,4-dioxane (7.0 mL) was flushed with argon. The reaction mixture was stirred for 2 h at 100° C. The reaction mixture was cooled to rt and extracted with EtOAc and water. The aqueous layer was back-extracted with EtOAc. The organic layers were washed with water and brine. The combined organic layers were dried over Na₂SO₄ and concentrated in vacuo. The crude material was adsorbed on Isolute HM-N and purified by flash chromatography (silica gel, 24 g, 0% to 100% EtOAc in heptane and then switched to 0% to 10% MeOH in DCM) to give the title compound as a brown foam (587 mg, 98% yield). m/z 598.3 [M+H]⁺, ESI pos.

### Step 8: 2-[4-(difluoromethyl)-6-[4-[(3S,4S)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide or 2-[4-(difluoromethyl)-6-[4-[(3R,4R)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

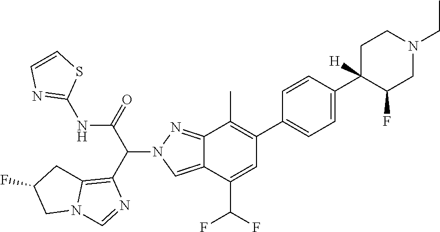 [SMILES: CCN1CC[C@@H](c2ccc(-c3cc(C(F)F)c4cn(C(C(=O)Nc5nccs5)c5ncn6c5C[C@@H](F)C6)nc4c3C)cc2)[C@H](F)C1]

To a solution of ethyl 2-[4-(difluoromethyl)-6-[4-[(3 S,4 S)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]acetate or ethyl 2-[4-(difluoromethyl)-6-[4-[(3R,4R)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]acetate (580 mg, 0.922 mmol, Eq: 1) in MeOH (3.4 mL) and THE (3.4 mL) was added dropwise LiOH (1 M aq., 1.1 mL, 1.1 mmol, Eq: 1.19). The reaction mixture was stirred for 45 min at rt. The reaction mixture was evaporated, twice co-evaporated with toluene and dried under high vacuum. The residue and thiazol-2-amine (111 mg, 1.11 mmol, Eq: 1.2) were suspended in DMF (8.0 mL). DIPEA (370 mg, 0.50 mL, 2.86 mmol, Eq: 3.11) was added followed by HATU (421 mg, 1.11 mmol, Eq: 1.2). The reaction mixture was stirred for 2 h at rt. The reaction mixture was diluted with EtOAc and water. The aqueous layer was back-extracted twice with EtOAc. The organic layers were washed three times with water and once with brine, dried over Na₂SO₄ and concentrated in vacuo. The crude material was adsorbed on Isolute HM-N and purified by flash chromatography (Si-amine, 25 g, 0% to 10% MeOH in EtOAc) to give the title compound as a brown foam (314 mg, 50% yield). m/z 652.3 [M+H]+, ESI pos.

### Example 3

### 2-[4-(Difluoromethyl)-7-methyl-6-[4-[2-(methylamino)ethyl]phenyl]indazol-2-yl]-2-[(6R)-6-fluoro-6,7-dihydro-5H-pyrrolo[1,2-c]imidazol-1-yl]-N-thiazol-2-yl-acetamide Hydrochloride

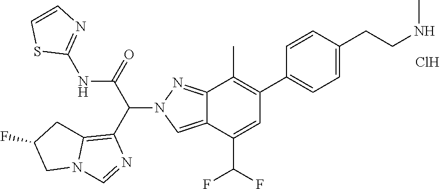 [SMILES: CNCCc1ccc(-c2cc(C(F)F)c3cn(C(C(=O)Nc4nccs4)c4ncn5c4C[C@@H](F)C5)nc3c2C)cc1 ; Cl]

### Step 1: Ethyl 2-(6-(4-(2-((tert-butoxycarbonyl)(methyl)amino)ethyl)phenyl)-4-(difluoromethyl)-7-methyl-2H-indazol-2-yl)-2-((R)-6-fluoro-6,7-dihydro-5H-pyrrolo[1,2-c]imidazol-1-yl)acetate

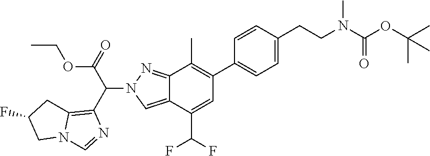 [SMILES: CCOC(=O)C(c1ncn2c1C[C@@H](F)C2)n1cc2c(C(F)F)cc(-c3ccc(CCN(C)C(=O)OC(C)(C)C)cc3)c(C)c2n1]

By analogy with Example 2, Step 7 (100 mg, 212 μmol, Eq: 1.0) and (4-(2-((tert-butoxycarbonyl)(methyl)amino)ethyl)phenyl)boronic acid (CAS 945756-49-0, 71 mg, 255 μmol, Eq: 1.2) were reacted in the presence of Cs₂CO₃ and Pd(dppf)Cl₂·CH₂Cl₂ in dioxane at 105° C. for 1 h to give the title compound as a light brown solid (101 mg, 72% yield). m/z 626.8 [M+H]+, ESI pos.

### Step 2: Tert-Butyl N-[2-[4-[4-(difluoromethyl)-2-[1-[(6R)-6-fluoro-6,7-dihydro-5H-pyrrolo[1,2-c]imidazol-1-yl]-2-oxo-2-(thiazol-2-ylamino)ethyl]-7-methyl-indazol-6-yl]phenyl]ethyl]-N-methyl-carbamate

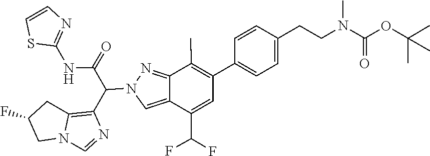 [SMILES: Cc1c(-c2ccc(CCN(C)C(=O)OC(C)(C)C)cc2)cc(C(F)F)c2cn(C(C(=O)Nc3nccs3)c3ncn4c3C[C@@H](F)C4)nc12]

By analogy with Example 2 Step 8, ethyl 2-(6-(4-(2-((tert-butoxycarbonyl)(methyl)amino)ethyl)phenyl)-4-(difluoromethyl)-7-methyl-2H-indazol-2-yl)-2-((R)-6-fluoro-6,7-dihydro-5H-pyrrolo[1,2-c]imidazol-1-yl)acetate (101 mg, 161 μmol, Eq: 1.0) was initially treated with LiOH, and the resulting salt was reacted with 2-aminothiazole in the presence of HATU and DIPEA. Purification by flash chromatography (silica gel, 40 g, 0% to 10% MeOH in EtOAc) gave the title compound as a light brown solid (81 mg, 70% yield). m/z 680.5 [M+H]⁺, ESI pos.

### Step 3: 2-[4-(difluoromethyl)-7-methyl-6-[4-[2-(methylamino)ethyl]phenyl]indazol-2-yl]-2-[(6R)-6-fluoro-6,7-dihydro-5H-pyrrolo[1,2-c]imidazol-1-yl]-N-thiazol-2-yl-acetamide Hydrochloride

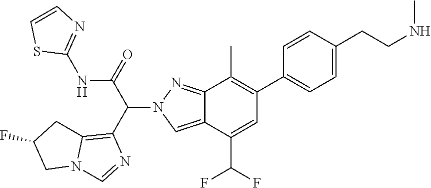 [SMILES: CNCCc1ccc(-c2cc(C(F)F)c3cn(C(C(=O)Nc4nccs4)c4ncn5c4C[C@@H](F)C5)nc3c2C)cc1]

tert-butyl (4-(4-(difluoromethyl)-2-(1-((R)-6-fluoro-6,7-dihydro-5H-pyrrolo[1,2-c]imidazol-1-yl)-2-oxo-2-(thiazol-2-ylamino)ethyl)-7-methyl-2H-indazol-6-yl)phenethyl)(methyl)carbamate (81 mg, 119 μmol, Eq: 1.0) was dissolved in dry dioxane (1 mL) and HCl (4 M in dioxane, 298 μL, 1.19 mmol, Eq: 10). The flask was rotated on a rotary evaporator at rt and pressure, using ultrasonication to aid dissolution. After 2 h, the reaction was concentrated in vacuo to give the title compound as an off-white solid (90 mg, 94% purity, 98% yield). m/z [M+H]⁺ 580.4.

### Example 4

### 2-[4-(Difluoromethyl)-6-[4-[2-(dimethylamino)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

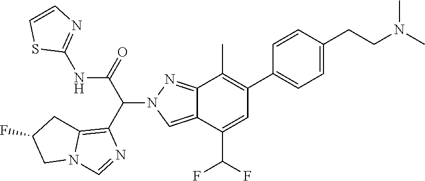 [SMILES: Cc1c(-c2ccc(CCN(C)C)cc2)cc(C(F)F)c2cn(C(C(=O)Nc3nccs3)c3ncn4c3C[C@@H](F)C4)nc12]

To a solution of Example 3 (85 mg, 0.139 mmol, Eq: 1.0) in dioxane (1.0 mL) were added sodium triacetoxyborohydride (294 mg, 1.39 mmol, Eq: 10) and formaldehyde solution (101 mg, 0.093 mL, 1.25 mmol, Eq: 9.0; 37% purity). The reaction mixture was stirred for 72 h at rt. The reaction mixture was quenched with sat. aq. NaHCO₃ and extracted twice with EtOAc. The organic layers were washed with water and brine, dried over Na₂SO₄ and anhydrous MgSO₄ and concentrated in vacuo. The crude material was purified by flash chromatography (Si-amine, 25 g, 0% to 5% MeOH in DCM). The residue was repurified by preparative RP-HPLC to give the title compound as a white solid (16 mg, 18% yield). m/z 594.3 [M+H]⁺, ESI pos.

### Example 5

### 2-[4-(Difluoromethyl)-6-[4-[2-[4-(hydroxymethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

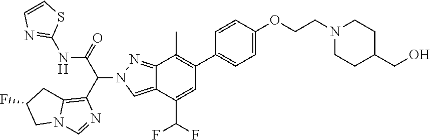 [SMILES: Cc1c(-c2ccc(OCCN3CCC(CO)CC3)cc2)cc(C(F)F)c2cn(C(C(=O)Nc3nccs3)c3ncn4c3C[C@@H](F)C4)nc12]

### Step 1: [1-[2-[4-(4,4,5,5-tetramethyl-1,3,2-dioxaborolan-2-yl)phenoxy]ethyl]-4-piperidyl]methanol

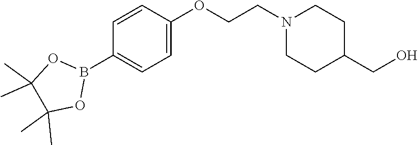 [SMILES: CC1(C)OB(c2ccc(OCCN3CCC(CO)CC3)cc2)OC1(C)C]

To a mixture of [1-[2-(4-bromophenoxy)ethyl]-4-piperidyl]methanol (CAS 1226008-23-6, 1.3 g, 4.14 mmol, Eq: 1.0), bis(pinacolato)diboron (1.16 g, 4.55 mmol, Eq: 1.1), KOAc (1.22 g, 12.4 mmol, Eq: 3.0) in 1,4-dioxane (15 mL) was added Pd(dppf)Cl₂·CH₂Cl₂ (303 mg, 414 μmol, Eq: 0.1). The reaction mixture was flushed with argon and stirred for 3 h at 90° C. The reaction mixture was concentrated in vacuo and purified by flash chromatography (silica gel, 50 g, 0% to 20% MeOH in DCM) to give the title compound as a dark brown oil (1.45 g, 80% purity, 77% yield). m/z 362.2 [M+H]⁺, ESI pos.

### Step 2: Ethyl 2-[4-(difluoromethyl)-6-[4-[2-[4-(hydroxymethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]acetate

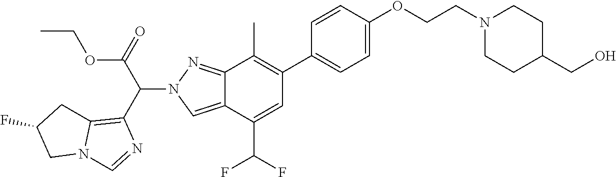 [SMILES: CCOC(=O)C(c1ncn2c1C[C@@H](F)C2)n1cc2c(C(F)F)cc(-c3ccc(OCCN4CCC(CO)CC4)cc3)c(C)c2n1]

By analogy with Example 2 Step 7, Intermediate 1 (150 mg, 318 μmol, Eq: 1.0) and [1-[2-[4-(4,4,5,5-tetramethyl-1,3,2-dioxaborolan-2-yl)phenoxy]ethyl]-4-piperidyl]methanol (149 mg, 414 μmol Eq: 1.3) were reacted in the presence of Cs₂CO₃ and Pd(dppf)Cl₂·CH₂Cl₂ for 2.5 h at 70° C. Purification by flash chromatography (Si-Amine, 25 g, 0% to 10% MeOH in EtOAc) gave the title compound as a brown gum (67.4 mg, 68.9 μmol, 64% purity, 21.7% yield). m/z 626.4 [M+H]⁺, ESI pos.

### Step 3: 2-[4-(difluoromethyl)-6-[4-[2-[4-(hydroxymethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

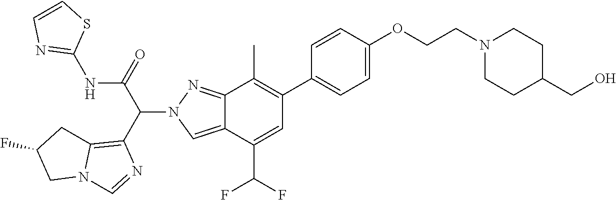 [SMILES: Cc1c(-c2ccc(OCCN3CCC(CO)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[2-[4-(hydroxymethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]acetate (67.4 mg, 68.9 μmol, Eq: 1.0) was initially treated with LiOH, and the resulting salt was reacted with 2-aminothiazole in the presence of HATU and DIPEA. Purification by flash chromatography (Si-Amine, 12 g, 0% to 20% MeOH in EtOAc) gave the title compound as a light brown solid (54 mg, 75% yield). m/z 340.8 [M+H]⁺, ESI pos.

### Example 6

### 2-[4-(Difluoromethyl)-6-[4-[[4-(hydroxymethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

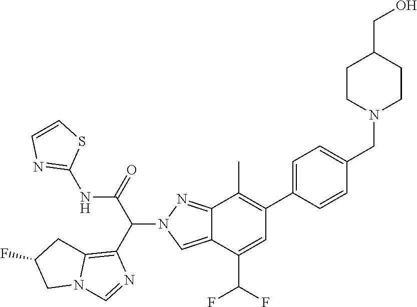 [SMILES: Cc1c(-c2ccc(CN3CCC(CO)CC3)cc2)cc(C(F)F)c2cn(C(C(=O)Nc3nccs3)c3ncn4c3C[C@@H](F)C4)nc12]

### Step 1: [1-[(4-bromophenyl)methyl]-4-piperidyl]methanol

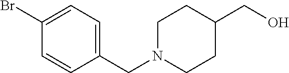 [SMILES: OCC1CCN(Cc2ccc(Br)cc2)CC1]

In a 250 mL round-bottomed flask, piperidin-4-ylmethanol (1.49 g, 13 mmol, Eq: 1.2) was combined with DCM (60 mL) to give a colorless solution. 4-Bromobenzaldehyde (2 g, 10.8 mmol, Eq: 1.0) and sodium triacetoxyborohydride (2.75 g, 13 mmol, Eq: 1.2) were added at 0° C. The ice-bath was removed and the reaction mixture was stirred at rt for 3 h. The reaction mixture was poured into sat. aq. NaHCO₃ and extracted with DCM (2×). The crude material was purified by flash chromatography (silica gel, 40 g, 0% to 100% (4:1 DCM/MeOH+0.5% NH₄OH) in DCM) to give title compound as a colorless liquid (2.90 g, 95% purity, 89% yield). m/z 284.1 [M+H]⁺, ESI pos.

### Step 2: [1-[[4-(4,4,5,5-tetramethyl-1,3,2-dioxaborolan-2-yl)phenyl]methyl]-4-piperidyl]methanol

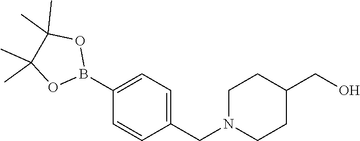 [SMILES: CC1(C)OB(c2ccc(CN3CCC(CO)CC3)cc2)OC1(C)C]

By analogy with Example 5 Step 1, [1-[(4-bromophenyl)methyl]-4-piperidyl]methanol (2.9 g, 10.2 mmol, Eq: 1.0), was reacted with bis(pinacolato)diboron in the presence of KOAc and Pd(dppf)Cl₂·CH₂Cl₂ at 90° C. for 2 h. Purification by flash chromatography (Si-amine, 40 g, then a second with 25 g, 0% to 80% (4:1 EtOAc/MeOH) in EtOAc)) gave the title compound as a colorless oil (3.60 g, 85% purity, 90% yield). m/z 332.3 [M+H]+, ESI pos.

### Step 3: Ethyl 2-[4-(difluoromethyl)-6-[4-[[4-(hydroxymethyl)-1-piperidyl]methyl]phenyl]-7-methyl-indazol-2-yl]-2-[(6R)-6-fluoro-6,7-dihydro-5H-pyrrolo[1,2-c]imidazol-1-yl]acetate

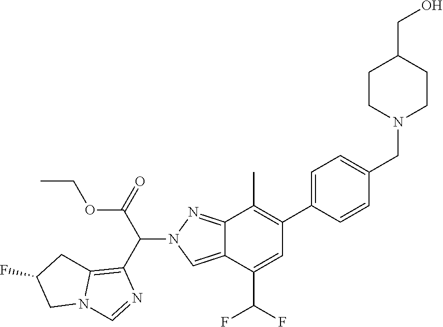 [SMILES: CCOC(=O)C(c1ncn2c1C[C@@H](F)C2)n1cc2c(C(F)F)cc(-c3ccc(CN4CCC(CO)CC4)cc3)c(C)c2n1]

By analogy with Example 2 Step 7, Intermediate 1 (100 mg, 212 μmol, Eq: 1.0) and [1-[[4-(4,4,5,5-tetramethyl-1,3,2-dioxaborolan-2-yl)phenyl]methyl]-4-piperidyl]methanol (91.4 mg, 276 μmol, Eq: 1.3) were reacted in the presence of Cs₂CO₃ and Pd(dppf)Cl₂·CH₂Cl₂ at 70° C. for 2.5 h. Purification by flash chromatography (silica gel, 25 g, 0% to 10% MeOH in DCM) gave the title compound as a brown oil (105 mg, 89% purity, 83% yield). m/z 596.3 [M+H]⁺, ESI pos.

### Step 4: 2-[4-(difluoromethyl)-6-[4-[[4-(hydroxymethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

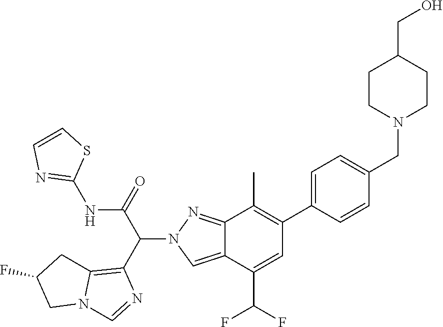 [SMILES: Cc1c(-c2ccc(CN3CCC(CO)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[[4-(hydroxymethyl)-1-piperidyl]methyl]phenyl]-7-methyl-indazol-2-yl]-2-[(6R)-6-fluoro-6,7-dihydro-5H-pyrrolo[1,2-c]imidazol-1-yl]acetate (100 mg, 168 μmol, Eq: 1.0) was initially treated with LiOH, and the resulting salt was reacted with 2-aminothiazole in the presence of HATU and DIPEA. Purification by flash chromatography (silica gel, 12 g, 0% to 100% (DCM:MeOH:NH₄OH 9:1:0.05) in DCM) gave the title compound as a light brown foam (55 mg, 90% purity, 45% yield). m/z 650.3 [M+H]⁺, ESI pos.

### Example 7

### 2-[4-(Difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

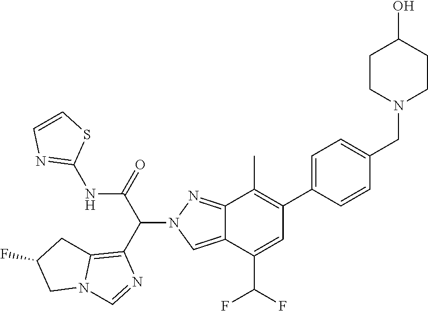 [SMILES: Cc1c(-c2ccc(CN3CCC(O)CC3)cc2)cc(C(F)F)c2cn(C(C(=O)Nc3nccs3)c3ncn4c3C[C@@H](F)C4)nc12]

### Step 1: 1-[(4-bromophenyl)methyl]piperidin-4-ol

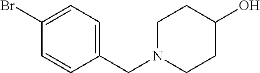 [SMILES: OC1CCN(Cc2ccc(Br)cc2)CC1]

4-Bromobenzaldehyde (1.5 g, 8.11 mmol, Eq: 1) and piperidin-4-ol (1.07 g, 10.5 mmol, Eq: 1.3) were dissolved in DCM (10 mL). Then, sodium triacetoxyborohydride (5.15 g, 24.3 mmol, Eq: 3.0) was added, followed by stirring at rt for 16 h. The reaction mixture was diluted with sat. aq. Na₂CO₃ and extracted twice with EtOAc. The organic layers were washed with water and brine, dried over Na₂SO₄, filtered and concentrated in vacuo. The crude material was purified by flash chromatography (silica gel, 80 g, 0% to 20% MeOH in DCM) to give the title compound as a colorless oil (2.11 g, 96% yield). m/z 271.9 [M+H]⁺, ESI pos.

### Step 2: 1-[[4-(4,4,5,5-tetramethyl-1,3,2-dioxaborolan-2-yl)phenyl]methyl]piperidin-4-ol)

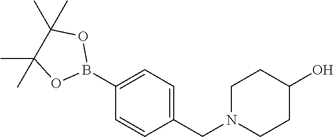 [SMILES: CC1(C)OB(c2ccc(CN3CCC(O)CC3)cc2)OC1(C)C]

By analogy with Example 5 Step 1, 1-[(4-bromophenyl)methyl]piperidin-4-ol (2.11 g, 7.81 mmol, Eq: 1.0) was reacted with bis(pinacolato)diboron in the presence of KOAc and Pd(dppf)Cl₂·CH₂Cl₂ at 90° C. for 2 h. Purification by flash chromatography (Si-amine, 40 g, then again 40 g, 0% to 80% (4:1 EtOAc-MeOH) in EtOAc) gave the title compound as a colorless oil (2.28 g, 75% purity, 96% yield). m/z 317.9 [M+H]⁺, ESI pos.

### Step 3: Ethyl 2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]acetate

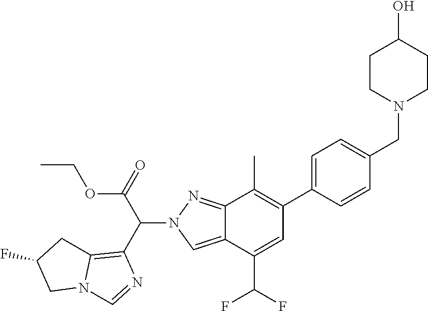 [SMILES: CCOC(=O)C(c1ncn2c1C[C@@H](F)C2)n1cc2c(C(F)F)cc(-c3ccc(CN4CCC(O)CC4)cc3)c(C)c2n1]

By analogy with Example 2 Step 7, Intermediate 1 (100 mg, 212 μmol, Eq: 1.0) and 1-[[4-(4,4,5,5-tetramethyl-1,3,2-dioxaborolan-2-yl)phenyl]methyl]piperidin-4-01) (117 mg, 75% purity, 276 μmol Eq: 1.3) were reacted in the presence of Cs₂CO₃ and Pd(dppf)Cl₂·CH₂Cl₂ at 70° C. for 2.5 h. Purification by flash chromatography (silica gel, 25 g, 0% to 10% MeOH in DCM) gave the title compound as a colorless oil (60 mg, 73% purity, 48% yield). m/z 582.3 [M+H]⁺, ESI pos.

### Step 4: 2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

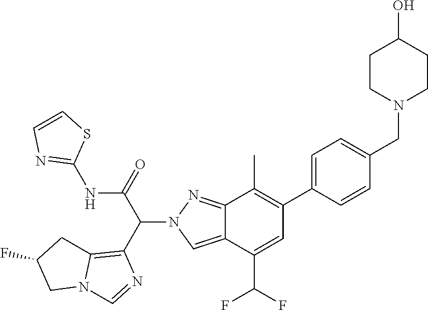 [SMILES: Cc1c(-c2ccc(CN3CCC(O)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]acetate (0.06 g, 103 μmol, Eq: 1.0) was initially treated with LiOH, and the resulting salt was reacted with 2-amiothiazole in the presence of HATU and DIPEA. Purification by flash chromatography (silica gel, 12 g, 0% to 100% (DCM:MeOH:NH₄OH 9:1:0.05) in DCM) gave the title compound as a colorless oil (60 mg, 73% purity, 48% yield). m/z 636.3 [M+H]⁺, ESI pos.

### Example 8

### 2-[4-(Difluoromethyl)-7-methyl-6-(4-morpholinophenyl)indazol-2-yl]-2-(6,7-dihydro-5H-pyrrolo[1,2-c]imidazol-1-yl)-N-thiazol-2-yl-acetamide

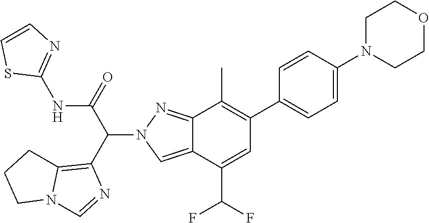 [SMILES: Cc1c(-c2ccc(N3CCOCC3)cc2)cc(C(F)F)c2cn(C(C(=O)Nc3nccs3)c3ncn4c3CCC4)nc12]

### Step 1: ethyl 2-[4-(difluoromethyl)-7-methyl-6-(4-morpholinophenyl)indazol-2-yl]-2-(6,7-dihydro-5H-pyrrolo[1,2-c]imidazol-1-yl)acetate

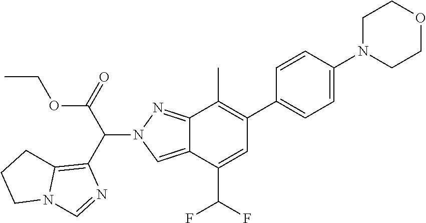 [SMILES: CCOC(=O)C(c1ncn2c1CCC2)n1cc2c(C(F)F)cc(-c3ccc(N4CCOCC4)cc3)c(C)c2n1]

Intermediate 2 (120 mg, 265 μmol, Eq: 1.0), (4-morpholinophenyl)boronic acid (66.3 mg, 320 μmol, Eq: 1.21), Pd(dppf)Cl₂·CH₂Cl₂ (22.2 mg, 29.1 μmol, Eq: 0.11) and Cs₂CO₃ (259 mg, 794 μmol, Eq: 3.0) were mixed in a sealed tube, and dissolved in 1.6 mL of dry dioxane. Ar gas was bubbled through the mixture while sonicating. The reaction was heated at 100° C. for 2 h, then cooled to rt, diluted with EtOAc, washed with sat. aq. NaHCO₃ and back-extracted two times with EtOAc. The organic layers were washed with water and brine, dried over Na₂SO₄, filtered and evaporated to dryness. The crude material was purified by flash chromatography (silica gel, 12 g, 0% to 10% MeOH in EtOAc) to give the title compound as a yellow oil (105 mg, 74% yield). m/z 536.2 [M+H]⁺, ESI pos.

### Step 2: 2-[4-(difluoromethyl)-7-methyl-6-(4-morpholinophenyl)indazol-2-yl]-2-(6,7-dihydro-5H-pyrrolo[1,2-c]imidazol-1-yl)-N-thiazol-2-yl-acetamide

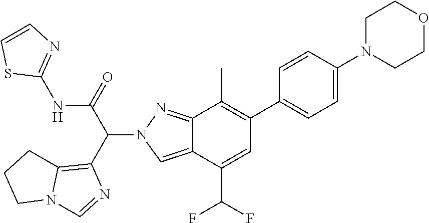 [SMILES: Cc1c(-c2ccc(N3CCOCC3)cc2)cc(C(F)F)c2cn(C(C(=O)Nc3nccs3)c3ncn4c3CCC4)nc12]

Ethyl 2-[4-(difluoromethyl)-7-methyl-6-(4-morpholinophenyl)indazol-2-yl]-2-(6,7-dihydro-5H-pyrrolo[1,2-c]imidazol-1-yl)acetate (0.1 g, 187 μmol, Eq: 1.0) was dissolved in 1 mL THE and 1 mL MeOH. LiOH (1 M aq., 224 μL, 224 μmol, Eq: 1.2) was added dropwise. The reaction mixture was stirred at rt for 45 min (a precipitate was formed). The reaction mixture was carefully neutralized with HCl (1 M aq., 224 μL, 224 μmol, Eq: 1.2) (pH-6) and concentrated, then co-evaporated twice from toluene and dried under high vacuum. The residue and 2-aminothiazole (28 mg, 280 μmol, Eq: 1.5) were suspended in 1 mL DMF. DIPEA (72.4 mg, 97.8 μL, 560 μmol, Eq: 3.0) was added followed by HATU (92.3 mg, 243 μmol, Eq: 1.3). The reaction mixture was stirred at rt for 2 h. The reaction mixture was diluted with EtOAc and water. The aqueous layer was back-extracted with EtOAc. The organic layers were washed three times with water and once with brine, dried over Na₂SO₄, filtered and concentrated. The residue was adsorbed on Isolute HM-N and purified by flash chromatography (silica gel, 12 g, EtOAc then 0% to 5% MeOH in DCM) to give the title compound as a light yellow solid (60 mg, 90% purity, 54% yield). m/z 590.2 [M+H]⁺, ESI pos.

### Example 9

### 2-[4-(Difluoromethyl)-6-[4-[2-[4-(hydroxymethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

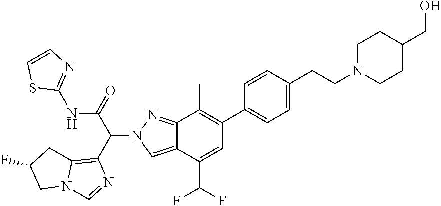 [SMILES: Cc1c(-c2ccc(CCN3CCC(CO)CC3)cc2)cc(C(F)F)c2cn(C(C(=O)Nc3nccs3)c3ncn4c3C[C@@H](F)C4)nc12]

### Step 1: [1-[2-(4-bromophenyl)ethyl]-4-piperidyl]methanol

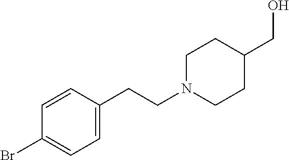 [SMILES: OCC1CCN(CCc2ccc(Br)cc2)CC1]

2-(4-Bromophenyl)acetaldehyde (2 g, 10 mmol, Eq: 1.0) and piperidin-4-ylmethanol (1.5 g, 13.1 mmol, Eq: 1.3) were dissolved in DCM (20 mL). Then, sodium triacetoxyborohydride (6.39 g, 30.1 mmol, Eq: 3.0) was added, followed by stirring at rt for 16 h. The reaction mixture was washed with sat. aq. NaHCO₃ and extracted two times with DCM. The organic layers were washed with water, dried over Na₂SO₄, filtered and concentrated in vacuo. The crude material was purified by flash chromatography (silica gel, 80 g, 0% to 100% (DCM:MeOH:NH₄OH 9:1:0.5) in DCM). The pure fractions were combined and concentrated in vacuo to give the title compound as a colorless liquid (2.16 g, 72% yield). m/z 300.1 [M+H]⁺, ESI pos.

### Step 2: [1-[2-[4-(4,4,5,5-tetramethyl-1,3,2-dioxaborolan-2-yl)phenyl]ethyl]-4-piperidyl]methanol

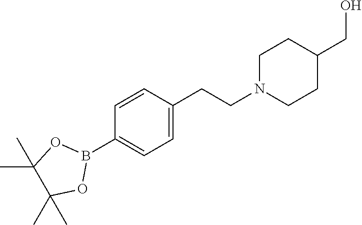 [SMILES: CC1(C)OB(c2ccc(CCN3CCC(CO)CC3)cc2)OC1(C)C]

By analogy with Example 5 Step 1, [1-[2-(4-bromophenyl)ethyl]-4-piperidyl]methanol (2.16 g, 7.24 mmol, Eq: 1.0) was reacted with bis(pinacolato)diboron in the presence of KOAc and Pd(dppf)Cl₂·CH₂Cl₂ at 90° C. for 4 h. Purification by flash chromatography (silica gel, 40 g, 0% to 10% MeOH in DCM) gave the title compound as a dark brown oil (3.60 g, 60% purity, 86% yield). m/z 345.9 [M+H]⁺, ESI pos.

### Step 3: ethyl 2-[4-(difluoromethyl)-6-[4-[2-[4-(hydroxymethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]acetate

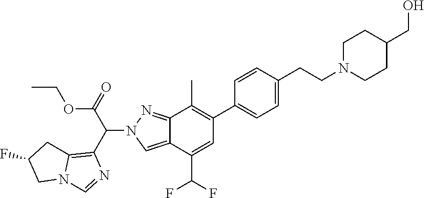 [SMILES: CCOC(=O)C(c1ncn2c1C[C@@H](F)C2)n1cc2c(C(F)F)cc(-c3ccc(CCN4CCC(CO)CC4)cc3)c(C)c2n1]

By analogy with Example 2 Step 7, Intermediate 1 (100 mg, 212 μmol, Eq: 1.0) and [1-[2-[4-(4,4,5,5-tetramethyl-1,3,2-dioxaborolan-2-yl)phenyl]ethyl]-4-piperidyl]methanol were reacted in the presence of Cs₂CO₃ and Pd(dppf)Cl₂·CH₂Cl₂ at 70° C. for 2.5 h. Purification by flash chromatography (silica gel, 25 g, 0% to 100% (DCM:MeOH:NH₄OH 9:1:0.05) in DCM) gave the title compound as a brown solid (85 mg, 65% yield). m/z 610.3 [M+H]⁺, ESI pos.

### Step 4: 2-[4-(difluoromethyl)-6-[4-[2-[4-(hydroxymethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

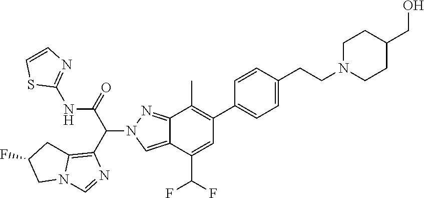 [SMILES: Cc1c(-c2ccc(CCN3CCC(CO)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[2-[4-(hydroxymethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]acetate (85 mg, 139 μmol, Eq: 1.0) was initially treated with LiOH, and the resulting salt was reacted with 2-aminothiazole in the presence of HATU and DIPEA. Purification by flash chromatography (silica gel, 12 g, 0% to 100% (DCM:MeOH:NH₄OH 9:1:0.05) in DCM) gave the title compound as a light brown foam (35 mg, 90% purity, 34% yield). m/z 664.3 [M+H]⁺, ESI pos.

### Example 10

### 2-[4-(Difluoromethyl)-6-[4-[2-(4-hydroxy-1-piperidyl)ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

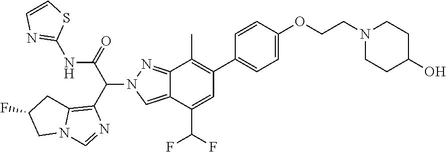 [SMILES: Cc1c(-c2ccc(OCCN3CCC(O)CC3)cc2)cc(C(F)F)c2cn(C(C(=O)Nc3nccs3)c3ncn4c3C[C@@H](F)C4)nc12]

### Step 1: 1-[2-[4-(4,4,5,5-tetramethyl-1,3,2-dioxaborolan-2-yl)phenoxy]ethyl]piperidin-4-ol

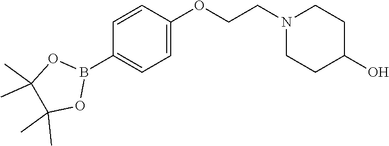 [SMILES: CC1(C)OB(c2ccc(OCCN3CCC(O)CC3)cc2)OC1(C)C]

By analogy with Example 5 Step 1, 1-[2-(4-bromophenoxy)ethyl]piperidin-4-ol (CAS 1226008 23-6, 708 mg, 2.12 mmol, Eq: 1), was reacted with bis(pinacolato)diboron in the presence of KOAc and Pd(dppf)Cl₂·CH₂Cl₂ for 3 h at 90° C. Purification by flash chromatography (silica gel, 25 g, 0% to 20% MeOH in DCM) gave the title compound as a brown oil (537 mg, 79% yield). m/z 348.3 [M+H]+, ESI pos.

### Step 2: Ethyl 2-[4-(difluoromethyl)-6-[4-[2-(4-hydroxycyclohexyl)ethoxy]phenyl]-7-methyl-indazol-2-yl]-2-[(6R)-6-fluoro-6,7-dihydro-5H-pyrrolo[1,2-c]imidazol-1-yl]acetate

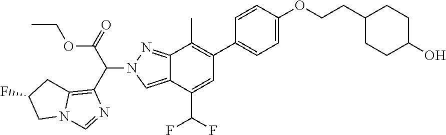 [SMILES: CCOC(=O)C(c1ncn2c1C[C@@H](F)C2)n1cc2c(C(F)F)cc(-c3ccc(OCCC4CCC(O)CC4)cc3)c(C)c2n1]

By analogy with Example 2 Step 7, Intermediate 1 (100 mg, 212 μmol, Eq: 1.0) and 1-[2-[4-(4,4,5,5-tetramethyl-1,3,2-dioxaborolan-2-yl)phenoxy]ethyl]piperidin-4-ol (96 mg, 276 μmol, Eq: 1.3) were reacted in the presence of Cs₂CO₃ and Pd(dppf)Cl₂·CH₂Cl₂ for 2.5 h at 70° C. Purification by flash chromatography (Si-Amine, 25 g, 0% to 10% MeOH in EtOAc) gave the title compound as a brown oil (123 mg, 76% purity, 72% yield). m/z 306.8 [M+H]⁺, ESI pos.

### Step 3: 2-[4-(difluoromethyl)-6-[4-[2-(4-hydroxy-1-piperidyl)ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

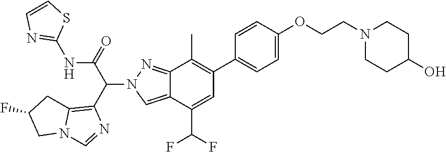 [SMILES: Cc1c(-c2ccc(OCCN3CCC(O)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[2-(4-hydroxycyclohexyl)ethoxy]phenyl]-7-methyl-indazol-2-yl]-2-[(6R)-6-fluoro-6,7-dihydro-5H-pyrrolo[1,2-c]imidazol-1-yl]acetate (123 mg, 200 μcool, Eq: 1.0) was initially treated with LiOH, and the resultant salt was reacted with thiazol-2-amine in the presence of HATU and DIPEA. Purification by flash chromatography (silica gel, 24 g, 0% to 20% MeOH in DCM) gave the title compound as a brown solid (36 mg, 27% yield). m/z 334.0 [M+H]+, ESI pos.

### Example 11

### 2-[4-(Difluoromethyl)-6-[4-(4-hydroxy-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

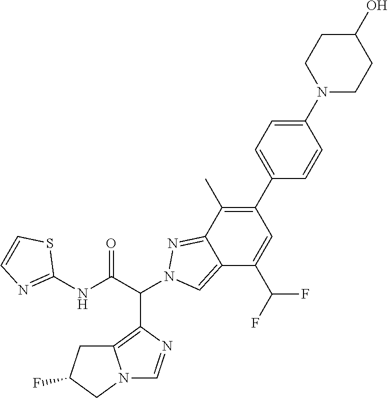 [SMILES: Cc1c(-c2ccc(N3CCC(O)CC3)cc2)cc(C(F)F)c2cn(C(C(=O)Nc3nccs3)c3ncn4c3C[C@@H](F)C4)nc12]

### Step 1: [1-(4-bromophenyl)-4-piperidyl]oxy-tert-butyl-dimethyl-silane

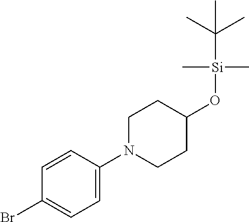 [SMILES: CC(C)(C)[Si](C)(C)OC1CCN(c2ccc(Br)cc2)CC1]

To a solution of 1-(4-bromophenyl)piperidin-4-ol (CAS 1226154-84-2, 300 mg, 1.17 mmol, Eq: 1.0) in DCM (5 mL) was added imidazole (319 mg, 4.68 mmol, Eq: 4.0) and tert-butyldimethylsilyl chloride (441 mg, 2.93 mmol, Eq: 2.5) and stirred at rt. After 1 h, the reaction mixture was diluted with DCM, and washed with water, sat. aq. NaHCO₃ and brine. The aqueous layers were back-extracted with DCM. The combined organic layers were dried over MgSO₄ and concentrated under reduced pressure. The title compound was obtained as an off-white solid (431 mg, 99% yield). m/z 372.1 [M+H]⁺, ESI pos.

### Step 2: Tert-butyl-dimethyl-[[1-[4-(4,4,5,5-tetramethyl-1,3,2-dioxaborolan-2-yl)phenyl]-4-piperidyl]oxy]silane

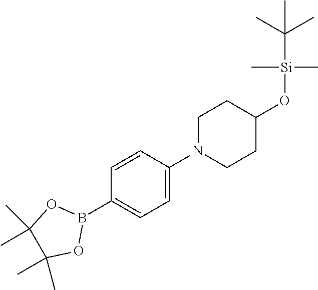

By analogy with Example 5 Step 1, [1-(4-bromophenyl)-4-piperidyl]oxy-tert-butyl-dimethyl-silane (280 mg, 756 μmol, Eq: 1.0), was reacted with bis(pinacolato)diboron in the presence of KOAc and Pd(dppf)Cl₂·CH₂Cl₂ at 90° C. for 3 h. Purification by flash chromatography (silica gel. 20 g, 0% to 20% EtOAc in heptane) gave the title compound as a white solid (264 mg, 85% purity, 71% yield). m/z 418.1 [M+H]⁺, ESI pos.

### Step 3: Ethyl 2-[4-(difluoromethyl)-7-methyl-6-[4-(4-tert-butyl-dimethylsilyloxy-1-piperidyl)phenyl]indazol-2-yl]-2-[(6R)-6-fluoro-6,7-dihydro-5H-pyrrolo[1,2-c]imidazol-1-yl]acetate

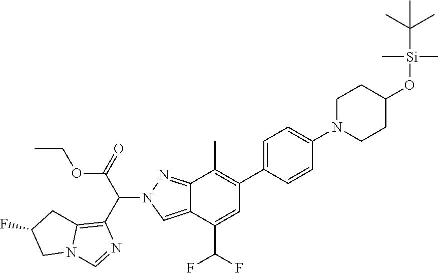 [SMILES: CCOC(=O)C(c1ncn2c1C[C@@H](F)C2)n1cc2c(C(F)F)cc(-c3ccc(N4CCC(O[Si](C)(C)C(C)(C)C)CC4)cc3)c(C)c2n1]

By analogy with Example 2 Step 7, Intermediate 1 (100 mg, 212 μmol, Eq: 1.0) and 4-((tert-butyldimethylsilyl)oxy)-1-(4-(4,4,5,5-tetramethyl-1,3,2-dioxaborolan-2-yl)phenyl)piperidine (156 mg, 318 μmol, Eq: 1.5) were reacted in the presence of Cs₂CO₃ and Pd(dppf)Cl₂·CH₂Cl₂ at 65° C. for 1.5 h. Purification by flash chromatography (silica gel, 24 g, 0% to 10% MeOH in EtOAc) gave the title compound as a white solid (138 mg, 70% purity, 95% yield). m/z 682.5 [M+H]⁺, ESI pos.

### Step 4: 2-[4-(difluoromethyl)-7-methyl-6-[4-(4-tert-butyl-dim ethyl silyloxy-1-piperidyl)phenyl]indazol-2-yl]-2-[(6R)-6-fluoro-6,7-dihydro-5H-pyrrolo[1,2-c]imidazol-1-yl]-N-thiazol-2-yl-acetamide

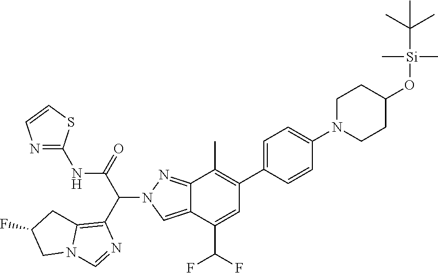 [SMILES: Cc1c(-c2ccc(N3CCC(O[Si](C)(C)C(C)(C)C)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-7-methyl-6-[4-(4-tert-butyl-dim ethyl silyloxy-1-piperidyl)phenyl]indazol-2-yl]-2-[(6R)-6-fluoro-6,7-dihydro-5H-pyrrolo[1,2-c]imidazol-1-yl]acetate was initially treated with LiOH, and the resultant salt was reacted with 2-aminothiazole in the presence of HATU and DIPEA. Purification by flash chromatography (silica gel, 12 g, 0% to 100% (DCM:MeOH:NH₄OH 9:1:0.05) in DCM) gave the title compound as a yellow oil (55 mg, 80% purity, 45% yield). m/z 736.4 [M+H]⁺, ESI pos.

### Step 5: 2-[4-(difluoromethyl)-6-[4-(4-hydroxy-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

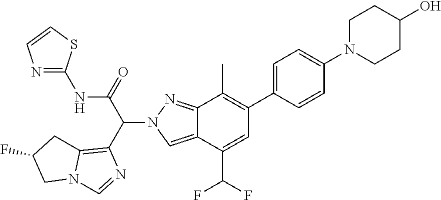 [SMILES: Cc1c(-c2ccc(N3CCC(O)CC3)cc2)cc(C(F)F)c2cn(C(C(=O)Nc3nccs3)c3ncn4c3C[C@@H](F)C4)nc12]

2-[4-(Difluoromethyl)-7-methyl-6-[4-(4-tert-butyl-dim ethyl silyloxy-1-piperidyl)phenyl]indazol-2-yl]-2-[(6R)-6-fluoro-6,7-dihydro-5H-pyrrolo[1,2-c]imidazol-1-yl]-N-thiazol-2-yl-acetamide was dissolved in THF (2.3 mL) and TBAF (1 M in THF, 149 μL, 149 μmol, Eq: 2.0) was added at rt. The mixture was stirred for 16 h at rt. The reaction mixture was diluted with sat. aq. NaHCO₃ and extracted two times with EtOAc. The organic layers were washed with a mixture of water and brine then with brine, dried over Na₂SO₄, filtered and evaporated to dryness. The crude material was purified by flash chromatography (silica gel, 12 g, 0% to 100% (DCM:MeOH:NH₄OH 9:1:0.05) in DCM) to give the title compound as a light yellow foam (25 mg, 51% yield). m/z 622.2 [M+H]⁺, ESI pos.

### Example 12

### 2-[4-(Difluoromethyl)-6-[4-[[4-(1-hydroxyethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

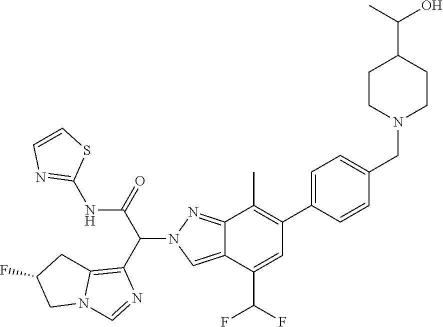 [SMILES: Cc1c(-c2ccc(CN3CCC(C(C)O)CC3)cc2)cc(C(F)F)c2cn(C(C(=O)Nc3nccs3)c3ncn4c3C[C@@H](F)C4)nc12]

### Step 1: 1-[1-[(4-bromophenyl)methyl]-4-piperidyl]ethanol

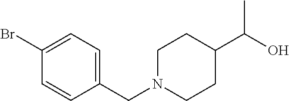 [SMILES: CC(O)C1CCN(Cc2ccc(Br)cc2)CC1]

4-Bromobenzaldehyde (1 g, 5.4 mmol, Eq: 1.0) and 1-(piperidin-4-yl)ethan-1-ol (CAS 6457-48-3, 908 mg, 7.03 mmol, Eq: 1.3) were dissolved in DCM (10 mL). Then, sodium triacetoxyborohydride (1.72 g, 8.11 mmol, Eq: 1.5) was added, followed by stirring at rt for 16 h. The reaction mixture was diluted with sat. aq. Na₂CO₃ and extracted two times with EtOAc. The organic layers were washed with water and brine, dried over Na₂SO₄, filtered and concentrated in vacuo. The crude material was purified by flash chromatography (silica gel, 80 g, 0% to 20% MeOH in DCM). Fractions containing product were combined and concentrated to give the title compound as a light yellow foam (1.15 g, 71% yield). m/z 299.8 [M+H]⁺, ESI pos.

### Step 2: 1-[1-[[4-(4,4,5,5-tetramethyl-1,3,2-dioxaborolan-2-yl)phenyl]methyl]-4-piperidyl]ethanol

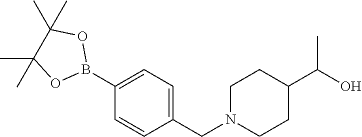 [SMILES: CC(O)C1CCN(Cc2ccc(B3OC(C)(C)C(C)(C)O3)cc2)CC1]

By analogy with Example 5 Step 1, 1-[1-[(4-bromophenyl)methyl]-4-piperidyl]ethanol (1.5 g, 5.03 mmol, Eq: 1.0) was reacted with bis(pinacolato)diboron in the presence of KOAc and Pd(dppf)Cl₂·CH₂Cl₂ at 90° C. for 2 h. Purification by flash chromatography (silica gel, 24 g, 0% to 10% MeOH in DCM) gave the title compound as a dark brown solid (850 mg, 48% yield). m/z 346.1 [M+H]⁺, ESI pos.

### Step 3: Ethyl 2-[4-(difluoromethyl)-6-[4-[[4-(1-hydroxyethyl)-1-piperidyl]methyl]phenyl]-7-methyl-indazol-2-yl]-2-[(6R)-6-fluoro-6,7-dihydro-5H-pyrrolo[1,2-c]imidazol-1-yl]acetate

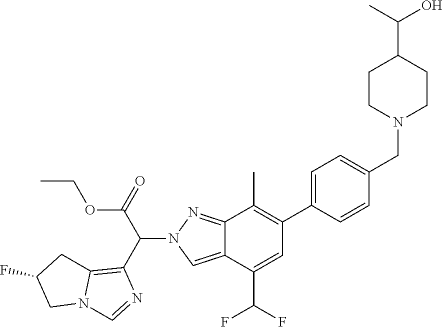 [SMILES: CCOC(=O)C(c1ncn2c1C[C@@H](F)C2)n1cc2c(C(F)F)cc(-c3ccc(CN4CCC(C(C)O)CC4)cc3)c(C)c2n1]

By analogy with Example 2 Step 7, Intermediate 1 (100 mg, 212 μmol, Eq: 1.0) and 1-[1-[[4-(4,4,5,5-tetramethyl-1,3,2-dioxaborolan-2-yl)phenyl]methyl]-4-piperidyl]ethanol (112 mg, 85% purity, 276 μmol, Eq: 1.3) were reacted in the presence of Cs₂CO₃ and Pd(dppf)Cl₂·CH₂Cl₂ at 70° C. for 2.5 h. Purification by flash chromatography (silica gel, 25 g, 0% to 100% (DCM:MeOH:NH₄OH 9:1:0.05) in DCM) gave the title compound as a light brown oil (56 mg, 72% purity, 43% yield). m/z 610.3 [M+H]⁺, ESI pos.

### Step 4: 2-[4-(difluoromethyl)-6-[4-[[4-(1-hydroxyethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

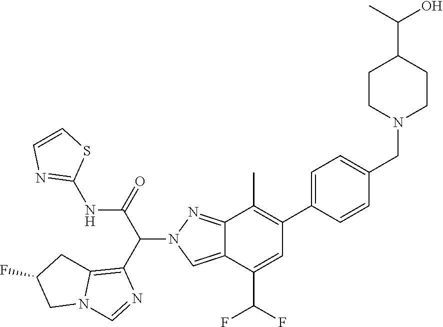 [SMILES: Cc1c(-c2ccc(CN3CCC(C(C)O)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[[4-(1-hydroxyethyl)-1-piperidyl]methyl]phenyl]-7-methyl-indazol-2-yl]-2-[(6R)-6-fluoro-6,7-dihydro-5H-pyrrolo[1,2-c]imidazol-1-yl]acetate was first treated with LiOH, and the resultant salt was reacted with 2-aminothiazole in the presence of HATU and DIPEA. Purification by flash chromatography (silica gel, 12 g, 0% to 100% (DCM:MeOH:NH₄OH 9:1:0.05) in DCM) gave the title compound as a light brown foam (45 mg, 65% yield). m/z 664.3 [M+H]⁺, ESI pos.

### Example 13

### 2-[6-[4-(1,4-Diazabicyclo[3.2.1]octan-4-yl)phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

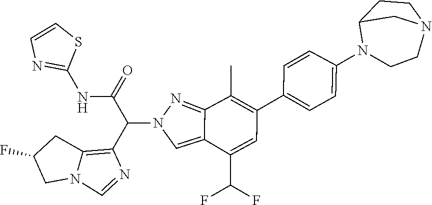 [SMILES: Cc1c(-c2ccc(N3CCN4CCC3C4)cc2)cc(C(F)F)c2cn(C(C(=O)Nc3nccs3)c3ncn4c3C[C@@H](F)C4)nc12]

### Step 1: 4-(4-bromophenyl)-1,4-diazabicyclo[3.2.1]octane

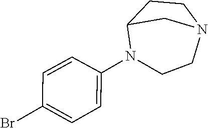 [SMILES: Brc1ccc(N2CCN3CCC2C3)cc1]

1-Bromo-4-iodobenzene (1.88 g, 6.64 mmol, Eq: 1.3), 1,4-diazabicyclo[3.2.1]octane dihydrochloride (CAS 5492-61-5, 945 mg, 5.11 mmol, Eq: 1.0), NaO^{t}Bu (2.45 g, 25.5 mmol, Eq: 5.0), xantphos (369 mg, 638 μmol, Eq: 0.125) and Pd₂(dba)₃ (234 mg, 255 μmol, Eq: 0.05) were dissolved in toluene (10 mL), followed by stirring at 85° C. for 1 h. The reaction mixture was diluted with water and extracted two times with EtOAc. The organic layers were washed with water and brine, dried over Na₂SO₄, filtered and concentrated in vacuo. The crude material was purified by flash chromatography (silica gel, 40 g, 0% to 100% (DCM:MeOH:NH₄OH 9:1:0.05) in DCM) to give the title compound as a light yellow solid (596 mg, 90% purity, 39% yield). m/z 268.7 [M+H]⁺, ESI pos.

### Step 2: 4-[4-(4,4,5,5-tetramethyl-1,3,2-dioxaborolan-2-yl)phenyl]-1,4-diazabicyclo[3.2.1]octane

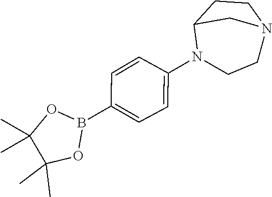 [SMILES: CC1(C)OB(c2ccc(N3CCN4CCC3C4)cc2)OC1(C)C]

By analogy with Example 5 Step 1, 4-(4-bromophenyl)-1,4-diazabicyclo[3.2.1]octane (596 mg, 2.23 mmol, Eq: 1.0) was reacted with bis(pinacolato)diboron in the presence of KOAc and Pd(dppf)Cl₂·CH₂Cl₂ at 90° C. for 3 h. Purification by flash chromatography (silica gel, 24 g, 0% to 10% MeOH in (DCM:MeOH:NH₄OH 9:1:0.05)) gave the title compound as a dark brown solid (453 mg, 80% purity, 51% yield). m/z 315.0 [M+H]⁺, ESI pos.

### Step 3: ethyl 2-[6-[4-(1,4-diazabicyclo[3.2.1]octan-4-yl)phenyl]-4-(difluoromethyl)-7-methyl-indazol-2-yl]-2-[(6R)-6-fluoro-6,7-dihydro-5H-pyrrolo[1,2-c]imidazol-1-yl]acetate

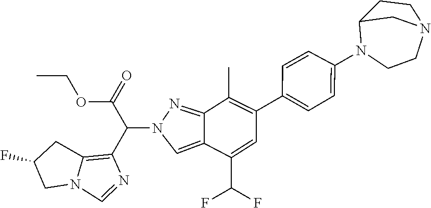 [SMILES: CCOC(=O)C(c1ncn2c1C[C@@H](F)C2)n1cc2c(C(F)F)cc(-c3ccc(N4CCN5CCC4C5)cc3)c(C)c2n1]

By analogy with Example 2 Step 7, Intermediate 1 (100 mg, 212 μmol, Eq: 1.0) and 4-[4-(4,4,5,5-tetramethyl-1,3,2-dioxaborolan-2-yl)phenyl]-1,4-diazabicyclo[3.2.1]octane (100 mg, 318 μmol, Eq: 1.5) were reacted in the presence of Cs₂CO₃ and Pd(dppf)Cl₂·CH₂Cl₂ at 65° C. for 1.5 h. Purification by flash chromatography (silica gel, 12 g, 0% to 10% MeOH in (DCM:MeOH:NH₄OH 9:1:0.05)) gave the title compound as a yellow solid (90 mg, 82% purity, 73% yield). m/z 579.3 [M+H]⁺, ESI pos.

### Step 4: 2-[6-[4-(1,4-diazabicyclo[3.2.1]octan-4-yl)phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

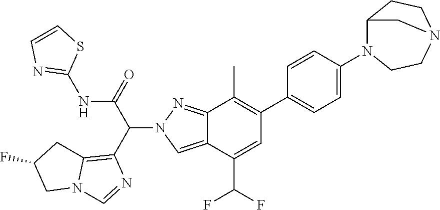 [SMILES: Cc1c(-c2ccc(N3CCN4CCC3C4)cc2)cc(C(F)F)c2cn(C(C(=O)Nc3nccs3)c3ncn4c3C[C@@H](F)C4)nc12]

By analogy with Example 2 Step 8, ethyl 2-[6-[4-(1,4-diazabicyclo[3.2.1]octan-4-yl)phenyl]-4-(difluoromethyl)-7-methyl-indazol-2-yl]-2-[(6R)-6-fluoro-6,7-dihydro-5H-pyrrolo[1,2-c]imidazol-1-yl]acetate (0.09 g, 124 μmol, Eq: 1.0) was initially treated with LiOH, and the resulting salt was reacted with 2-aminothiazole in the presence of HATU and DIPEA. Purification by flash chromatography (silica gel, 12 g, 0% to 100% (DCM:MeOH:NH₄OH 9:1:0.05) in DCM) gave the title compound as a yellow solid (43 mg, 92% purity, 50% yield). m/z 633.3 [M+H]⁺, ESI pos.

### Example 14

### 2-[4-(Difluoromethyl)-6-[4-[2-[4-(1-hydroxyethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

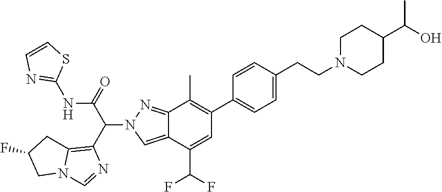 [SMILES: Cc1c(-c2ccc(CCN3CCC(C(C)O)CC3)cc2)cc(C(F)F)c2cn(C(C(=O)Nc3nccs3)c3ncn4c3C[C@@H](F)C4)nc12]

### Step 1: 1-[1-[2-(4-bromophenyl)ethyl]-4-piperidyl]ethanol

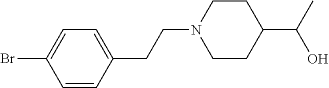 [SMILES: CC(O)C1CCN(CCc2ccc(Br)cc2)CC1]

To a solution of 2-(4-bromophenyl)acetaldehyde (CAS 27200-79-9, 1.00 g, 5.02 mmol, Eq: 1.0) and 1-(4-piperidyl)ethanol (CAS 6457-48-3, 844 mg, 6.53 mmol, Eq: 1.3) in DCM (10 mL) was added sodium triacetoxyborohydride (3.19 g, 15.1 mmol, Eq: 3.0). The reaction mixture was stirred for 16 h at rt. The reaction mixture was quenched with sat. aq. Na₂CO₃ and extracted twice with EtOAc. The organic layers were washed with water and brine. The combined organic layers were dried over Na₂SO₄ and concentrated in vacuo. The crude material was purified by flash chromatography (silica gel, 80 g, 0% to 20% MeOH in DCM) to give the title compound as a colorless oil (1.15 g, 23% yield). m/z 314.0 [M+H]⁺, ESI pos.

### Step 2: 1-[1-[2-[4-(4,4,5,5-tetramethyl-1,3,2-dioxaborolan-2-yl)phenyl]ethyl]-4-piperidyl]ethanol

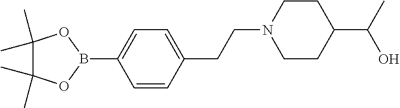 [SMILES: CC(O)C1CCN(CCc2ccc(B3OC(C)(C)C(C)(C)O3)cc2)CC1]

By analogy with Example 5 Step 1, 1-[1-[2-(4-bromophenyl)ethyl]-4-piperidyl]ethanol (1.15 g, 3.68 mmol, Eq: 1.0) was reacted with bis(pinacolato)diboron in the presence of KOAc and Pd(dppf)Cl₂·CH₂Cl₂ for 3 h at 90° C. Purification by flash chromatography (silica gel, 24 g, 0% to 100% (DCM:MeOH:NH₄OH 9:1:0.05) in DCM) gave the title compound as a dark brown oil (1.35 g, 60% purity, 61% yield). m/z 360.3 [M+H]⁺, ESI pos.

### Step 3: ethyl 2-[4-(difluoromethyl)-6-[4-[2-[4-(1-hydroxyethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]acetate

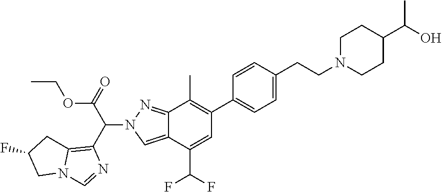 [SMILES: CCOC(=O)C(c1ncn2c1C[C@@H](F)C2)n1cc2c(C(F)F)cc(-c3ccc(CCN4CCC(C(C)O)CC4)cc3)c(C)c2n1]

By analogy with Example 2 Step 7, Intermediate 1 (100 mg, 0.212 mmol, Eq: 1.0) and 1-[1-[2-[4-(4,4,5,5-tetramethyl-1,3,2-dioxaborolan-2-yl)phenyl]ethyl]-4-piperidyl]ethanol (191 mg, 0.318 mmol, Eq: 1.5, 60% purity) were reacted in the presence of Cs₂CO₃ and Pd(dppf)Cl₂·CH₂Cl₂ for 2 h at 65° C. Purification by flash chromatography (silica gel, 12 g, 0% to 100% (DCM:MeOH:NH₄OH 9:1:0.05) in DCM) gave the title compound as a light yellow solid (43 mg, 33% yield). m/z 624.3 [M+H]⁺, ESI pos.

### Step 4: 2-[4-(difluoromethyl)-6-[4-[2-[4-(1-hydroxyethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

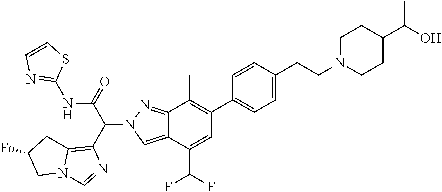 [SMILES: Cc1c(-c2ccc(CCN3CCC(C(C)O)CC3)cc2)cc(C(F)F)c2cn(C(C(=O)Nc3nccs3)c3ncn4c3C[C@@H](F)C4)nc12]

By analogy with Example 8 Step 2, ethyl 2-[4-(difluoromethyl)-6-[4-[2-[4-(1-hydroxyethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]acetate (43 mg, 0.069 mmol, Eq: 1.0) was initially treated with LiOH then with aq. HCl, and the resultant carboxylic acid was reacted with 2-aminothiazole in the presence of HATU and DIPEA. Purification by flash chromatography (silica gel, 12 g, 0% to 10% MeOH in (DCM:MeOH:NH₄OH 9:1:0.05)) gave the title compound as a yellow solid (14 mg, 30% yield). m/z 678.4 [M+H]⁺, ESI pos.

### Example 15

### 2-[4-(Difluoromethyl)-6-[4-[3-[[4-(hydroxymethyl)-1-piperidyl]methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

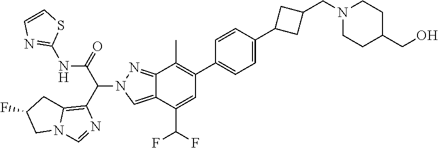 [SMILES: Cc1c(-c2ccc(C3CC(CN4CCC(CO)CC4)C3)cc2)cc(C(F)F)c2cn(C(C(=O)Nc3nccs3)c3ncn4c3C[C@@H](F)C4)nc12]

### Step 1: [1-[[3-(4-bromophenyl)cyclobutyl]methyl]-4-piperidyl]methanol

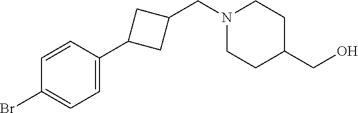 [SMILES: OCC1CCN(CC2CC(c3ccc(Br)cc3)C2)CC1]

To a solution of [3-(4-bromophenyl)cyclobutyl]methanol (CAS 1782303-81-4, 0.74 g, 3.07 mmol, Eq: 1) in DCM (14 mL) and NEt₃ (621 mg, 0.856 mL, 6.14 mmol, Eq: 2) was added methanesulfonyl chloride (422 mg, 0.287 mL, 3.68 mmol, Eq: 1.2) at 0° C. The reaction mixture was stirred for 2 h at 0° C. to 20° C. The reaction mixture was extracted with sat. aq. NaHCO₃ and two times with DCM. The organic layers were washed with water, dried over Na₂SO₄ and concentrated in vacuo to give presumed [3-(4-bromophenyl)cyclobutyl]methyl methanesulfonate as a light brown oil (1.1 g, 85% purity, 95% yield) which was used in the next step without further purification.

To a solution of presumed [3-(4-bromophenyl)cyclobutyl]methyl methanesulfonate (550 mg, 1.72 mmol, Eq: 1.0) in acetonitrile (2 mL) were added 4-piperidyl MeOH (CAS 6457-49-4, 298 mg, 2.58 mmol, Eq: 1.5) and NEt₃ (523 mg, 0.72 mL, 5.17 mmol, Eq: 3). The reaction mixture was stirred for 1 h at 20° C. and for 16 h at 60° C. The reaction mixture was concentrated in vacuo. The residue was diluted with sat. aq. NaHCO₃ and extracted two times with EtOAc. The organic layers were washed with water and brine. The combined organic layers were dried over Na₂SO₄ and concentrated in vacuo. The crude material was purified by flash chromatography (silica gel, 12 g, 0% to 20% MeOH in DCM) to give the title compound as a light yellow oil (270 mg, 46% yield). m/z 338.1, 339.9 [M+H]⁺, ESI pos, Br isotopes.

### Step 2: [1-[[3-[4-(4,4,5,5-tetramethyl-1,3,2-dioxaborolan-2-yl)phenyl]cyclobutyl]methyl]-4-piperidyl]methanol

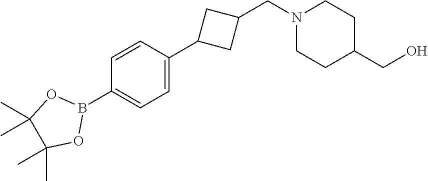 [SMILES: CC1(C)OB(c2ccc(C3CC(CN4CCC(CO)CC4)C3)cc2)OC1(C)C]

By analogy with Example 5 Step 1, [1-[[3-(4-bromophenyl)cyclobutyl]methyl]-4-piperidyl]methanol (270 mg, 0.798 mmol, Eq: 1.0) was reacted with bis(pinacolato)diboron in the presence of KOAc and Pd(dppf)Cl₂·CH₂Cl₂ for 3 h at 90° C. Purification by flash chromatography (silica gel, 24 g, 0% to 20% MeOH in DCM) gave the title compound as a brown solid (138 mg, 70% purity, 31% yield). m/z 386.2 [M+H]⁺, ESI pos.

### Step 3: ethyl 2-[4-(difluoromethyl)-6-[4-[3-[[4-(hydroxymethyl)-1-piperidyl]methyl]cyclobutyl]phenyl]-7-methyl-indazol-2-yl]-2-[(6R)-6-fluoro-6,7-dihydro-5H-pyrrolo[1,2-c]imidazol-1-yl]acetate

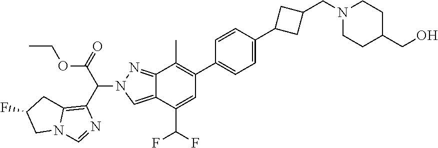 [SMILES: CCOC(=O)C(c1ncn2c1C[C@@H](F)C2)n1cc2c(C(F)F)cc(-c3ccc(C4CC(CN5CCC(CO)CC5)C4)cc3)c(C)c2n1]

By analogy with Example 2 Step 7, Intermediate 1 (100 mg, 0.212 mmol, Eq: 1.0) and [1-[[3-[4 (4,4,5,5-tetramethyl-1,3,2-dioxaborolan-2-yl)phenyl]cyclobutyl]methyl]-4-piperidyl]methanol (138 mg, 0.358 mmol, Eq: 1.69) were reacted in the presence of Cs₂CO₃ and Pd(dppf)Cl₂·CH₂Cl₂ for 1.5 h at 65° C. Purification by flash chromatography (silica gel, 12 g, 0% to 100% (DCM:MeOH:NH₄OH 9:1:0.05) in DCM) gave the title compound as a light yellow solid (79 mg, 88% purity, 50% yield). m/z 650.5 [M+H]⁺, ESI pos.

### Step 4: 2-[4-(difluoromethyl)-6-[4-[3-[[4-(hydroxymethyl)-1-piperidyl]methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

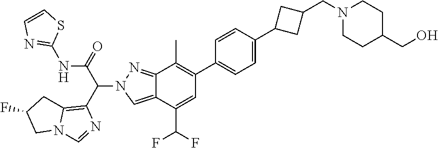 [SMILES: Cc1c(-c2ccc(C3CC(CN4CCC(CO)CC4)C3)cc2)cc(C(F)F)c2cn(C(C(=O)Nc3nccs3)c3ncn4c3C[C@@H](F)C4)nc12]

By analogy with Example 8 Step 2, ethyl 2-[4-(difluoromethyl)-6-[4-[3-[[4-(hydroxymethyl)-1-piperidyl]methyl]cyclobutyl]phenyl]-7-methyl-indazol-2-yl]-2-[(6R)-6-fluoro-6,7-dihydro-5H-pyrrolo[1,2-c]imidazol-1-yl]acetate (79 mg, 0.121 mmol, Eq: 1.0) was initially treated with LiOH then with aq. HCl, and the resultant carboxylic acid was reacted with 2-aminothiazole in the presence of HATU and DIPEA. Purification by flash chromatography (silica gel, 12 g, 0% to 10% MeOH in EtOAc) gave the title compound as a light brown solid (39 mg, 93% purity, 42% yield). m/z 704.5 [M+H]⁺, ESI pos.

### Example 16

### 2-[4-(Difluoromethyl)-7-methyl-6-[4-(4-methylsulfonyl-1-piperidyl)phenyl]indazol-2-yl]-2-[(6R)-6-fluoro-6,7-dihydro-5H-pyrrolo[1,2-c]imidazol-1-yl]-N-thiazol-2-yl-acetamide

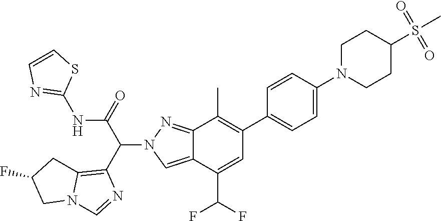 [SMILES: Cc1c(-c2ccc(N3CCC(S(C)(=O)=O)CC3)cc2)cc(C(F)F)c2cn(C(C(=O)Nc3nccs3)c3ncn4c3C[C@@H](F)C4)nc12]

### Step 1: 1-(4-bromophenyl)-4-methylsulfonyl-piperidine

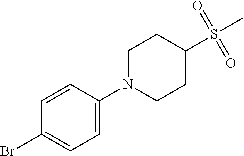 [SMILES: CS(=O)(=O)C1CCN(c2ccc(Br)cc2)CC1]

By analogy with Example 13 Step 1, 1-bromo-4-iodo-benzene (953 mg, 3.37 mmol, Eq: 1.1) and 4-methylsulfonylpiperidine (CAS 290328-55-1, 500 mg, 3.06 mmol, Eq: 1.0) were reacted in the presence of NaO^{t}Bu, xantphos and Pd₂(dba)₃·CHCl₃ for 16 h at 80° C. Purification by flash chromatography (silica gel, 40 g, 0% to 70% EtOAc in heptane) gave the title compound as a brown solid (522 mg, 51% yield). m/z 318.0, 320.0 [M+H]⁺, ESI pos, Br isotopes.

### Step 2: 4-methylsulfonyl-1-[4-(4,4,5,5-tetramethyl-1,3,2-dioxaborolan-2-yl)phenyl]piperidine

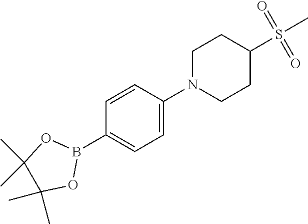

By analogy with Example 5 Step 1, 1-(4-bromophenyl)-4-methylsulfonyl-piperidine (350 mg, 1.04 mmol, Eq: 1) was reacted with bis(pinacolato)diboron in the presence of KOAc and Pd(dppf)Cl₂·CH₂Cl₂ for 5 h at 90° C. and for 16 h at rt. Purification by flash chromatography (silica gel, 24 g, 0% to 70% EtOAc in heptane) gave the title compound as a light brown oil (160 mg, 90% purity, 38% yield). m/z 366.2 [M+H]⁺, ESI pos.

### Step 3: Ethyl 2-[4-(difluoromethyl)-7-methyl-6-[4-(4-methylsulfonyl-1-piperidyl)phenyl]indazol-2-yl]-2-[(6R)-6-fluoro-6,7-dihydro-5H-pyrrolo[1,2-c]imidazol-1-yl]acetate

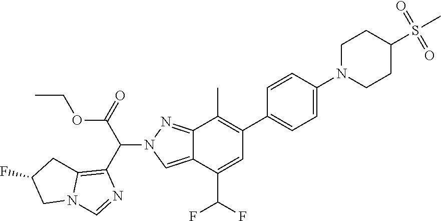 [SMILES: CCOC(=O)C(c1ncn2c1C[C@@H](F)C2)n1cc2c(C(F)F)cc(-c3ccc(N4CCC(S(C)(=O)=O)CC4)cc3)c(C)c2n1]

By analogy with Example 2 Step 7, Intermediate 1 (120 mg, 0.255 mmol, Eq: 1.0) and 4-methylsulfonyl-1-[4-(4,4,5,5-tetramethyl-1,3,2-dioxaborolan-2-yl)phenyl]piperidine (155 mg, 0.3 82 mmol, Eq: 1.5, 90% purity), were reacted in the presence of Cs₂CO₃ and Pd(dppf)Cl₂·CH₂Cl₂ for 2.25 h at 65° C., followed by the addition of further Pd(dppf)Cl₂·CH₂Cl₂ and reaction for 45 min at 65° C. Purification by flash chromatography (silica gel, 12 g, 0% to 5% MeOH in DCM) gave the title compound as a brown oil (66 mg, 90% purity, 37% yield). m/z 630.3 [M+H]⁺, ESI pos.

### Step 4: 2-[4-(difluoromethyl)-7-methyl-6-[4-(4-methylsulfonyl-1-piperidyl)phenyl]indazol-2-yl]-2-[(6R)-6-fluoro-6,7-dihydro-5H-pyrrolo[1,2-c]imidazol-1-yl]-N-thiazol-2-yl-acetamide

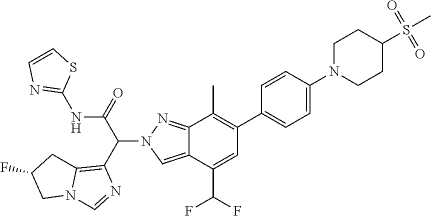 [SMILES: Cc1c(-c2ccc(N3CCC(S(C)(=O)=O)CC3)cc2)cc(C(F)F)c2cn(C(C(=O)Nc3nccs3)c3ncn4c3C[C@@H](F)C4)nc12]

By analogy with Example 8 Step 2, ethyl 2-[4-(difluoromethyl)-7-methyl-6-[4-(4-methylsulfonyl-1-piperidyl)phenyl]indazol-2-yl]-2-[(6R)-6-fluoro-6,7-dihydro-5H-pyrrolo[1,2-c]imidazol-1-yl]acetate (63 mg, 0.090 mmol, Eq: 1.0, 90% purity) was initially treated with LiOH then with aq. HCl, and the resulting carboxylic acid was reacted with 2-aminothiazole in the presence of HATU and DIPEA. Purification by flash chromatography (silica gel, 12 g, 0% to 60% (DCM:MeOH:NH₄OH 9:1:0.05) in DCM) followed by repurificaiton of product-containing fractions by flash chromatography (silica gel, 12 g, 0% to 10% MeOH in DCM) gave the title compound as a light brown foam (25 mg, 39% yield). m/z 684.3 [M+H]⁺, ESI pos.

### Example 17

### 2-[4-(Difluoromethyl)-6-[4-[3-[(4-hydroxy-1-piperidyl)methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

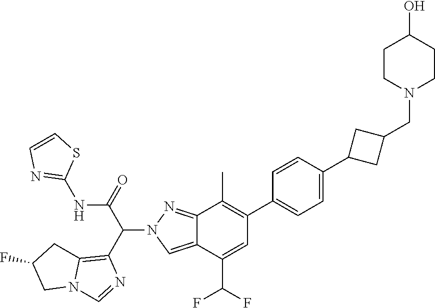 [SMILES: Cc1c(-c2ccc(C3CC(CN4CCC(O)CC4)C3)cc2)cc(C(F)F)c2cn(C(C(=O)Nc3nccs3)c3ncn4c3C[C@@H](F)C4)nc12]

### Step 1: [3-(4-bromophenyl)cyclobutyl]methanol

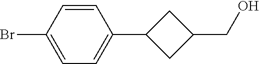 [SMILES: OCC1CC(c2ccc(Br)cc2)C1]

3-(4-Bromophenyl)cyclobutane-1-carboxylic acid (CAS 149506-16-1, 1 g, 3.92 mmol, Eq: 1.0) was suspended in THE (10 mL) and borane-tetrahydrofuran complex (1 M, 7.84 mL, 7.84 mmol, Eq: 2.0) was added at 0° C. The mixture was stirred for 30 min at 5° C., then the mixture was warmed up to 20° C. and stirred for 1 h, after which time further borane-tetrahydrofuran complex (1 M, 7.84 mL, 7.84 mmol, Eq: 2.0) was added and the mixture was stirred overnight. NaOH (2 N aq.) was added to the reaction mixture until pH=10. The reaction mixture was diluted with H₂O and extracted two times with EtOAc. The organic layers were washed with water and brine, dried over Na₂SO₄, filtered and evaporated to dryness. The crude material was purified by flash chromatography (silica gel, 40 g, 0% to 100% EtOAc in heptane) to give the title compound as a colorless oil (740 mg, 85% purity, 66% yield). m/z 224.9 [M−H₂O+H]⁺, ESI pos.

### Step 2: 1-[[3-(4-bromophenyl)cyclobutyl]methyl]piperidin-4-ol

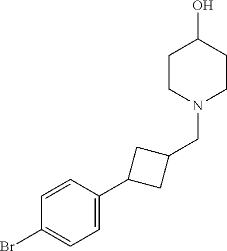 [SMILES: OC1CCN(CC2CC(c3ccc(Br)cc3)C2)CC1]

[3-(4-Bromophenyl)cyclobutyl]methanol (0.74 g, 3.07 mmol, Eq: 1.0) was dissolved in DCM (14 mL), then Et₃N (621 mg, 856 μL, 6.14 mmol, Eq: 2.0) and methanesulfonyl chloride (422 mg, 287 μL, 3.68 mmol, Eq: 1.2) were added at rt. The mixture was stirred for 2 h at 0-20° C. The reaction mixture was diluted with sat. aq. NaHCO₃ and extracted two times with DCM. The organic layers were washed with water, dried over Na₂SO₄, filtered and evaporated to dryness to give presumed [1-[[3-(4-bromophenyl)cyclobutyl]methyl]-4-piperidyl]methanesulfonate as a light brown oil (1.1 g) which was used without further purification.

550 mg (1.72 mmol, Eq: 1.0) of the crude material was dissolved in acetonitrile (2 mL), then piperidin-4-ol (261 mg, 2.58 mmol, Eq: 1.5) and Et₃N (523 mg, 720 μL, 5.17 mmol, Eq: 3.0) were added at rt. The mixture was stirred for 16 h at 60° C. The reaction mixture was evaporated and diluted with sat. aq. NaHCO₃ and extracted two times with EtOAc. The organic layers were extracted with water and brine, dried over Na₂SO₄, filtered and evaporated to dryness. The crude material was purified by flash chromatography (silica gel, 12 g, 0% to 20% MeOH in DCM) to give the title compound as a colorless oil (323 mg, 81% purity, 43% yield). m/z 325.9 [M+H]⁺, ESI pos.

### Step 3: 1-[[3-[4-(4,4,5,5-tetramethyl-1,3,2-dioxaborolan-2-yl)phenyl]cyclobutyl]methyl]piperidin-4-ol

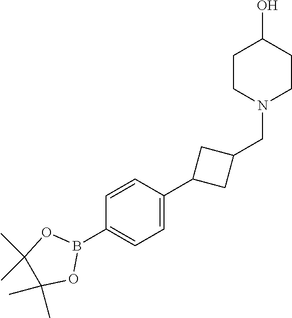 [SMILES: CC1(C)OB(c2ccc(C3CC(CN4CCC(O)CC4)C3)cc2)OC1(C)C]

By analogy with Example 5 Step 1, 1-[[3-(4-bromophenyl)cyclobutyl]methyl]piperidin-4-ol (0.245 g, 756 μmol, Eq: 1.0) was reacted with bis(pinacolato)diboron in the presence of KOAc and Pd(dppf)Cl₂·CH₂Cl₂ at 90° C. for 2 h. Purification by flash chromatography (silica gel, 12 g, 0% to 100% (DCM:MeOH:NH₄OH 9:1:0.05) in DCM) gave the title compound as a light brown solid (260 mg, 86% purity, 92% yield). m/z 372.1 [M+H]⁺, ESI pos.

### Step 4: ethyl 2-[4-(difluoromethyl)-6-[4-[3-[(4-hydroxy-1-piperidyl)methyl]cyclobutyl]phenyl]-7-methyl-indazol-2-yl]-2-[(6R)-6-fluoro-6,7-dihydro-5H-pyrrolo[1,2-c]imidazol-1-yl]acetate

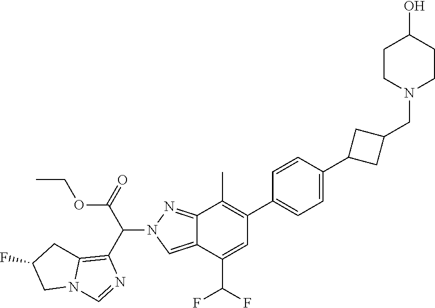 [SMILES: CCOC(=O)C(c1ncn2c1C[C@@H](F)C2)n1cc2c(C(F)F)cc(-c3ccc(C4CC(CN5CCC(O)CC5)C4)cc3)c(C)c2n1]

By analogy with Example 2 Step 7, Intermediate 2 (130 mg, 276 μmol, Eq: 1.0) and 1-[[3-[4-(4,4,5,5-tetramethyl-1,3,2-dioxaborolan-2-yl)phenyl]cyclobutyl]methyl]piperidin-4-ol (260 mg, 420 μmol, Eq: 1.52), were reacted in the presence of Cs₂CO₃ and Pd(dppf)Cl₂·CH₂Cl₂ at 70° C. for 2.5 h. Purification by flash chromatography (silica gel, 25 g, 0% to 100% (DCM:MeOH:NH₄OH 9:1:0.05) in DCM) gave the title compound as a light brown solid (60 mg, 72% purity, 34% yield). m/z 636.4 [M+H]⁺, ESI pos.

### Step 5: 2-[4-(difluoromethyl)-6-[4-[3-[(4-hydroxy-1-piperidyl)methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

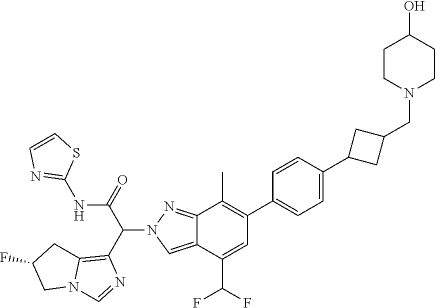 [SMILES: Cc1c(-c2ccc(C3CC(CN4CCC(O)CC4)C3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[3-[(4-hydroxy-1-piperidyl)methyl]cyclobutyl]phenyl]-7-methyl-indazol-2-yl]-2-[(6R)-6-fluoro-6,7-dihydro-5H-pyrrolo[1,2-c]imidazol-1-yl]acetate (0.06 g, 94.4 μcool, Eq: 1.0) was initially treated with LiOH, and the resulting salt was reacted with 2-aminothiazole in the presence of HATU and DIPEA. Purification by flash chromatography (silica gel, 12 g, 0% to 100% (DCM:MeOH:NH₄OH 9:1:0.05) in DCM) gave the title compound as a light yellow foam (25 mg, 90% purity, 34% yield). m/z 690.4 [M+H]⁺, ESI pos.

### Example 18

### 2-[4-(Difluoromethyl)-7-methyl-6-[4-(2-morpholinoethoxy)phenyl]indazol-2-yl]-2-[(6R)-6-fluoro-6,7-dihydro-5H-pyrrolo[1,2-c]imidazol-1-yl]-N-thiazol-2-yl-acetamide

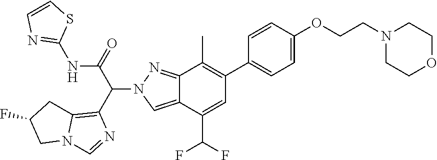 [SMILES: Cc1c(-c2ccc(OCCN3CCOCC3)cc2)cc(C(F)F)c2cn(C(C(=O)Nc3nccs3)c3ncn4c3C[C@@H](F)C4)nc12]

### Step 1: Ethyl 2-[4-(difluoromethyl)-7-methyl-6-[4-(2-morpholinoethoxy)phenyl]indazol-2-yl]-2-[(6R)-6-fluoro-6,7-dihydro-5H-pyrrolo[1,2-c]imidazol-1-yl]acetate

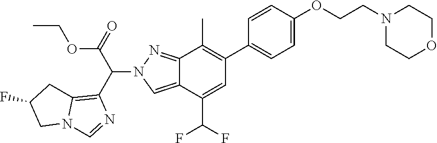 [SMILES: CCOC(=O)C(c1ncn2c1C[C@@H](F)C2)n1cc2c(C(F)F)cc(-c3ccc(OCCN4CCOCC4)cc3)c(C)c2n1]

By analogy with Example 2 Step 7, Intermediate 1 (130 mg, 276 μmol, Eq: 1.0) and 4-[2-[4-(4,4,5,5-tetramethyl-1,3,2-dioxaborolan-2-yl)phenoxy]ethyl]morpholine (CAS 690636-28-3, 110 mg, 331 μmol, Eq: 1.2) were reacted in the presence of K₂CO₃ and Pd(dppf)Cl₂·CH₂Cl₂ for 3 hat 70° C. Purification by flash chromatography (silica gel, 12 g, 0% to 10% MeOH in EtOAc) gave the title compound as a light brown foam (53 mg, 90% purity, 29% yield). m/z 598.4 [M+H]⁺, ESI pos.

### Step 2: 2-[4-(difluoromethyl)-7-methyl-6-[4-(2-morpholinoethoxy)phenyl]indazol-2-yl]-2-[(6R)-6-fluoro-6,7-dihydro-5H-pyrrolo[1,2-c]imidazol-1-yl]-N-thiazol-2-yl-acetamide

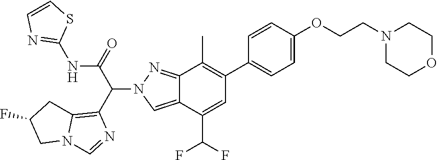 [SMILES: Cc1c(-c2ccc(OCCN3CCOCC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-7-methyl-6-[4-(2-morpholinoethoxy)phenyl]indazol-2-yl]-2-[(6R)-6-fluoro-6,7-dihydro-5H-pyrrolo[1,2-c]imidazol-1-yl]acetate (52 mg, 87 μmol, Eq: 1.0) was initially treated with LiOH, and the resulting salt was reacted with 2-aminothiazole in the presence of HATU and DIPEA. Purification by flash chromatography (silica gel, 12 g, 0% to 20% MeOH in DCM) gave the title compound as an off-white solid (35 mg, 58% yield). m/z 652.3 [M+H]⁺, ESI pos.

### Example 19

### 2-(4-(Difluoromethyl)-6-(4-(3-((4-(hydroxymethyl)piperidin-1-yl)methyl)azetidin-1-yl)phenyl)-7-methyl-211-indazol-2-yl)-2-((R)-6-fluoro-6,7-dihydro-5H-pyrrolo[1,2-c]imidazol-1-yl)-N-(thiazol-2-yl)acetamide

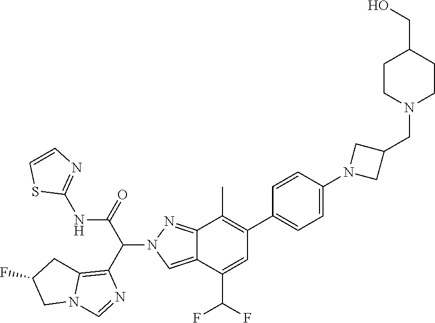 [SMILES: Cc1c(-c2ccc(N3CC(CN4CCC(CO)CC4)C3)cc2)cc(C(F)F)c2cn(C(C(=O)Nc3nccs3)c3ncn4c3C[C@@H](F)C4)nc12]

### Step 1: (1-(4-bromophenyl)azetidin-3-yl)methanol

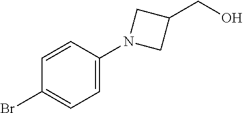 [SMILES: OCC1CN(c2ccc(Br)cc2)C1]

To a solution of 1-bromo-4-iodobenzene (6 g, 21.2 mmol, Eq: 1.0) in DMF (11.3 mL) was added azetidin-3-ylmethanol hydrochloride (CAS 928038-44-2, 2.79 g, 22.5 mmol, Eq: 1.06), copper (I) iodide (1.62 g, 8.48 mmol, Eq: 0.40), K₃PO₄ (18 g, 84.8 mmol, Eq: 4.0) and (S)-(−)-1,1′-bi-2-naphthol (1.21 g, 4.24 mmol, Eq: 0.20), with nitrogen bubbling through the solution. The mixture was stirred for 16 h at 100° C. under nitrogen. After cooling, water and EtOAc were added. After separation, the aqueous layer was extracted again with EtOAc. The combined organic layers were washed with brine, dried over Na₂SO₄ and evaporated. The crude material was purified by flash chromatography (silica gel, 330 g, 0% to 50% EtOAc in heptane) to give the title compound as a light brown solid (663 mg, 12% yield). m/z 242.1 [M+H]⁺, ESI pos, Br isotopes

### Step 2: (1-(4-bromophenyl)azetidin-3-yl)methyl Methanesulfonate

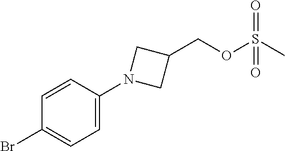 [SMILES: CS(=O)(=O)OCC1CN(c2ccc(Br)cc2)C1]

To a stirred solution of (1-(4-bromophenyl)azetidin-3-yl)methanol (400 mg, 1.65 mmol, Eq: 1.0) and NEt₃ (284 mg, 391 μL, 2.81 mmol, Eq: 1.7) in THE (27.5 mL) was added methanesulfonyl chloride (227 mg, 154 μL, 1.98 mmol, Eq: 1.2) at 0° C. and the mixture was allowed to warm up to rt and stirred for 3 h. The mixture was diluted with water and extracted twice with EtOAc. The combined organic phases were dried over Na₂SO₄ and concentrated to give the title compound as a light brown solid (502 mg, 95% yield) which was used without further purification in the next step. m/z 320.0 [M+H]⁺, ESI pos, Br isotopes.

### Step 3: 4-(((tert-butyldimethylsilyl)oxy)methyl)piperidine

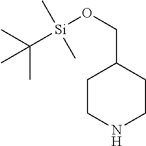

To a solution of piperidin-4-ylmethanol (1.0 g, 8.68 mmol, Eq: 1.0) in DMF (5 mL) was added TBDMS-Cl (1.31 g, 8.68 mmol, Eq: 1.0) and 1H-imidazole (1.18 g, 17.4 mmol, Eq: 2.0). The reaction mixture was stirred overnight at rt then diluted with water and extracted with TBME twice. To the stirred combined organic layers was added water followed by AcOH (2.61 g, 2.49 mL, 43.4 mmol, Eq: 5.0). After stirring for 5 min, the two layers were separated. The aqueous phase was neutralized by addition of sat. aq. NaHCO₃ to pH 8 and extracted three times with TBME. The combined organic layers were dried over Na₂SO₄ and evaporated to give the title compound as a pale yellow liquid (0.69 g, 35% yield) which was used without further purification in the next step. ¹H NMR (300 MHz, chloroform-d) δ ppm 0.04 (s, 6H) 0.89 (s, 9H) 1.03-1.21 (m, 2H) 1.51-1.65 (m, 1H) 1.65-1.75 (m, 2H) 2.04 (br s, 1H) 2.52-2.68 (m, 2H) 3.04-3.15 (m, 2H) 3.38-3.48 (m, 2H)

### Step 4: 1-((1-(4-bromophenyl)azetidin-3-yl)methyl)-4-(((tert-butyldimethylsilyl)oxy)methyl)piperidine

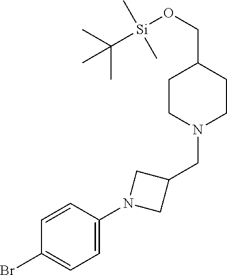 [SMILES: CC(C)(C)[Si](C)(C)OCC1CCN(CC2CN(c3ccc(Br)cc3)C2)CC1]

To a solution of (1-(4-bromophenyl)azetidin-3-yl)methyl methanesulfonate (110 mg, 343 μmol, Eq: 1.0) in DMF (0.5 mL) were added DIPEA (133 mg, 180 μL, 1.03 mmol, Eq: 3.0) and 4-(((tert-butyldimethylsilyl)oxy)methyl)piperidine (94.4 mg, 412 μcool, Eq: 1.2) at rt. The reaction mixture was stirred for 20 hours at 80° C. The reaction mixture was diluted with EtOAc and washed with sat. NaHCO₃. The aqueous layer was extracted a second time and the combined organic layers were dried over Na₂SO₄, filtered and evaporated. The crude material was purified by flash chromatography (silica gel, 4 g, 0% to 10% MeOH in DCM) to give the title compound as an off white solid (80 mg, 51% yield). m/z 455.2 [M+H]⁺, ESI pos.

### Step 5: Ethyl 2-(6-(4-(3-((4-(((tert-butyldimethylsilyl)oxy)methyl)piperidin-1-yl)methyl)azetidin-1-yl)phenyl)-4-(difluoromethyl)-7-methyl-2H-indazol-2-yl)-2-((R)-6-fluoro-6,7-dihydro-5H-pyrrolo[1,2-c]imidazol-1-yl)acetate

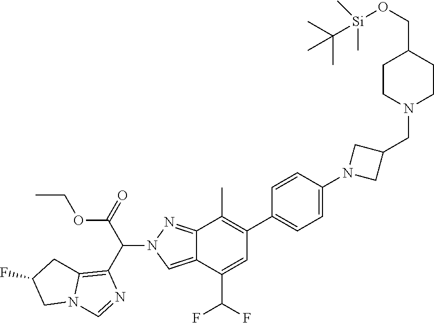 [SMILES: CCOC(=O)C(c1ncn2c1C[C@@H](F)C2)n1cc2c(C(F)F)cc(-c3ccc(N4CC(CN5CCC(CO[Si](C)(C)C(C)(C)C)CC5)C4)cc3)c(C)c2n1]

In a vial, to a solution of intermediate 3 [ethyl 2-(4-(difluoromethyl)-7-methyl-6-(4,4,5,5-tetramethyl-1,3,2-dioxaborolan-2-yl)-2H-indazol-2-yl)-2-((R)-6-fluoro-6,7-dihydro-5H-pyrrolo[1,2-c]imidazol-1-yl)acetate](80 mg, 154 μmol, Eq: 1.0) in THE (1 mL) and water (167 μL) were added while bubbling argon through the solution 1-((1-(4-bromophenyl)azetidin-3-yl)methyl)-4-(((tert-butyldimethylsilyl)oxy)methyl)piperidine (70 mg, 154 μmol, Eq: 1.0), Cs₂CO₃ (151 mg, 463 μmol, Eq: 3.0), and Pd(dppf)Cl₂·CH₂Cl₂ (22.6 mg, 30.9 μmol, Eq: 0.20). The vial was closed and the reaction mixture was stirred at 70° C. for 2 h. The reaction mixture was partitioned between water and EtOAc. The aqueous layer was extracted 2 times with EtOAc, then the combined organic phases were washed with water and brine, and dried over Na₂SO₄. The solvent was evaporated in vacuo, and the crude material was purified by flash chromatography (silica gel, 10 g, 0% to 100% MeOH in DCM) to give the title compound as an off-white foam (29 mg, 25% yield). m/z 765.5 [M+H]⁺, ESI pos.

### Step 6: 2-(6-(4-(3-((4-(((tert-butyldimethylsilyl)oxy)methyl)piperidin-1-yl)methyl)azetidin-1-yl)phenyl)-4-(difluoromethyl)-7-methyl-2H-indazol-2-yl)-2-((R)-6-fluoro-6,7-dihydro-5H-pyrrolo[1,2-c]imidazol-1-yl)-N-(thiazol-2-yl)acetamide

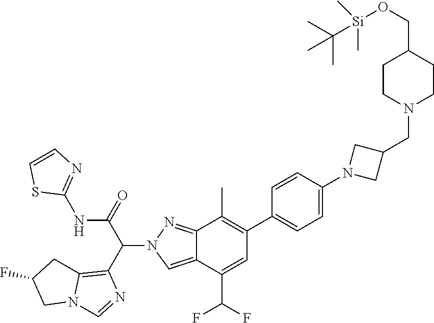 [SMILES: Cc1c(-c2ccc(N3CC(CN4CCC(CO[Si](C)(C)C(C)(C)C)CC4)C3)cc2)cc(C(F)F)c2cn(C(C(=O)Nc3nccs3)c3ncn4c3C[C@@H](F)C4)nc12]

To a solution of ethyl 2-(6-(4-(3-((4-(((tert-butyldimethylsilyl)oxy)methyl)piperidin-1-yl)methyl)azetidin-1-yl)phenyl)-4-(difluoromethyl)-7-methyl-2H-indazol-2-yl)-2-((R)-6-fluoro-6,7-dihydro-5H-pyrrolo[1,2-c]imidazol-1-yl)acetate (30 mg, 39.2 μmol, Eq: 1.0) in EtOH (1 mL) was added LiOH (1 M in water, 39.2 μL, 39.2 μmol, Eq: 1.0). After 1 h, the reaction was concentrated in vacuo and co-evaporated twice with toluene. The residue was dissolved in DMF (1 mL), and DIPEA (15.2 mg, 20.5 μL, 118 μmol, Eq: 3.0), thiazol-2-amine (5.1 mg, 51 μmol, Eq: 1.3) and HATU (19.4 mg, 51 μmol, Eq: 1.3) were added. The reaction was stirred at rt for 2 h, then diluted with water and extracted with EtOAc (2×). The organic layers were dried over Na₂SO₄ and concentrated. The residue was purified by flash chromatography (silica gel, 4 g, 0% to 20% MeOH in DCM) to give the title compound as an off-white foam (10 mg, 90% purity, 27% yield). m/z 819.7 [M+H]⁺, ESI pos.

### Step 7: 2-(4-(difluoromethyl)-6-(4-(3-((4-(hydroxymethyl)piperidin-1-yl)methyl)azetidin-1-yl)phenyl)-7-methyl-2H-indazol-2-yl)-2-((R)-6-fluoro-6,7-dihydro-5H-pyrrolo[1,2-c]imidazol-1-yl)-N-(thiazol-2-yl)acetamide

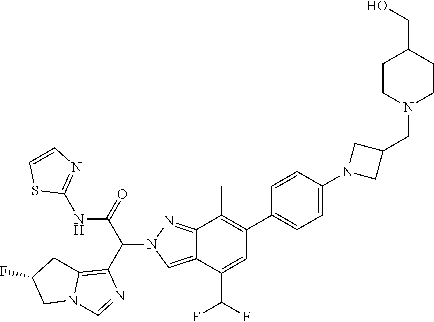 [SMILES: Cc1c(-c2ccc(N3CC(CN4CCC(CO)CC4)C3)cc2)cc(C(F)F)c2cn(C(C(=O)Nc3nccs3)c3ncn4c3C[C@@H](F)C4)nc12]

To a solution of 2-(6-(4-(3-((4-(((tert-butyldimethylsilyl)oxy)methyl)piperidin-1-yl)methyl)azetidin-1-yl)phenyl)-4-(difluoromethyl)-7-methyl-2H-indazol-2-yl)-2-((R)-6-fluoro-6,7-dihydro-5H-pyrrolo[1,2-c]imidazol-1-yl)-N-(thiazol-2-yl)acetamide (10 mg, 12.2 μmol, Eq: 1.0) in THF (0.5 mL) was added tetrabutylammonium fluoride (1M in THF) (24.4 μL, 24.4 μmol, Eq: 2.0). The reaction was stirred for 4 hours, after which time further tetrabutylammonium fluoride (1M in THF) (36.6 μL, 36.6 μmol, Eq: 3.0) was added and the reaction was stirred overnight. The reaction mixture was diluted with 20 mL of EtOAc and washed twice with 20 mL of water and once with 20 mL of brine. The organic layer was dried over Na₂SO₄ and evaporated. The crude material was purified by flash chromatography (Si-amine, 4 g, 0% to 20% MeOH in EtOAc) to give the title compound as a light yellow foam (2.4 mg, 24% yield). m/z 705.4 [M+H]⁺, ESI pos.

### Example 20

### 2-[4-(Difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

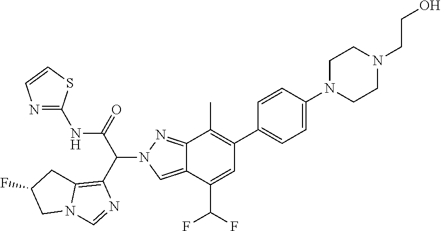 [SMILES: Cc1c(-c2ccc(N3CCN(CCO)CC3)cc2)cc(C(F)F)c2cn(C(C(=O)Nc3nccs3)c3ncn4c3C[C@@H](F)C4)nc12]

### Step 1: Tert-Butyl 4-[4-[4-(difluoromethyl)-2-[2-ethoxy-2-oxo-1-[(6R)-6-fluoro-6,7-dihydro-5H-pyrrolo[1,2-c]imidazol-1-yl]ethyl]-7-methyl-indazol-6-yl]phenyl]piperazine-1-carboxylate

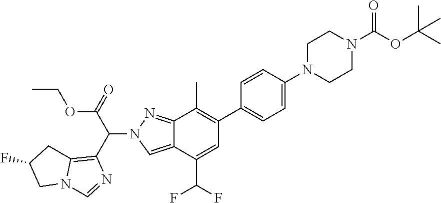 [SMILES: CCOC(=O)C(c1ncn2c1C[C@@H](F)C2)n1cc2c(C(F)F)cc(-c3ccc(N4CCN(C(=O)OC(C)(C)C)CC4)cc3)c(C)c2n1]

A suspension of Intermediate 1 (160 mg, 340 μcool, Eq: 1.0), (4-(4-(tert-butoxycarbonyl)piperazin-1-yl)phenyl)boronic acid (CAS 457613-78-4, 156 mg, 509 μmol, Eq: 1.5) and K₂CO₃ (58.7 mg, 424 μmol, Eq: 1.25) in 2-MeTHF (1.8 mL) and water (0.3 mL) was degassed with argon for 10 min. Dichloro[bis(diphenylphosphinophenyl)ether]palladium(II) (12.2 mg, 17 μmol, Eq: 0.05) was added. The reaction mixture was stirred for 16 h at 80° C. The reaction mixture was poured into EtOAc and washed with water and brine. The organic layer was dried over Na₂SO₄ and concentrated in vacuo. The crude material was purified by flash chromatography (silica gel, 12 g, 0% to 5% MeOH in DCM) to give the title compound as a colorless amorphous solid (88 mg, 38% yield). m/z 653.6 [M+H]⁺, ESI pos.

### Step 2: tert-butyl 4-[4-[4-(difluoromethyl)-7-methyl-2-[2-oxo-1-[(6R)-6-fluoro-6,7-dihydro-5H-pyrrolo[1,2-c]imidazol-1-yl]-2-(thiazol-2-ylamino)ethyl]indazol-6-yl]phenyl]piperazine-1-carboxylate

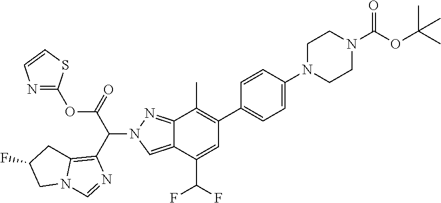 [SMILES: Cc1c(-c2ccc(N3CCN(C(=O)OC(C)(C)C)CC3)cc2)cc(C(F)F)c2cn(C(C(=O)Oc3nccs3)c3ncn4c3C[C@@H](F)C4)nc12]

By analogy with Example 1 Step 2, tert-butyl 4-[4-[4-(difluoromethyl)-2-[2-ethoxy-2-oxo-1-[(6R)-6-fluoro-6,7-dihydro-5H-pyrrolo[1,2-c]imidazol-1-yl]ethyl]-7-methyl-indazol-6-yl]phenyl]piperazine-1-carboxylate (87 mg, 133 μcool, Eq: 1.0) was initially treated with LiOH then aq. HCl, and the resultant carboxylic acid was reacted with 2-aminothiazole in the presence of HATU and DIPEA. Purification by flash chromatography (silica gel, 12 g, 0% to 5% MeOH in DCM) gave the title compound as a yellow solid (69 mg, 92.7 μmol, 70% yield). m/z 707.8 [M+H]⁺, ESI pos.

### Step 3: 2-[4-(difluoromethyl)-7-methyl-6-(4-piperazin-1-ylphenyl)indazol-2-yl]-2-[(6R)-6-fluoro-6,7-dihydro-5H-pyrrolo[1,2-c]imidazol-1-yl]-N-thiazol-2-yl-acetamide Dihydrochloride

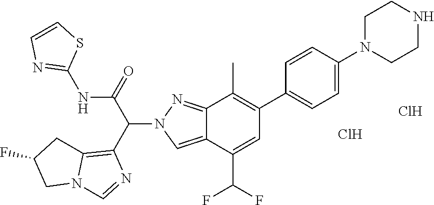 [SMILES: Cc1c(-c2ccc(N3CCNCC3)cc2)cc(C(F)F)c2cn(C(C(=O)Nc3nccs3)c3ncn4c3C[C@@H](F)C4)nc12 ; Cl ; Cl]

To a solution of tert-butyl 4-[4-[4-(difluoromethyl)-7-methyl-2-[2-oxo-1-[r(6R)-6-fluoro-6,7-dihydro-5H-pyrrolo[1,2-c]imidazol-1-yl]-2-(thiazol-2-ylamino)ethyl]indazol-6-yl]phenyl]piperazine-1-carboxylate (66 mg, 93.4 μmol, Eq: 1.0) in DCM (2 mL) and 1,4-dioxane (1 mL) was added HCl (4 M in 1,4-dioxane, 350 μL, 1.4 mmol, Eq: 15). The reaction mixture was stirred for 2 h at rt. The reaction mixture was concentrated in vacuo to give the title compound as an off-white solid (67 mg, 82% purity, 86% yield). m/z 607.2 [M+H]⁺, ESI pos.

### Step 4: 2-[4-(difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

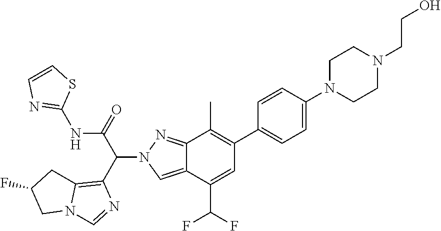 [SMILES: Cc1c(-c2ccc(N3CCN(CCO)CC3)cc2)cc(C(F)F)c2cn(C(C(=O)Nc3nccs3)c3ncn4c3C[C@@H](F)C4)nc12]

To a solution of 2-[4-(difluoromethyl)-7-methyl-6-(4-piperazin-1-ylphenyl)indazol-2-yl]-2-[(6R)-6-fluoro-6,7-dihydro-5H-pyrrolo[1,2-c]imidazol-1-yl]-N-thiazol-2-yl-acetamide dihydrochloride (65 mg, 95.6 μmol, Eq: 1.0) in DMF (1 mL) was added 2-iodoethan-1-ol (19.7 mg, 8.97 μL, 115 μcool, Eq: 1.2) and DIPEA (61.8 mg, 83.5 μL, 478 μmol, Eq: 5.0). The reaction mixture was stirred for 16 h at 55° C. The reaction mixture was poured into EtOAc/THF 2:1 and washed with water and brine. The organic layer was dried over Na₂SO₄ and concentrated in vacuo. The crude material was purified by flash chromatography (silica gel, 12 g, 0% to 30% MeOH in DCM) and freeze-dried to give the title compound as a white solid (22 mg, 34% yield). m/z 651.3 [M+H]+, ESI pos.

### Example 21

### 2-[4-(Difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide

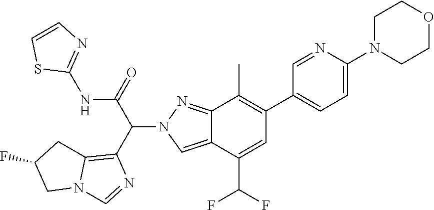 [SMILES: Cc1c(-c2ccc(N3CCOCC3)nc2)cc(C(F)F)c2cn(C(C(=O)Nc3nccs3)c3ncn4c3C[C@@H](F)C4)nc12]

### Step 1: Ethyl 2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]acetate

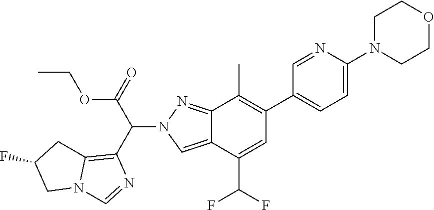 [SMILES: CCOC(=O)C(c1ncn2c1C[C@@H](F)C2)n1cc2c(C(F)F)cc(-c3ccc(N4CCOCC4)nc3)c(C)c2n1]

By analogy with Example 2 Step 7, Intermediate 1 (100 mg, 212 μmol, Eq: 1.0) and (6-morpholinopyridin-3-yl)boronic acid (CAS 904326-93-8, 55.2 mg, 265 μmol, Eq: 1.25) were reacted in the presence of Cs₂CO₃ and Pd(dppf)Cl₂·CH₂Cl₂ for 2 h at 90° C. Purification by flash chromatography (silica gel, 40 g, EtOAc isocratic) to give the title compound as a light brown solid (74 mg, 62% yield). m/z 376.2 [M+H]+, ESI pos.

### Step 2: 2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide

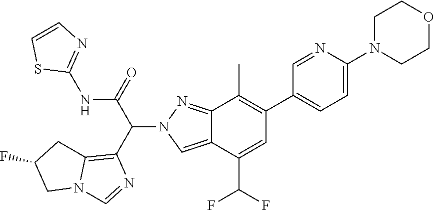 [SMILES: Cc1c(-c2ccc(N3CCOCC3)nc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]acetate (76.3 mg, 138 μmol, Eq: 1.0) was initially treated with LiOH, and the resultant salt was reacted with 2-aminothaizole in the presence of HATU and DIPEA. Purification by flash chromatography (Si-amine, 25 g, 0% to 20% MeOH in EtOAc) gave the title compound as a light brown solid (42 mg, 50% yield). m/z 609.3 [M+H]⁺, ESI pos.

### Example 22

### 2-[4-(Difluoromethyl)-6-[4-[2-[4-(1-hydroxyethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

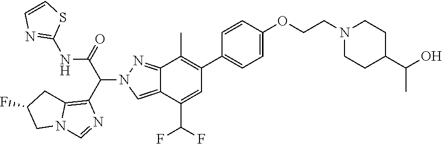 [SMILES: Cc1c(-c2ccc(OCCN3CCC(C(C)O)CC3)cc2)cc(C(F)F)c2cn(C(C(=O)Nc3nccs3)c3ncn4c3C[C@@H](F)C4)nc12]

### Step 1: 1-[1-[2-[4-(4,4,5,5-tetramethyl-1,3,2-dioxaborolan-2-yl)phenoxy]ethyl]-4-piperidyl]ethanol

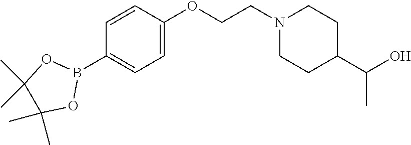 [SMILES: CC(O)C1CCN(CCOc2ccc(B3OC(C)(C)C(C)(C)O3)cc2)CC1]

By analogy with Example 5 Step 1, 1-[1-[2-(4-bromophenoxy)ethyl]-4-piperidyl]ethanol (620 mg, 1.89 mmol, Eq: 1.0, CAS 1919662-84-2) was reacted with bis(pinacolato)diboron in the presence of KOAc and Pd(dppf)Cl₂·CH₂Cl₂ for 1.5 h at 90° C. Purification by flash chromatography (silica gel, 12 g, 0% to 20% MeOH in DCM) to give the title compound as a brown oil (485 mg, 65% yield). m/z 362.2 [M+H]⁺, ESI pos.

### Step 2: Ethyl 2-[4-(difluoromethyl)-6-[4-[2-[4-(1-hydroxyethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]acetate

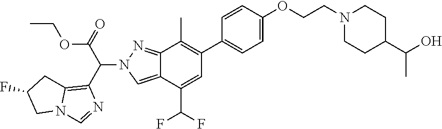 [SMILES: CCOC(=O)C(c1ncn2c1C[C@@H](F)C2)n1cc2c(C(F)F)cc(-c3ccc(OCCN4CCC(C(C)O)CC4)cc3)c(C)c2n1]

By analogy with Example 2 Step 7, Intermediate 1 (100 mg, 212 μmol, Eq: 1.0) and 1-[1-[2-[4-(4,4,5,5-tetramethyl-1,3,2-dioxaborolan-2-yl)phenoxy]ethyl]-4-piperidyl]ethanol (104 mg, 276 μcool, Eq: 1.3) were reacted in the presence of Cs₂CO₃ and Pd(dppf)Cl₂·CH₂Cl₂ at 65° C. for 18 h. Purification by flash chromatography (silica gel, 12 g, 0% to 10% MeOH in DCM) gave the title compound as a yellow solid (53 mg, 36% yield). m/z 640.4 [M+H]⁺, ESI pos.

### Step 3: 2-[4-(difluoromethyl)-6-[4-[2-[4-(1-hydroxyethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

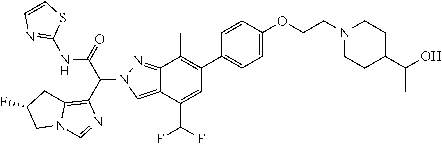 [SMILES: Cc1c(-c2ccc(OCCN3CCC(C(C)O)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[2-[4-(1-hydroxyethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]acetate (52 mg, 81.3 μmol, Eq: 1.0) was initially treated with LiOH, and the resulting salt was reacted with 2-aminothiazole in the presence of HATU and DIPEA. Purification by flash chromatography (silica gel, 12 g, 0% to 20% MeOH in DCM+0.5% NH₄OH) gave the title compound as a light yellow solid (10 mg, 18% yield). m/z 694.3 [M+H]⁺, ESI pos.

### Example 23

### 2-[4-(Difluoromethyl)-6-[4-[1-(2-fluoropropyl)-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

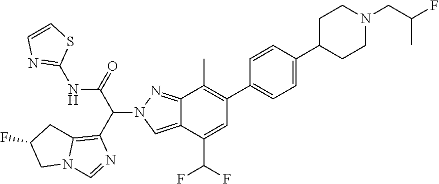 [SMILES: Cc1c(-c2ccc(C3CCN(CC(C)F)CC3)cc2)cc(C(F)F)c2cn(C(C(=O)Nc3nccs3)c3ncn4c3C[C@@H](F)C4)nc12]

### Step 1: 1-[4-(4-bromophenyl)-1-piperidyl]propan-2-ol

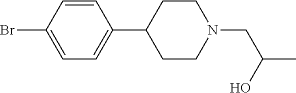 [SMILES: CC(O)CN1CCC(c2ccc(Br)cc2)CC1]

To a solution of 4-(4-bromophenyl)piperidine (CAS 80980-89-8, 10 g, 41.6 mmol, Eq: 1.0) in MeOH (100 mL) was added 2-methyloxirane (CAS 75-56-9, 2.65 g, 3.2 mL, 45.7 mmol, Eq: 1.1). The reaction mixture was stirred at 70° C. overnight. The reaction mixture was concentrated in vacuo and purified by flash chromatography (silica gel, 330 g, 0% to 5% MeOH in DCM) to give the title compound as an off-white solid (7.94 g, 61% yield). m/z 298.1 [{⁷⁹Br}M+H]⁺, 300.1 [{⁸¹Br}M+H]⁺, ESI pos.

### Step 2: 4-(4-bromophenyl)-1-(2-fluoropropyl)piperidine

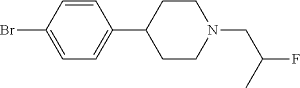 [SMILES: CC(F)CN1CCC(c2ccc(Br)cc2)CC1]

A solution of 1-[4-(4-bromophenyl)-1-piperidyl]propan-2-ol (7.9411 g, 25.3 mmol, Eq: 1.0) in DCM (150 mL) was cooled to −76° C. Deoxofluor (CAS 202289-38-1, 12.3 g, 13.7 mL, 27.8 mmol, Eq: 1.1) was added dropwise at −76° C. The reaction mixture was allowed to slowly warm to rt and stirred at rt overnight. The reaction mixture was quenched with sat. aq. NaHCO₃ and washed with 140 mL sat. aq. NaHCO₃. The aqueous layer was back extracted twice with 150 mL EtOAc. The organic layers were combined, dried over Na₂SO₄, filtered and concentrated in vacuo. The crude material was purified by flash chromatography (silica gel, 330 g, 0% to 50% MeOH in EtOAc) to give the title compound as a yellow liquid (6.18 g, 79% yield). m/z 300.1 [{⁷⁹Br}M+H]⁺, 302.1 [{⁸¹Br}M+H]⁺, ESI pos.

### Step 3: 1-(2-fluoropropyl)-4-[4-(4,4,5,5-tetramethyl-1,3,2-dioxaborolan-2-yl)phenyl]piperidine

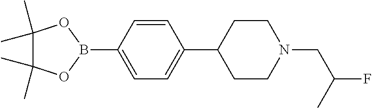 [SMILES: CC(F)CN1CCC(c2ccc(B3OC(C)(C)C(C)(C)O3)cc2)CC1]

By analogy with Example 5 Step 1, 4-(4-bromophenyl)-1-(2-fluoropropyl)piperidine (1.5 g, 5 mmol, Eq: 1.0) was reacted with bis(pinacolato)diboron in the presence of KOAc and Pd(dppf)Cl₂·CH₂Cl₂ for 3 h at 90° C. Purification by flash chromatography (silica gel, 25 g, 0% to 30% EtOAc in heptane) gave the title compound as a dark brown oil (1.45 g, 77% yield). m/z 348.1 [M+H]⁺, ESI pos.

### Step 4: Ethyl 2-[4-(difluoromethyl)-6-[4-[1-(2-fluoropropyl)-4-piperidyl]phenyl]-7-methyl-indazol-2-yl]-2-[(6R)-6-fluoro-6,7-dihydro-5H-pyrrolo[1,2-c]imidazol-1-yl]acetate

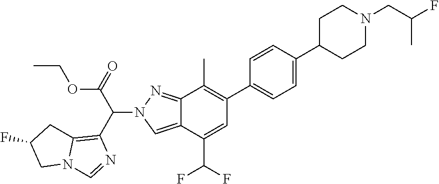 [SMILES: CCOC(=O)C(c1ncn2c1C[C@@H](F)C2)n1cc2c(C(F)F)cc(-c3ccc(C4CCN(CC(C)F)CC4)cc3)c(C)c2n1]

By analogy with Example 2 Step 7, Intermediate 1 (200 mg, 424 μmol, Eq: 1.0) and 1-(2-fluoropropyl)-4-(4-(4,4,5,5-tetramethyl-1,3,2-dioxaborolan-2-yl)phenyl)piperidine (203 mg, 467 μmol Eq: 1.1) were reacted in the presence of Cs₂CO₃ and Pd(dppf)Cl₂·CH₂Cl₂ for 1 hat 100° C. Purification by flash chromatography (silica gel, 25 g, 0% to 10% MeOH in EtOAc) gave the title compound as a brown oil (144 mg, 94% purity, 52% yield). m/z 612.4 [M+H]⁺, ESI pos.

### Step 5: 2-[4-(difluoromethyl)-6-[4-[1-(2-fluoropropyl)-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

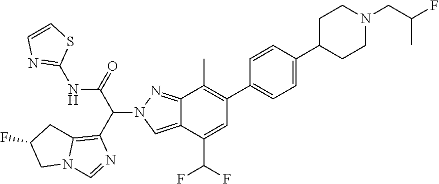 [SMILES: Cc1c(-c2ccc(C3CCN(CC(C)F)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[1-(2-fluoropropyl)-4-piperidyl]phenyl]-7-methyl-indazol-2-yl]-2-[(6R)-6-fluoro-6,7-dihydro-5H-pyrrolo[1,2-c]imidazol-1-yl]acetate (71 mg, 116 μcool, Eq: 1.0) in ethanol (3 mL) was initially treated with LiOH, and the resulting salt was reacted with 2-aminothiazole in the presence of HATU and DIPEA for 2 h at rt then 1 h at 50° C. Purification by flash chromatography (Si-amine, 12 g, 0% to 10% MeOH in EtOAc) gave the title compound as a brown solid (36 mg, 90% purity, 42% yield). m/z 666.3 [M+H]⁺, ESI pos.

### Example 24

### 2-[4-(Difluoromethyl)-6-[4-(4-hydroxy-4-methyl-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

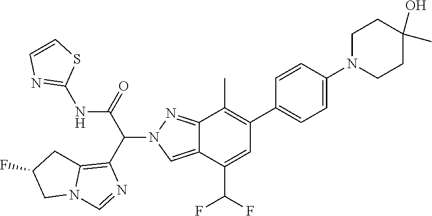 [SMILES: Cc1c(-c2ccc(N3CCC(C)(O)CC3)cc2)cc(C(F)F)c2cn(C(C(=O)Nc3nccs3)c3ncn4c3C[C@@H](F)C4)nc12]

### Step 1: 1-(4-bromophenyl)-4-methyl-piperidin-4-ol

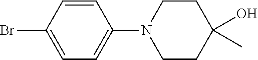

By analogy with Example 13 Step 1, 1-bromo-4-iodobenzene (1 g, 3.53 mmol, Eq: 1.0) and 4-methylpiperidin-4-ol (407 mg, 3.53 mmol, Eq: 1.0) were reacted in the presence of NaO^{t}Bu, XantPhos (205 mg, 353 μmol, Eq: 0.10) and Pd₂(dba)₃ for 1.5 h at 85° C. Purification by flash chromatography (silica gel, 40 g, 0% to 40% EtOAc in heptane) gave the title compound as a yellow solid (900 mg, 90% purity, 85% yield). m/z 271.7 [M+H]⁺, ESI pos.

### Step 2: 4-methyl-1-[4-(4,4,5,5-tetramethyl-1,3,2-dioxaborolan-2-yl)phenyl]piperidin-4-ol

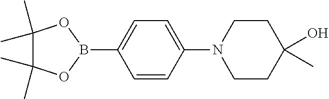

By analogy with Example 5 Step 1, 1-(4-bromophenyl)-4-methylpiperidin-4-ol (605 mg, 2.24 mmol, Eq: 1.0) was reacted with bis(pinacolato)diboron in the presence of KOAc and Pd(dppf)Cl₂·CH₂Cl₂ for 2 h at 90° C. Purification by flash chromatography (silica gel, 24 g, 0% to 40% EtOAc in heptane) gave the title compound as an off-white powder (560 mg, 79% yield). m/z 318.2 [M+H]⁺, ESI pos.

### Step 3: Ethyl 2-[4-(difluoromethyl)-6-[4-(4-hydroxy-4-methyl-1-piperidyl)phenyl]-7-methyl-indazol-2-yl]-2-[(6R)-6-fluoro-6,7-dihydro-5H-pyrrolo[1,2-c]imidazol-1-yl]acetate

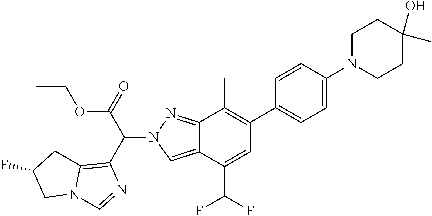 [SMILES: CCOC(=O)C(c1ncn2c1C[C@@H](F)C2)n1cc2c(C(F)F)cc(-c3ccc(N4CCC(C)(O)CC4)cc3)c(C)c2n1]

By analogy with Example 2 Step 7, Intermediate 1 (100 mg, 212 μmol, Eq: 1.0) and 4-methyl-1-[4-(4,4,5,5-tetramethyl-1,3,2-dioxaborolan-2-yl)phenyl]piperidin-4-ol (101 mg, 318 μmol, Eq: 1.5) were reacted in the presence of Cs₂CO₃ and Pd(dppf)Cl₂·CH₂Cl₂ for 3 h at 65° C. Purification by flash chromatography (silica gel, 12 g, 0% to 10% MeOH in EtOAc) gave the title compound as a light brown solid (105 mg, 85% yield). m/z 582.3 [M+H]+, ESI pos.

### Step 4: 2-[4-(difluoromethyl)-6-[4-(4-hydroxy-4-methyl-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

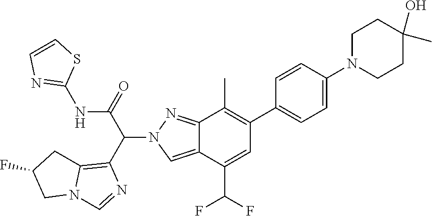 [SMILES: Cc1c(-c2ccc(N3CCC(C)(O)CC3)cc2)cc(C(F)F)c2cn(C(C(=O)Nc3nccs3)c3ncn4c3C[C@@H](F)C4)nc12]

By analogy with Example 8 Step 2, ethyl 2-[4-(difluoromethyl)-6-[4-(4-hydroxy-4-methyl-1-piperidyl)phenyl]-7-methyl-indazol-2-yl]-2-[(6R)-6-fluoro-6,7-dihydro-5H-pyrrolo[1,2-c]imidazol-1-yl]acetate (105 mg, 181 μcool, Eq: 1.0) was initially treated with LiOH then aq. HCl, and the resulting carboxylic acid was reacted with 2-aminothiazole in the presence of HATU and DIPEA. Purification by flash chromatography (silica gel, 12 g, 0% to 10% MeOH in EtOAc) gave the title compound as a light brown solid (49.2 mg, 61% yield). m/z 636.2 [M+H]⁺, ESI pos.

### Example 25

### 2-[6-[4-[(3aR,6aS)-2-Ethyl-3a-fluoro-3,4,6,6a-tetrahydro-1H-pyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

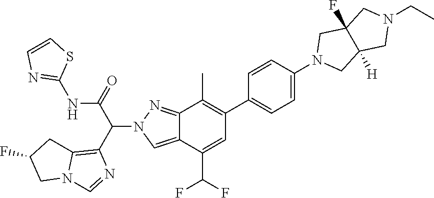 [SMILES: CCN1C[C@H]2CN(c3ccc(-c4cc(C(F)F)c5cn(C(C(=O)Nc6nccs6)c6ncn7c6C[C@@H](F)C7)nc5c4C)cc3)C[C@]2(F)C1]

### Step 1: Tert-Butyl (3 aS,6aR)-2-(4-bromophenyl)-3a-fluoro-3,4,6,6a-tetrahydro-1H-pyrrolo[3,4-c]pyrrole-5-carboxylate

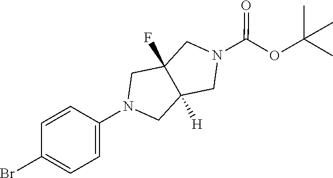 [SMILES: CC(C)(C)OC(=O)N1C[C@H]2CN(c3ccc(Br)cc3)C[C@]2(F)C1]

By analogy with Example 13 Step 1, 1-bromo-4-iodobenzene (622 mg, 2.2 mmol, Eq: 1.1) and tert-butyl (3 aS, 6aR)-3a-fluorohexahydropyrrolo[3,4-c]pyrrole-2(1H)-carboxylate (CAS 2090176-68-2, 500 mg, 2 mmol, Eq: 1.0) were reacted in the presence of NaO^{t}Bu, XantPhos and Pd₂(dba)₃·CHCl₃ at 80° C. overnight. Purification by flash chromatography (silica gel, 40 g, 0% to 30% EtOAc in heptane) to give the title compound as a light brown solid (308 mg, 38% yield). m/z 387.1 [M+H]⁺, ESI pos.

### Step 2: Tert-Butyl (3aS,6aR)-3a-fluoro-2-[4-(4,4,5,5-tetramethyl-1,3,2-dioxaborolan-2-yl)phenyl]-3,4,6,6a-tetrahydro-1H-pyrrolo[3,4-c]pyrrole-5-carboxylate

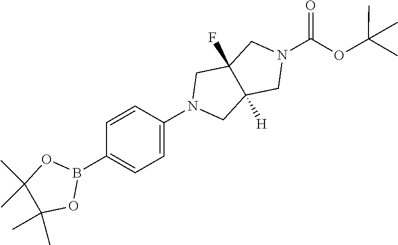 [SMILES: CC(C)(C)OC(=O)N1C[C@H]2CN(c3ccc(B4OC(C)(C)C(C)(C)O4)cc3)C[C@]2(F)C1]

By analogy with Example 5 Step 1, tert-butyl (3 aS,6aR)-2-(4-bromophenyl)-3a-fluoro-3,4,6,6a-tetrahydro-1H-pyrrolo[3,4-c]pyrrole-5-carboxylate (0.300 g, 740 μmol, Eq: 1.0) was reacted with bis(pinacolato)diboron in the presence of KOAc and Pd(dppf)Cl₂·CH₂Cl₂ at 90° C. for 6 h. Purification by flash chromatography (silica gel, 12 g, 0% to 20% EtOAc in heptane) gave the title compound as a light yellow solid (117 mg, 90% purity, 32% yield). m/z 433.3 [M+H]⁺, ESI pos.

### Step 3: tert-butyl (3aS,6aR)-2-[4-[4-(difluoromethyl)-2-[2-ethoxy-1-[(6R)-6-fluoro-6,7-dihydro-5H-pyrrolo[1,2-c]imidazol-1-yl]-2-oxo-ethyl]-7-methyl-indazol-6-yl]phenyl]-3a-fluoro-3,4,6,6a-tetrahydro-1 H-pyrrolo[3,4-e]pyrrole-5-carboxylate

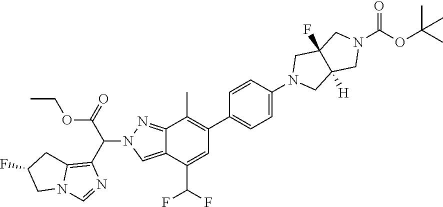 [SMILES: CCOC(=O)C(c1ncn2c1C[C@@H](F)C2)n1cc2c(C(F)F)cc(-c3ccc(N4C[C@@H]5CN(C(=O)OC(C)(C)C)C[C@@]5(F)C4)cc3)c(C)c2n1]

By analogy with Example 2 Step 7, Intermediate 1 (115 mg, 244 μmol, Eq: 1.0) and tert-butyl (3 aS, 6aR)-3a-fluoro-2-[4-(4,4,5,5-tetramethyl-1,3,2-dioxaborolan-2-yl)phenyl]-3,4,6,6a-tetrahydro-1H-pyrrolo[3,4-c]pyrrole-5-carboxylate (110 mg, 254 μcool, Eq: 1.04) were reacted in the presence of Cs₂CO₃ and Pd(dppf)Cl₂·CH₂Cl₂ at 70° C. for 2.5 h. Purification by flash chromatography (silica gel, 25 g, 0% to 100% (DCM:MeOH:NH₄OH 9:1:0.05) in DCM) gave the title compound as a brown oil (110 mg, 60% purity, 38% yield). m/z 697.7 [M+H]⁺, ESI pos.

### Step 4: Tert-Butyl (3aS,6aR)-2-[4-[4-(difluoromethyl)-2-[1-[(6R)-6-fluoro-6,7-dihydro-5H-pyrrolo[1,2-c]imidazol-1-yl]-2-oxo-2-(thiazol-2-ylamino)ethyl]-7-methyl-indazol-6-yl]phenyl]-3a-fluoro-3,4,6,6a-tetrahydro-1H-pyrrolo[3,4-c]pyrrole-5-carboxylate

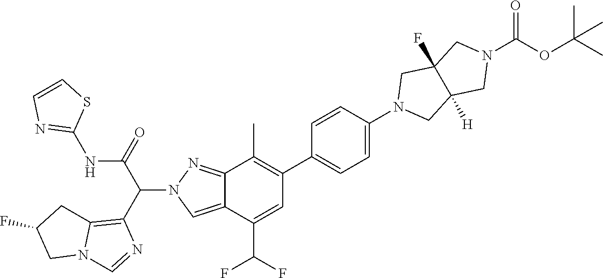 [SMILES: Cc1c(-c2ccc(N3C[C@@H]4CN(C(=O)OC(C)(C)C)C[C@@]4(F)C3)cc2)cc(C(F)F)c2cn(C(C(=O)Nc3nccs3)c3ncn4c3C[C@@H](F)C4)nc12]

By analogy with Example 2 Step 8, tert-butyl (3aS,6aR)-2-[4-[4-(difluoromethyl)-2-[2-ethoxy-1-[(6R)-6-fluoro-6,7-dihydro-5H-pyrrolo[1,2-c]imidazol-1-yl]-2-oxo-ethyl]-7-methyl-indazol-6-yl]phenyl]-3a-fluoro-3,4,6,6a-tetrahydro-1H-pyrrolo[3,4-c]pyrrole-5-carboxylate (110 mg, 158 μmol Eq: 1.0) was initially treated with LiOH, and the resulting salt was reacted with 2-aminothiazole in the presence of HATU and DIPEA. Purification by flash chromatography (silica gel, 12 g, 0% to 10% MeOH in EtOAc) gave the title compound as a brown oil (60 mg, 35% purity, 17% yield). m/z 751.3 [M+H]⁺, ESI pos.

### Step 5: 2-[6-[4-[(3aR,6aS)-3a-fluoro-1,2,3,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

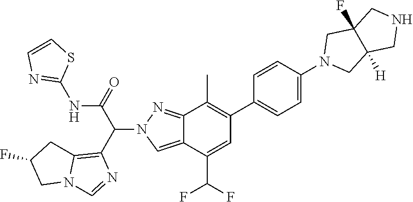 [SMILES: Cc1c(-c2ccc(N3C[C@@H]4CNC[C@@]4(F)C3)cc2)cc(C(F)F)c2cn(C(C(=O)Nc3nccs3)c3ncn4c3C[C@@H](F)C4)nc12]

tert-butyl (3aS,6aR)-2-[4-[4-(difluoromethyl)-2-[1-[(6R)-6-fluoro-6,7-dihydro-5H-pyrrolo[1,2-c]imidazol-1-yl]-2-oxo-2-(thiazol-2-ylamino)ethyl]-7-methyl-indazol-6-yl]phenyl]-3a-fluoro-3,4,6,6a-tetrahydro-1H-pyrrolo[3,4-c]pyrrole-5-carboxylate (0.06 g, 79.9 μcool, Eq: 1.0) was dissolved in 0.3 mL DCM and 0.15 mL MeOH. Then, hydrogen chloride solution (4 M in dioxane, 240 μL, 959 μmol, Eq: 12) was added dropwise. The reaction mixture was stirred at rt for 1.5 h then concentrate in vacuo. The residue was dissolved in a mixture of DCM and MeOH, carefully basified with saturated aq. NaHCO₃ and then extracted three times with a mixture of DCM and MeOH (19:1). The organic layers were combined, dried over Na₂SO₄, filtered and concentrated to give the title compound as a brown oil (60 mg, 40% purity, 46% yield). m/z 651.2 [M+H]⁺, ESI pos.

### Step 6: 2-[6-[4-[(3aR,6aS)-2-ethyl-3a-fluoro-3,4,6,6a-tetrahydro-1H-pyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

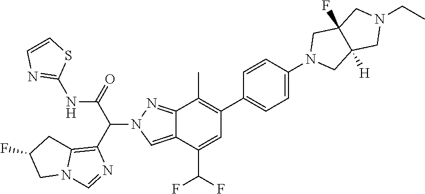 [SMILES: CCN1C[C@H]2CN(c3ccc(-c4cc(C(F)F)c5cn(C(C(=O)Nc6nccs6)c6ncn7c6C[C@@H](F)C7)nc5c4C)cc3)C[C@]2(F)C1]

2-[6-[4-[(3aR,6aS)-3a-fluoro-1,2,3,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide (0.06 g, 64.5 μmol, Eq: 1.0) was dissolved in DMF (2.5 mL), then DIPEA (41.7 mg, 56.4 μL, 323 μmol, Eq: 5.0) and ethyl iodide (13.1 mg, 6.78 μL, 83.9 μmol Eq: 1.3) were added at rt. The mixture was stirred for 2 h at 50° C. The reaction mixture was diluted with sat. aq. NaHCO₃ and extracted two times with EtOAc. The organic layers were washed with water and brine, dried over Na₂SO₄, filtered and evaporated to dryness. The crude material was purified by flash chromatography (silica gel, 12 g, 0% to 100% (DCM:MeOH:NH₄OH 9:1:0.05) in DCM) to give the title compound as a brown oil (15 mg, 87% purity, 34% yield). m/z 679.3 [M+H]⁺, ESI pos.

### Example 26

### 2-[4-(Difluoromethyl)-6-[4-[2-[(dimethylamino)methyl]morpholin-4-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

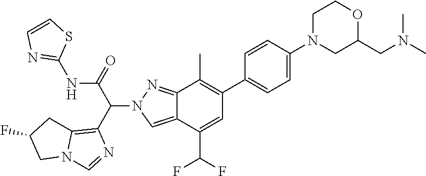 [SMILES: Cc1c(-c2ccc(N3CCOC(CN(C)C)C3)cc2)cc(C(F)F)c2cn(C(C(=O)Nc3nccs3)c3ncn4c3C[C@@H](F)C4)nc12]

### Step 1: 1-[4-(4-bromophenyl)morpholin-2-yl]-N,N-dimethyl-methanamine

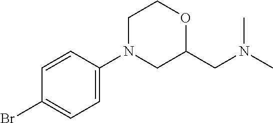 [SMILES: CN(C)CC1CN(c2ccc(Br)cc2)CCO1]

By analogy with Example 13 Step 1, 1-bromo-4-iodo-benzene (1.19 g, 4.2 mmol, Eq: 1.1) and N,N-dimethyl-1-morpholin-2-yl-methanamine (CAS 122894-56-8, 550 mg, 3.81 mmol, Eq: 1.0) were reacted in the presence of NaO^{t}Bu, xantphos and Pd₂(dba)₃ for 6 h at 90° C. and for 16 h at rt. Purification by flash chromatography (silica gel, 40 g, 0% to 100% EtOAc in heptane) gave the title compound as a brown oil (0.77 g, 70% purity, 47% yield). m/z 299.0, 300.8 [M+H]⁺, ESI pos, Br isotopes.

### Step 2: N,N-dimethyl-1-[4-[4-(4,4,5,5-tetramethyl-1,3,2-dioxaborolan-2-yl)phenyl]morpholin-2-yl]methanamine

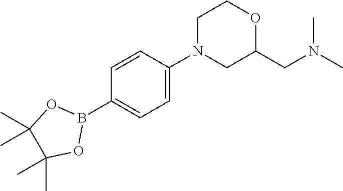

By analogy with Example 5 Step 1, 1-[4-(4-bromophenyl)morpholin-2-yl]-N,N-dimethyl-methanamine (770 mg, 2.57 mmol, Eq: 1.0) was reacted with bis(pinacolato)diboron in the presence of KOAc and Pd(dppf)Cl₂·CH₂Cl₂ for 3 h at 90° C. Purification by flash chromatography (silica gel, 12 g, 0% to 100% (DCM:MeOH:NH₄OH 9:1:0.05) in DCM) gave the title compound as a dark brown viscous oil (96 mg, 80% purity, 9% yield). m/z 347.3 [M+H]⁺, ESI pos.

### Step 3: Ethyl 2-[4-(difluoromethyl)-6-[4-[2-[(dimethylamino)methyl]morpholin-4-yl]phenyl]-7-methyl-indazol-2-yl]-2-[(6R)-6-fluoro-6,7-dihydro-5H-pyrrolo[1,2-c]imidazol-1-yl]acetate

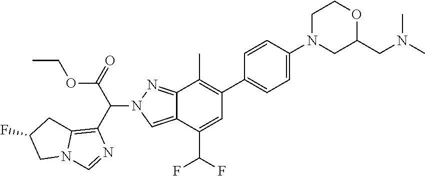 [SMILES: CCOC(=O)C(c1ncn2c1C[C@@H](F)C2)n1cc2c(C(F)F)cc(-c3ccc(N4CCOC(CN(C)C)C4)cc3)c(C)c2n1]

By analogy with Example 2 Step 7, Intermediate 1 (70 mg, 0.149 mmol, Eq: 1.0) and N,N-dimethyl-1-[4-[4-(4,4,5,5-tetramethyl-1,3,2-dioxaborolan-2-yl)phenyl]morpholin-2-yl]methanamine (96 mg, 0.223 mmol, Eq: 1,5, 80% purity) were reacted in the presence of Cs₂CO₃ and Pd(dppf)Cl₂·CH₂Cl₂ for 1.5 h at 65° C. Purification by flash chromatography (silica gel, 12 g, 0% to 10% MeOH in DCM) gave the title compound as a brown solid (70 mg, 65% purity, 50% yield). m/z 611.4 [M+H]⁺, ESI pos.

### Step 4: 2-[4-(difluoromethyl)-6-[4-[2-[(dimethylamino)methyl]morpholin-4-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

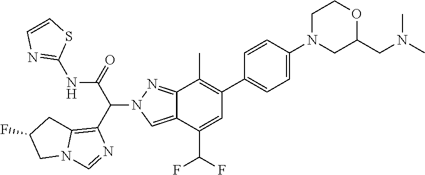 [SMILES: Cc1c(-c2ccc(N3CCOC(CN(C)C)C3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[2-[(dimethylamino)methyl]morpholin-4-yl]phenyl]-7-methyl-indazol-2-yl]-2-[(6R)-6-fluoro-6,7-dihydro-5H-pyrrolo[1,2-c]imidazol-1-yl]acetate (70 mg, 0.075 mmol, Eq: 1.0, 65% purity) was initially treated with LiOH, and the resultant salt was reacted with 2-aminothiazole in the presence of HATU and DIPEA. Purification by flash chromatography (Si-amine, 12 g, 0% to 10% MeOH in EtOAc and then switched to 0% to 20% MeOH in DCM) gave the title compound as a light brown powder (20 mg, 83% purity, 33% yield). m/z 665.3 [M+H]⁺, ESI pos.

### Example 27

### 2-[4-(Difluoromethyl)-6-[4-[2-(4-hydroxy-1-piperidyl)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

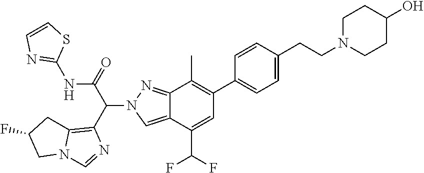 [SMILES: Cc1c(-c2ccc(CCN3CCC(O)CC3)cc2)cc(C(F)F)c2cn(C(C(=O)Nc3nccs3)c3ncn4c3C[C@@H](F)C4)nc12]

### Step 1: 1-[2-(4-bromophenyl)ethyl]piperidin-4-ol

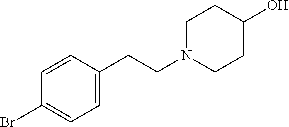 [SMILES: OC1CCN(CCc2ccc(Br)cc2)CC1]

To a mixture of 2-(4-bromophenyl)acetaldehyde (CAS 27200-79-9, 400 mg, 2.01 mmol, Eq: 1.0), piperidin-4-ol (CAS 5382-16-1, 264 mg, 2.61 mmol, Eq: 1.3) in DCM (6.0 mL) was added sodium triacetoxyborohydride (681 mg, 3.22 mmol, Eq: 1.6). The reaction mixture was stirred for 3 h at rt. The reaction mixture was diluted with sat. aq. NaHCO₃ and extracted twice with a mixture of DCM:MeOH (9:1). The combined organic layers were dried over Na₂SO₄ and concentrated in vacuo. The crude material was purified by flash chromatography (Si-amine, 24 g, 0% to 20% MeOH in EtOAc) to give the title compound as a light brown solid (492 mg, 82% yield). m/z 286.1 [M+H]⁺, ESI pos.

### Step 2: 1-[2-[4-(4,4,5,5-tetramethyl-1,3,2-dioxaborolan-2-yl)phenyl]ethyl]piperidin-4-ol

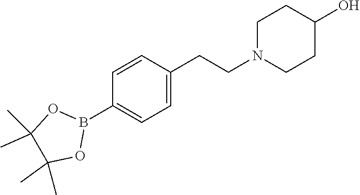 [SMILES: CC1(C)OB(c2ccc(CCN3CCC(O)CC3)cc2)OC1(C)C]

By analogy with Example 5 Step 1, 1-[2-(4-bromophenyl)ethyl]piperidin-4-ol (492 mg, 1.73 mmol, Eq: 1.0) was reacted with bis(pinacolato)diboron in the presence of KOAc and Pd(dppf)Cl₂·CH₂Cl₂ for 1.5 h at 90° C. Purification by flash chromatography (silica gel, 24 g, 0% to 10% MeOH in DCM) gave the title compound as a dark brown oil (226 mg, 37% yield). m/z 332.2 [M+H]⁺, ESI pos.

### Step 3: Ethyl 2-[4-(difluoromethyl)-6-[4-[2-(4-hydroxy-1-piperidyl)ethyl]phenyl]-7-methyl-indazol-2-yl]-2-[(6R)-6-fluoro-6,7-dihydro-5H-pyrrolo[1,2-c]imidazol-1-yl]acetate

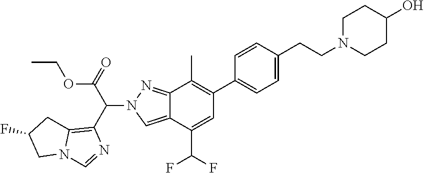 [SMILES: CCOC(=O)C(c1ncn2c1C[C@@H](F)C2)n1cc2c(C(F)F)cc(-c3ccc(CCN4CCC(O)CC4)cc3)c(C)c2n1]

By analogy with Example 2 Step 7, Intermediate 1 (150 mg, 0.318 mmol, Eq: 1.0) and 1-[2-[4-(4,4,5,5-tetramethyl-1,3,2-dioxaborolan-2-yl)phenyl]ethyl]piperidin-4-ol (137 mg, 0.414 mmol, Eq: 1.3) were reacted in the presence of Cs₂CO₃ and Pd(dppf)Cl₂·CH₂Cl₂ for 2 h at 65° C. Purification by flash chromatography (silica gel, 12 g, 0% to 10% MeOH in DCM) gave the title compound as an off-white solid (128 mg, 67% yield). m/z 596.3 [M+H]⁺, ESI pos.

### Step 4: 2-[4-(difluoromethyl)-6-[4-[2-(4-hydroxy-1-piperidyl)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

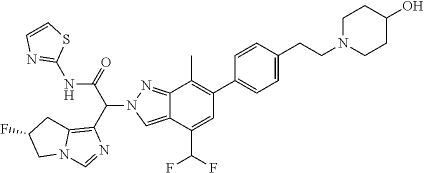 [SMILES: Cc1c(-c2ccc(CCN3CCC(O)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[2-(4-hydroxy-1-piperidyl)ethyl]phenyl]-7-methyl-indazol-2-yl]-2-[(6R)-6-fluoro-6,7-dihydro-5H-pyrrolo[1,2-c]imidazol-1-yl]acetate (128 mg, 0.214 mmol, Eq: 1.0) was initially treated with LiOH, and the resulting salt was reacted with 2-aminothiazole in the presence of HATU and DIPEA. Purification by flash chromatography (silica gel, 12 g, 0% to 20% MeOH in DCM) gave the title compound as a yellow solid (32 mg, 23% yield). m/z 650.3 [M+H]⁺, ESI pos.

### Example 28

### 2-[4-(Difluoromethyl)-6-[4-[[(4-hydroxycyclohexyl)-methyl-amino]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

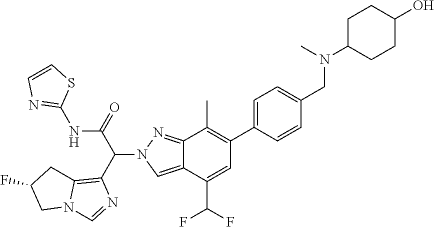 [SMILES: Cc1c(-c2ccc(CN(C)C3CCC(O)CC3)cc2)cc(C(F)F)c2cn(C(C(=O)Nc3nccs3)c3ncn4c3C[C@@H](F)C4)nc12]

### Step 1: [4-[[(4-hydroxycyclohexyl)-methyl-amino]methyl]phenyl]boronic Acid

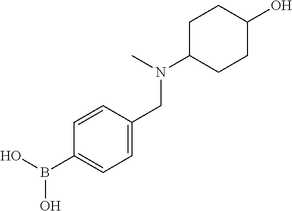 [SMILES: CN(Cc1ccc(B(O)O)cc1)C1CCC(O)CC1]

To a suspension of (4-formylphenyl)boronic acid (CAS 87199-17-5, 200 mg, 1.33 mmol, Eq: 1.0) and 4-(methylamino)cyclohexan-1-ol (CAS 2987-05-5, 172 mg, 1.33 mmol, Eq: 1.0) in dichloromethane (10 mL) was added acetic acid (160 mg, 153 μL, 2.67 mmol, Eq: 2.0) at rt. The reaction mixture was heated at 40° C. for 1 h. Sodium triacetoxyborohydride (565 mg, 2.67 mmol, Eq: 2.0) was added at rt. The reaction mixture was stirred overnight at rt, then further sodium triacetoxyborohydride (565 mg, 2.67 mmol, Eq: 2.0) was added, and the reaction mixture was stirred for 3 h. The reaction mixture was diluted with 1 M aq. NaHCO₃ (10 mL). The layers were separated. The aqueous layer was extracted with two 10-ml portions of dichloromethane. The aqueous layer was concentrated in vacuo to give a white solid (1.8 g) containing the title compound as well as NaHCO₃ and salts. This was used without further purification in the next step. m/z 264.2 [M+H]⁺, ESI pos.

### Step 2: ethyl 2-[4-(difluoromethyl)-6-[4-[[(4-hydroxycyclohexyl)-methyl-amino]methyl]phenyl]-7-methyl-indazol-2-yl]-2-[(6R)-6-fluoro-6,7-dihydro-5H-pyrrolo[1,2-c]imidazol-1-yl]acetate

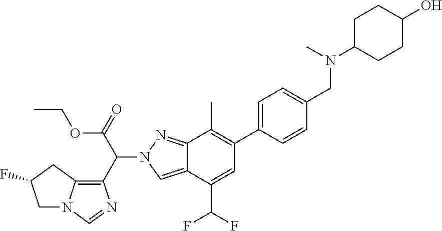 [SMILES: CCOC(=O)C(c1ncn2c1C[C@@H](F)C2)n1cc2c(C(F)F)cc(-c3ccc(CN(C)C4CCC(O)CC4)cc3)c(C)c2n1]

In a vial, Intermediate 1 (150 mg, 318 μmol, Eq: 1.0), impure [4-[[(4-hydroxycyclohexyl)-methyl-amino]methyl]phenyl]boronic acid (251 mg), Cs₂CO₃ (311 mg, 955 μmol, Eq: 3.0) and Pd(dppf)Cl₂·CH₂Cl₂ (26 mg, 31.8 μmol, Eq: 0.10) were combined with THE (3.6 mL) and water (600 μL). The red solution was heated to 65° C. under argon and stirred for 18 h overnight. As the reaction was not yet completed, further impure [4-[[(4-hydroxycyclohexyl)-methyl-amino]methyl]phenyl]boronic acid (251 mg) was added, and the reaction was stirred at 65° C. under argon for 18 h. The reaction mixture was diluted with water and extracted two times with EtOAc. The organic layers were washed with water and brine, dried over Na₂SO₄ and concentrated to dryness. The crude material was purified by flash chromatography (silica gel, 12 g, 0% to 10% MeOH in DCM) to give the title compound as a dark brown oil (52 mg, 70% purity, 19% yield). m/z 610.3 [M+H]⁺, ESI pos.

### Step 3: 2-[4-(difluoromethyl)-6-[4-[[(4-hydroxycyclohexyl)-methyl-amino]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide

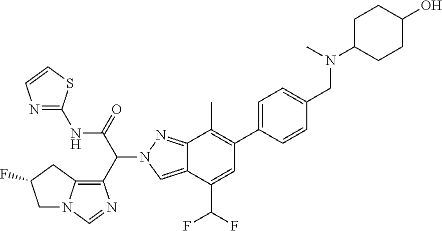 [SMILES: Cc1c(-c2ccc(CN(C)C3CCC(O)CC3)cc2)cc(C(F)F)c2cn(C(C(=O)Nc3nccs3)c3ncn4c3C[C@@H](F)C4)nc12]

By analogy with Example 2 Step 8, ethyl 2-[4-(difluoromethyl)-6-[4-[[(4-hydroxycyclohexyl)-methyl-amino]methyl]phenyl]-7-methyl-indazol-2-yl]-2-[(6R)-6-fluoro-6,7-dihydro-5H-pyrrolo[1,2-c]imidazol-1-yl]acetate (52 mg, 85.3 μcool, Eq: 1.0) was initially treated with LiOH, and the resulting salt was reacted with 2-aminothiazole in the presence of HATU and DIPEA. Purification by flash chromatography (silica gel, 12 g, 0% to 20% MeOH in DCM) gave the title compound as a dark brown solid (9.7 mg, 16% yield). m/z 664.4 [M+H]⁺, ESI pos.

### Example 29

### 2-[6-[4-[(3aR,6aS)-2-(2-Fluoropropyl)-1,3,3a,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

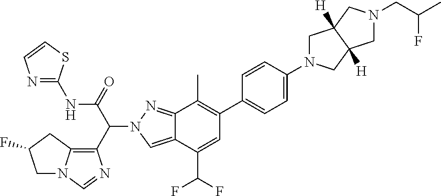 [SMILES: Cc1c(-c2ccc(N3C[C@H]4CN(CC(C)F)C[C@H]4C3)cc2)cc(C(F)F)c2cn(C(C(=O)Nc3nccs3)c3ncn4c3C[C@@H](F)C4)nc12]

### Step 1: tert-butyl (3 aR,6aS)-2-(4-bromophenyl)-1,3,3a,4,6,6a-hexahydropyrrolo[3,4-c]pyrrole-5-carboxylate

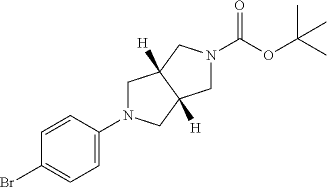 [SMILES: CC(C)(C)OC(=O)N1C[C@@H]2CN(c3ccc(Br)cc3)C[C@@H]2C1]

By analogy with Example 13 Step 1, 1-bromo-4-iodo-benzene (1.47 g, 5.18 mmol, Eq: 1.1) and tert-butyl (3aR,6aS)-2,3,3a,4,6,6a-hexahydro-1H-pyrrolo[3,4-c]pyrrole-5-carboxylate (CAS 250275-15-1, 1.00 g, 4.71 mmol, Eq: 1.0) were reacted in the presence of NaO^{t}Bu, xantphos and Pd₂(dba)₃·CHCl₃ for 16 h at 80° C. Purification by flash chromatography (silica gel, 80 g, 0% to 30% EtOAc in heptane) gave the title compound as a brown solid (1.44 g, 79% yield). m/z 367.1, 369.1 [M+H]⁺, ESI pos, Br isotopes.

### Step 2: (3aR,6aS)-5-(4-bromophenyl)-2,3,3a,4,6,6a-hexahydro-1H-pyrrolo[3,4-c]pyrrole

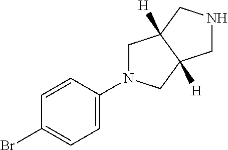 [SMILES: Brc1ccc(N2C[C@H]3CNC[C@H]3C2)cc1]

To a solution of tert-butyl (3 aR,6aS)-2-(4-bromophenyl)-1,3,3a,4,6,6a-hexahydropyrrolo[3,4-c]pyrrole-5-carboxylate (850 mg, 2.2 mmol, Eq: 1.0) in DCM (10 mL) and MeOH (5.0 mL) was added dropwise HCl (4 M in 1,4-dioxane, 5.0 mL, 20 mmol, Eq: 9.1). The reaction mixture was stirred for 16 h at rt. The reaction mixture was concentrated in vacuo. The residue was dissolved in DCM/MeOH, basified with sat. aq. NaHCO₃ and then extracted four times with a mixture of DCM:MeOH (9:1). The combined organic layers were dried over Na₂SO₄ and concentrated in vacuo to give the title compound as a brown solid (693 mg, 80% purity, 94% yield), which was used in the next step without further purification. m/z 267.0, 268.7 [M+H]⁺, ESI pos, Br isotopes.

### Step 3: 1-[(3aR,6aS)-5-(4-bromophenyl)-1,3,3a,4,6,6a-hexahydropyrrolo[3,4-c]pyrrol-2-yl]propan-2-ol

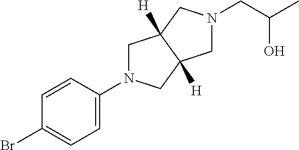 [SMILES: CC(O)CN1C[C@@H]2CN(c3ccc(Br)cc3)C[C@@H]2C1]

To a solution of (3aR,6aS)-5-(4-bromophenyl)-2,3,3a,4,6,6a-hexahydro-1H-pyrrolo[3,4-c]pyrrole (685 mg, 2.05 mmol, Eq: 1.0, 80% purity) in MeOH (5.4 mL) was added 2-methyloxirane (CAS 75-56-9, 141 mg, 0.17 mL, 2.43 mmol, Eq: 1.18). The reaction mixture was stirred for 16 h at 60° C. The reaction mixture was cooled to rt, adsorbed on Isolute HM-N and purified by flash chromatography (silica gel, 40 g, 0% to 5% MeOH in DCM) to give the title compound as an off-white solid (495 mg, 71% yield). m/z 325.1, 327.1 [M+H]⁺, ESI pos, Br isotopes.

### Step 4: (3 aR,6aS)-5-(4-bromophenyl)-2-(2-fluoropropyl)-1,3,3a,4,6,6a-hexahydropyrrolo[3,4-c]pyrrole

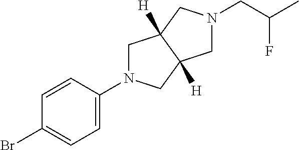 [SMILES: CC(F)CN1C[C@@H]2CN(c3ccc(Br)cc3)C[C@@H]2C1]

A solution of 1-[(3aR,6aS)-5-(4-bromophenyl)-1,3,3a,4,6,6a-hexahydropyrrolo[3,4-c]pyrrol-2-yl]propan-2-ol (480 mg, 1.4 mmol, Eq: 1.0) in DCM (8.0 mL) was cooled to −76° C. Deoxofluor® (2.7 M solution in toluene, CAS 202289-38-1, 0.62 mL, 1.67 mmol, Eq: 1.19) was added dropwise at −76° C. The reaction mixture was allowed to slowly warm to rt and stirred for 16 h at rt. The reaction mixture was quenched with sat. aq. NaHCO₃ and extracted three times with DCM. The combined organic layers were dried over Na₂SO₄ and concentrated in vacuo. The crude material was adsorbed on Isolute HM-N and purified by flash chromatography (silica gel, 24 g, 0% to 70% EtOAc in heptane) to give the title compound as a light yellow solid (275 mg, 57% yield)). m/z 327.0, 328.9 [M+H]⁺, ESI pos, Br isotopes.

### Step 5: [4-[(3aR,6aS)-2-(2-fluoropropyl)-1,3,3a,4,6,6a-hexahydropyrrolo[3,4-c]pyrrol-5-yl]phenyl]boronic Acid

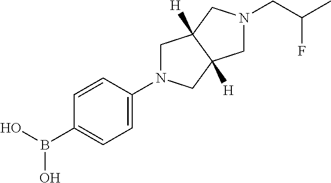 [SMILES: CC(F)CN1C[C@@H]2CN(c3ccc(B(O)O)cc3)C[C@@H]2C1]

A solution of (3 aR,6aS)-5-(4-bromophenyl)-2-(2-fluoropropyl)-1,3,3a,4,6,6a-hexahydropyrrolo[3,4-c]pyrrole (264 mg, 0.766 mmol, Eq: 1.0) in THE (3.0 mL) was cooled to 76° C. n-Butyllithium (1.6 M solution in hexanes, 0.53 mL, 0.848 mmol, Eq: 1.11) was added and the reaction mixture was stirred for 1.5 h at −76° C. Triethyl borate (154 mg, 0.18 mL, 1.06 mmol, Eq: 1.38) was added at −76° C. and the reaction mixture was stirred for 15 min at −76° C. Then, the dry ice bath was removed and the reaction mixture was stirred at rt for 1 h. The reaction mixture was quenched with sat. aq. NH₄Cl (5 mL) and stirred for 30 min at rt. The mixture was extracted twice with EtOAc. The organic layers were washed with water and brine, dried over Na₂SO₄ and concentrated in vacuo to give the title compound as a brown solid (223 mg, 85% purity, 85% yield) which was used in the next step without further purification. m/z 292.8 [M+H]⁺, ESI pos.

### Step 6: Ethyl 2-[6-[4-[(3aR,6aS)-2-(2-fluoropropyl)-1,3,3a,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]acetate

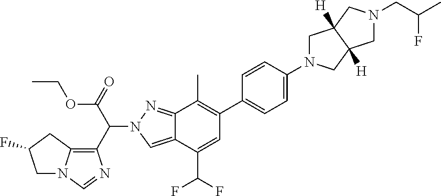 [SMILES: CCOC(=O)C(c1ncn2c1C[C@@H](F)C2)n1cc2c(C(F)F)cc(-c3ccc(N4C[C@H]5CN(CC(C)F)C[C@H]5C4)cc3)c(C)c2n1]

A mixture of Intermediate 1 (120 mg, 0.255 mmol, Eq: 1.0), [4-[(3aR,6aS)-2-(2-fluoropropyl)-1,3,3a,4,6,6a-hexahydropyrrolo[3,4-c]pyrrol-5-yl]phenyl]boronic acid (131 mg, 0.382 mmol, Eq: 1.5, 85% purity), Cs₂CO₃ (249 mg, 0.764 mmol, Eq: 3.0), Pd(dppf)Cl₂·CH₂Cl₂ (31 mg, 0.038 mmol, Eq: 0.15) in 1,4-dioxane (1.8 mL) was flushed with argon. The reaction mixture was stirred for 2 h at 100° C. and for 16 h at rt. The reaction mixture was diluted with EtOAc and water. The aqueous layer was back-extracted with EtOAc. The organic layers were washed with water and brine, dried over Na₂SO₄ and concentrated in vacuo. The crude material was adsorbed on Isolute HM-N and purified by flash chromatography (silica gel, 12 g, 0% to 5% MeOH in DCM) to give the title compound as a light brown foam (141 mg, 90% purity, 78% yield). m/z 639.3 [M+H]⁺, ESI pos.

### Step 7: 2-[6-[4-[(3aR,6aS)-2-(2-fluoropropyl)-1,3,3a,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide

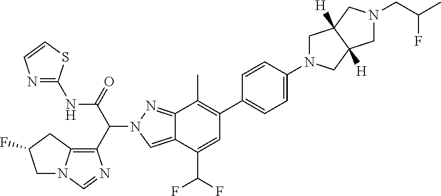 [SMILES: Cc1c(-c2ccc(N3C[C@H]4CN(CC(C)F)C[C@H]4C3)cc2)cc(C(F)F)c2cn(C(C(=O)Nc3nccs3)c3ncn4c3C[C@@H](F)C4)nc12]

By analogy with Example 8 Step 2, ethyl 2-[6-[4-[(3aR,6aS)-2-(2-fluoropropyl)-1,3,3a,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]acetate (139 mg, 0.196 mmol, Eq: 1.0, 90% purity) was initially treated with LiOH, and the resulting salt was reacted with 2-aminothiazole in the presence of HATU and DIPEA. Purification by flash chromatography (Si-amine, 12 g, 0% to 10% MeOH in EtOAc), followed repurification of product-containing fractions by flash chromatography (silica gel, 12 g, 0% to 10% MeOH in DCM) gave the title compound as an off-white solid (52 mg, 36% yield). m/z 693.4 [M+H]⁺, ESI pos.

### Example 30

### HTRF Phospho EGFR LRCS Assay (Cellular)

### Cell Line and Media

BaF3-LRCS cell line were obtained from Crownbio (San Diego, CA, USA). Cells were maintained at 37° C., 5% CO₂ in RPMI ATCC (Gibco 31870)+2 mM Glutamine+0.5 μg/ml Puromycin supplemented with 10% fetal bovine serum (FBS) (Gibco).

### Protocol

Cells are transferred as above to Greiner Bio-One, Nr. 784-08 micro-titerplate at 20000 cells/well in 12.5 μl of growth medium/well after the plates had been pre-filled with 12.5 nl of DMSO solutions of the to be tested compounds (in dose response) or DMSO only. After spinning the plates at 300×g for 30 seconds the cells were incubated for 4 hours at 37 C, 5% CO₂, 95% humidity. The cells were lysed by adding to the compound mix 4 μl/well of the supplemented lysis buffer (Cis-bio, Phospho-EGFR HTRF kit, 64EG1PEH), followed by incubation for 30 min at room temperature with shaking (400 rpm). The plates were then frozen and stored overnight at −80° C. On the next day and after thawing the plates, 4 μl of a mixture of anti-Phospho-EGFR Cryptate and of anti-Phospho-EGFR-d2 antibody solutions prepared in the supplied detection buffer was added to each well. The lidded plates were then incubated for 4 h at room temperature before reading the fluorescence emission at 616 and 665 nm using an Envision reader (Perkin Elmer). Data was analyzed in similar fashion as above using the normalized ratio of the 665 to 616 signals multiplied by 10000.

The results are shown in Table 1.

TABLE 8
BaF3 cellular HTRF Phospho EGFR LRCS assay data

| | | IC₅₀ |
|---|---|---|
| Exam. | Structure | (BaF3 LRCS) |
| 1 | 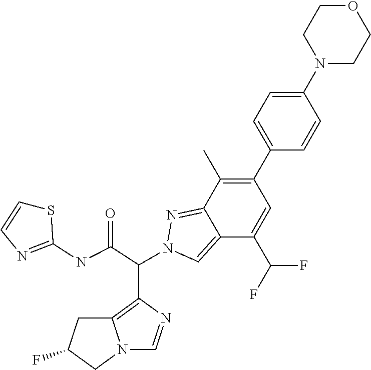 | 8nM |
| 2 | 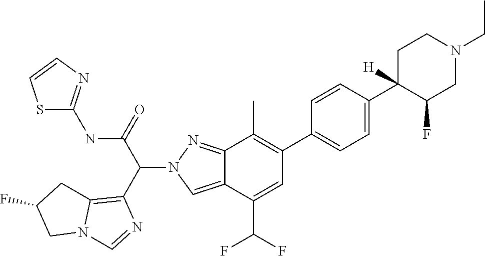 | 15nM |
| 3 | 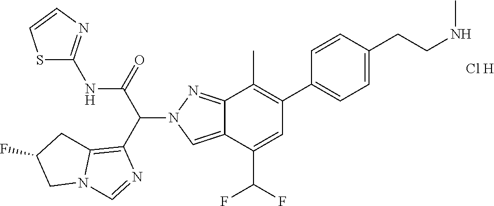 | 18nM |
| 4 | 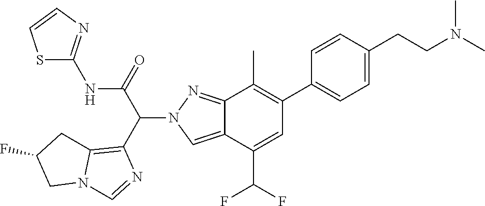 [SMILES: Cc1c(-c2ccc(CCN(C)C)cc2)cc(C(F)F)c2cn(C(C(=O)Nc3nccs3)c3ncn4c3C[C@@H](F)C4)nc12] | 11nM |
| 5 | 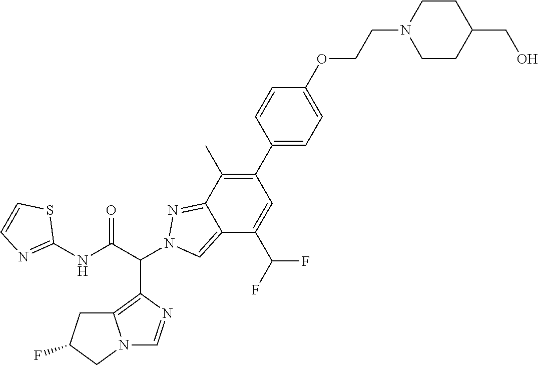 [SMILES: Cc1c(-c2ccc(OCCN3CCC(CO)CC3)cc2)cc(C(F)F)c2cn(C(C(=O)Nc3nccs3)c3ncn4c3C[C@@H](F)C4)nc12] | 19nM |
| 6 | 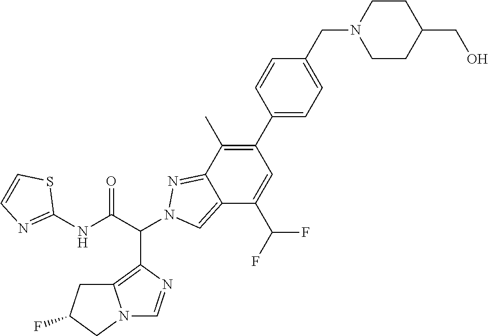 [SMILES: Cc1c(-c2ccc(CN3CCC(CO)CC3)cc2)cc(C(F)F)c2cn(C(C(=O)Nc3nccs3)c3ncn4c3C[C@@H](F)C4)nc12] | 16nM |
| 7 | 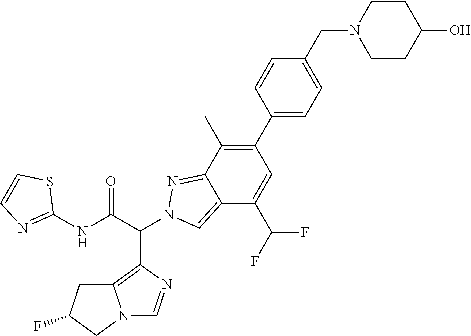 [SMILES: Cc1c(-c2ccc(CN3CCC(O)CC3)cc2)cc(C(F)F)c2cn(C(C(=O)Nc3nccs3)c3ncn4c3C[C@@H](F)C4)nc12] | 14nM |
| 8 | 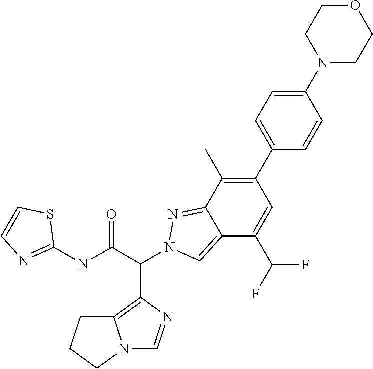 | 17nM |
| 9 | 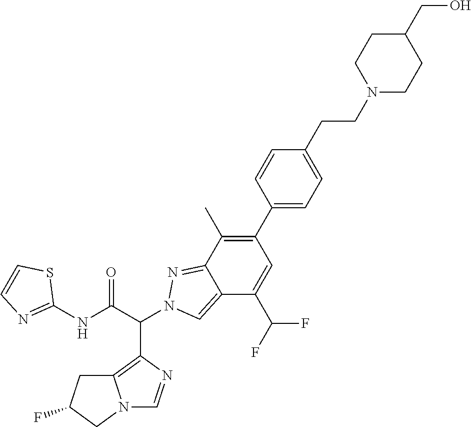 [SMILES: Cc1c(-c2ccc(CCN3CCC(CO)CC3)cc2)cc(C(F)F)c2cn(C(C(=O)Nc3nccs3)c3ncn4c3C[C@@H](F)C4)nc12] | 27nM |
| 10 | 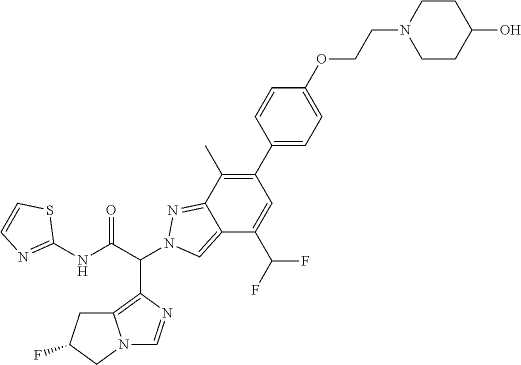 [SMILES: Cc1c(-c2ccc(OCCN3CCC(O)CC3)cc2)cc(C(F)F)c2cn(C(C(=O)Nc3nccs3)c3ncn4c3C[C@@H](F)C4)nc12] | 17nM |
| 11 | 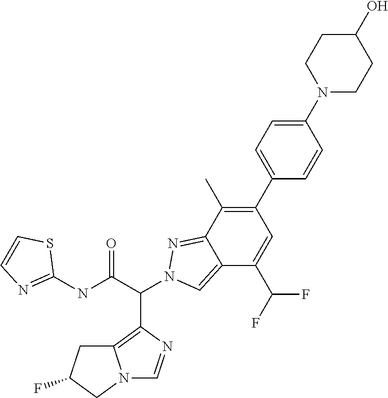 | 10nM |
| 12 | 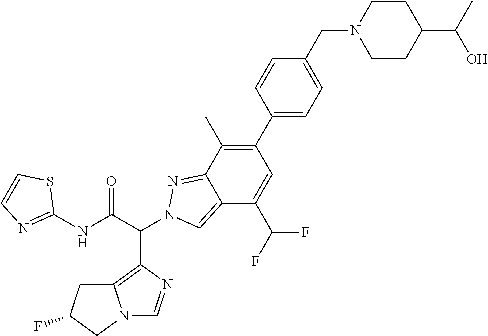 [SMILES: Cc1c(-c2ccc(CN3CCC(C(C)O)CC3)cc2)cc(C(F)F)c2cn(C(C(=O)Nc3nccs3)c3ncn4c3C[C@@H](F)C4)nc12] | 18nM |
| 13 | 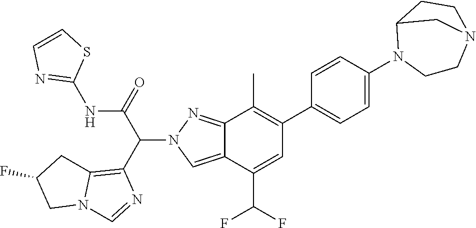 [SMILES: Cc1c(-c2ccc(N3CCN4CCC3C4)cc2)cc(C(F)F)c2cn(C(C(=O)Nc3nccs3)c3ncn4c3C[C@@H](F)C4)nc12] | 17nM |
| 14 | 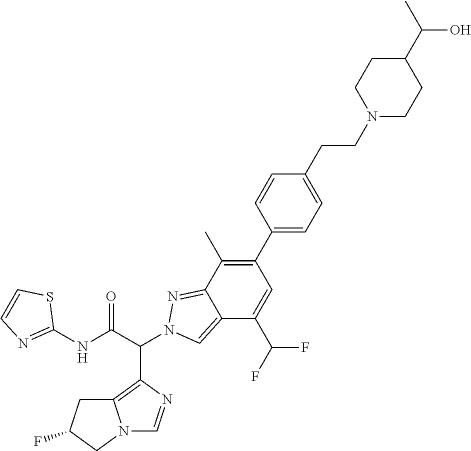 [SMILES: Cc1c(-c2ccc(CCN3CCC(C(C)O)CC3)cc2)cc(C(F)F)c2cn(C(C(=O)Nc3nccs3)c3ncn4c3C[C@@H](F)C4)nc12] | 16nM |
| 15 | 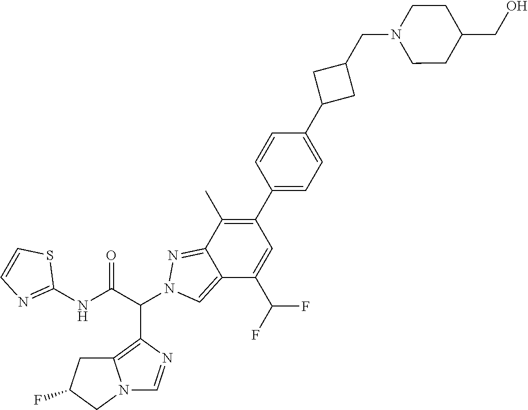 [SMILES: Cc1c(-c2ccc(C3CC(CN4CCC(CO)CC4)C3)cc2)cc(C(F)F)c2cn(C(C(=O)Nc3nccs3)c3ncn4c3C[C@@H](F)C4)nc12] | 24nM |
| 16 | 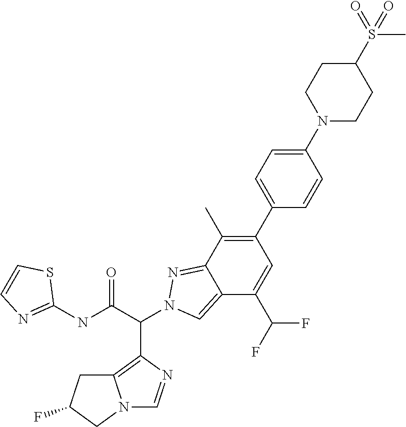 | 16nM |
| 17 | 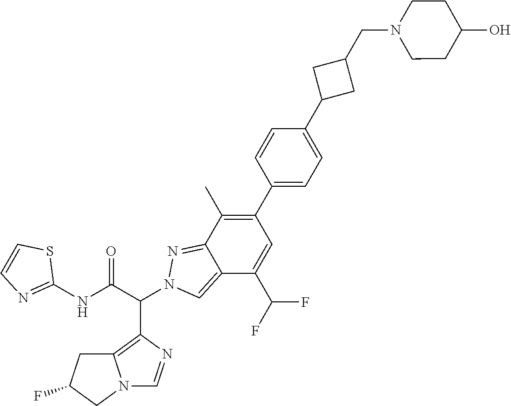 [SMILES: Cc1c(-c2ccc(C3CC(CN4CCC(O)CC4)C3)cc2)cc(C(F)F)c2cn(C(C(=O)Nc3nccs3)c3ncn4c3C[C@@H](F)C4)nc12] | 30nM |
| 18 | 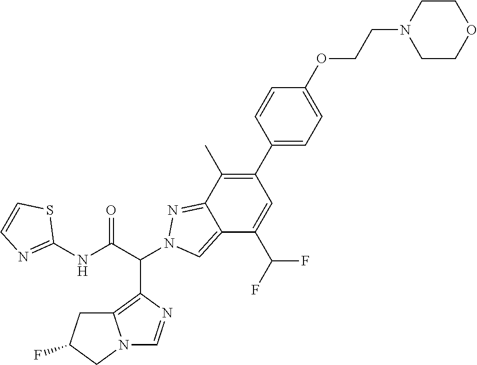 [SMILES: Cc1c(-c2ccc(OCCN3CCOCC3)cc2)cc(C(F)F)c2cn(C(C(=O)Nc3nccs3)c3ncn4c3C[C@@H](F)C4)nc12] | 28nM |
| 19 | 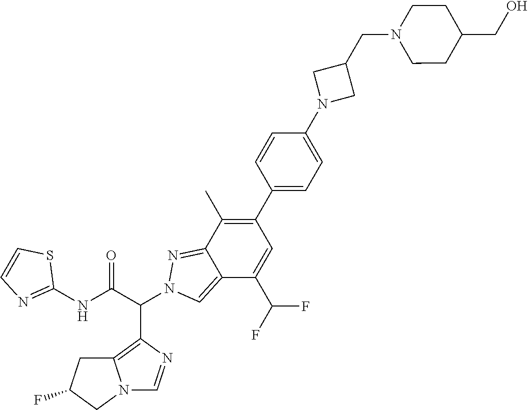 [SMILES: Cc1c(-c2ccc(N3CC(CN4CCC(CO)CC4)C3)cc2)cc(C(F)F)c2cn(C(C(=O)Nc3nccs3)c3ncn4c3C[C@@H](F)C4)nc12] | 16nM |
| 20 | 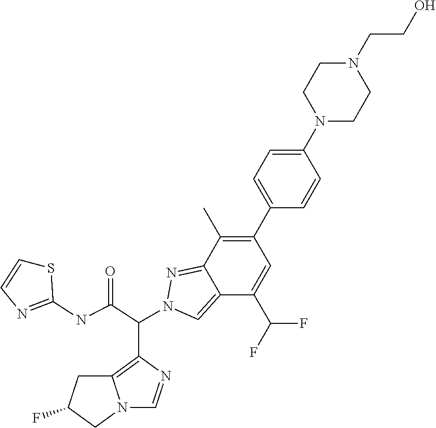 | 12nM |
| 21 | 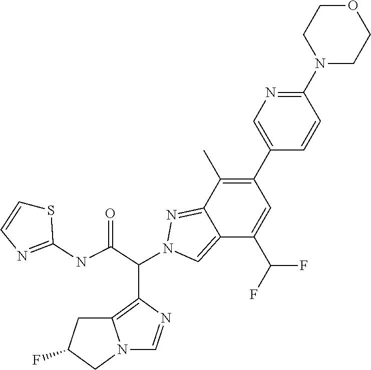 | 12nM |
| 22 | 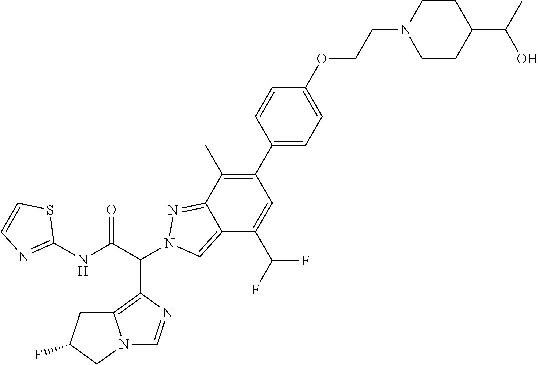 [SMILES: Cc1c(-c2ccc(OCCN3CCC(C(C)O)CC3)cc2)cc(C(F)F)c2cn(C(C(=O)Nc3nccs3)c3ncn4c3C[C@@H](F)C4)nc12] | 30nM |
| 23 | 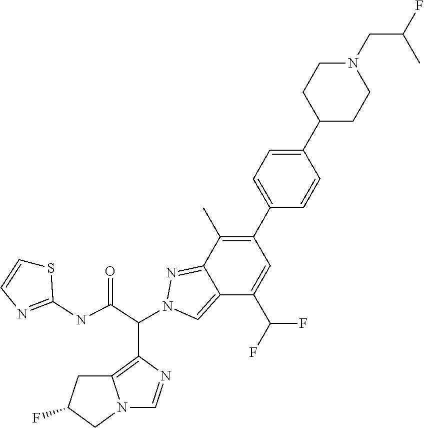 | 21nM |
| 24 | 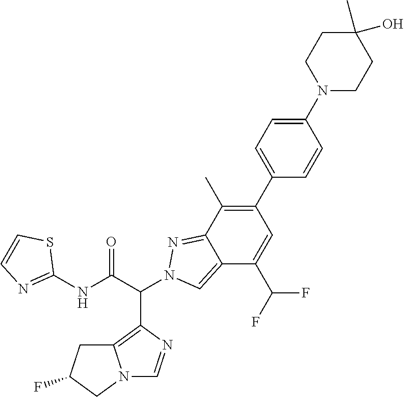 [SMILES: Cc1c(-c2ccc(N3CCC(C)(O)CC3)cc2)cc(C(F)F)c2cn(C(C(=O)Nc3nccs3)c3ncn4c3C[C@@H](F)C4)nc12] | 25nM |
| 25 | 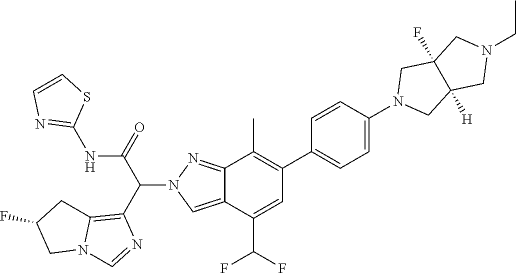 [SMILES: CCN1C[C@H]2CN(c3ccc(-c4cc(C(F)F)c5cn(C(C(=O)Nc6nccs6)c6ncn7c6C[C@@H](F)C7)nc5c4C)cc3)C[C@@]2(F)C1] | 29nM |
| 26 | 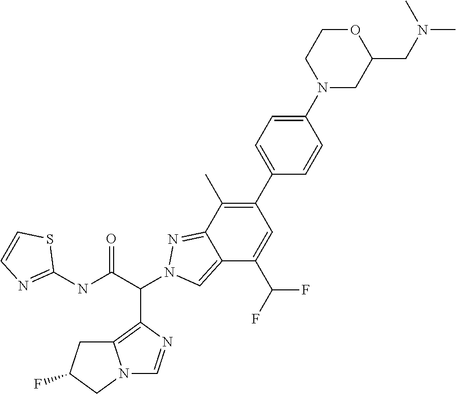 [SMILES: Cc1c(-c2ccc(N3CCOC(CN(C)C)C3)cc2)cc(C(F)F)c2cn(C(C(=O)Nc3nccs3)c3ncn4c3C[C@@H](F)C4)nc12] | 78nM |
| 27 | 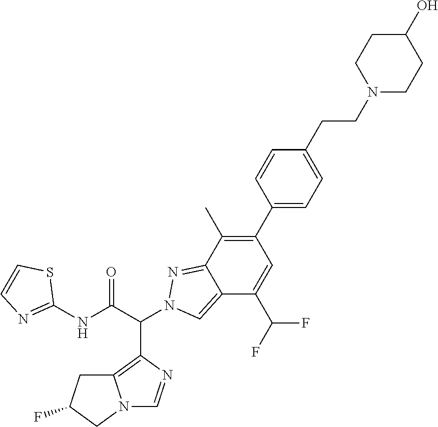 [SMILES: Cc1c(-c2ccc(CCN3CCC(O)CC3)cc2)cc(C(F)F)c2cn(C(C(=O)Nc3nccs3)c3ncn4c3C[C@@H](F)C4)nc12] | 28nM |
| 28 | 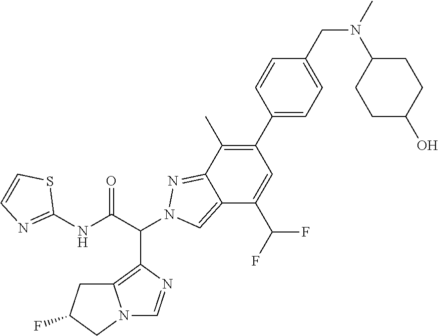 [SMILES: Cc1c(-c2ccc(CN(C)C3CCC(O)CC3)cc2)cc(C(F)F)c2cn(C(C(=O)Nc3nccs3)c3ncn4c3C[C@@H](F)C4)nc12] | 34nM |
| 29 | 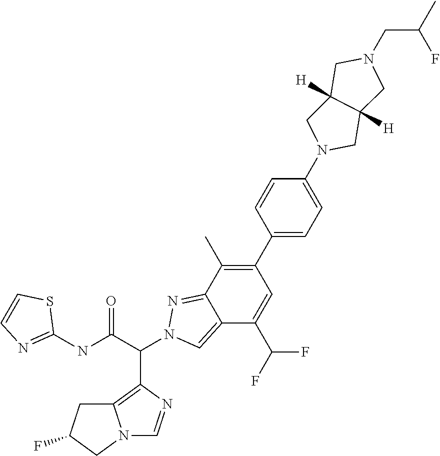 [SMILES: Cc1c(-c2ccc(N3C[C@H]4CN(CC(C)F)C[C@H]4C3)cc2)cc(C(F)F)c2cn(C(C(=O)Nc3nccs3)c3ncn4c3C[C@@H](F)C4)nc12] | 28nM |

1. A compound of formula (I) 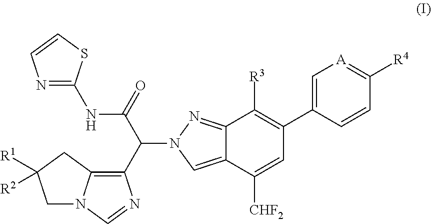
wherein
A is —N— or —CH—;
R¹ and R² are independently selected from halogen and hydrogen;
R³ is alkyl; and
R⁴ is selected from heterocycloalkyl, heterocycloalkylalkoxy, alkylaminoalkyl, dialkylaminoalkyl, hydroxyalkylheterocycloalkylalkoxy, hydroxyalkylheterocycloalkylalkyl, hydroxyheterocycloalkylalkyl, hydroxyheterocycloalkylalkoxy, (alkyl)(halo)heterocycloalkyl, hydroxyheterocycloalkyl, hydroxyalkylheterocycloalkylalkylcycloalkyl, alkylsulfonylheterocycloalkyl, hydroxyheterocycloalkylalkylcycloalkyl, hydroxyalkylheterocycloalkylalkylheterocycloalkyl, hydroxyalkylheterocycloalkyl, haloalkylheterocycloalkyl, (hydroxy)(alkyl)heterocycloalkyl, hydroxycycloalkyl(alkylamino)alkyl, dialkylaminoalkylheterocycloalkyl and heterocycloalkylalkyl;
or a pharmaceutically acceptable salt thereof.

2. A compound according to claim 1, wherein A is —CH—.

3. A compound according to claim 1 or 2, wherein R¹ and R² are independently selected from fluoro and hydrogen.

4. A compound according to any one of claims 1 to 3, wherein R³ is methyl.

5. A compound according to any one of claims 1 to 4, wherein R⁴ is selected from morpholinyl, (ethyl)(fluoro)piperidinyl, methylaminoethyl, dimethylaminoethyl, hydroxymethylpiperidinylethoxy, hydroxyethylpiperidinylethoxy, hydroxymethylpiperidinylmethyl, hydroxymethylpiperidinylethyl, hydroxyethylpiperidinylmethyl, hydroxyethylpiperidinylethyl, hydroxypiperidinylmethyl, hydroxypiperidinylethyl, hydroxypiperidinylethoxy, hydroxypiperidinyl, 1,4-diazabicyclo[3.2.1]octan-4-yl, hydroxymethylpiperidinylmethylcyclobutyl, methylsulfonylpiperidinyl, hydroxypiperidinylmethylcyclobutyl, morpholinylethoxy, hydroxymethylpiperidinylmethylazetidinyl, hydroxyethylpiperazinyl, fluoropropylpiperidinyl, (hydroxy)(methyl)piperidinyl, ethyl(fluorohexahydropyrrolo[3,4-c]pyrrolyl), dimethylaminomethylmorpholinyl, hydroxycyclohexyl(methylamino)methyl and fluoropropyl(hexahydropyrrolo[3,4-c]pyrrolyl).

6. A compound according to any one of claims 1 to 4, wherein R⁴ is selected from heterocycloalkyl, (dialkylamino)alkyl, hydroxy(heterocycloalkyl)alkyl, hydroxy(heterocycloalkyl) and hydroxyalkyl(heterocycloalkyl).

7. A compound according to any one of claims 1 to 6, wherein R⁴ is selected from morpholinyl, (dimethylamino)ethyl, hydroxy(piperidinyl)methyl, hydroxy(piperidinyl) and hydroxyethyl (piperazinyl.

8. A compound according to any one of claims 1 to 7 selected from
2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(3 S,4S)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(3R,4R)-1-ethyl-3-fluoro-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-[4-[2-(methylamino)ethyl]phenyl]indazol-2-yl]-2-[(6R)-6-fluoro-6,7-dihydro-5H-pyrrolo[1,2-c]imidazol-1-yl]-N-thiazol-2-yl-acetamide hydrochloride;
2-[4-(difluoromethyl)-6-[4-[2-(dimethylamino)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[4-(hydroxymethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[[4-(hydroxymethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(4-morpholinophenyl)indazol-2-yl]-2-(6,7-dihydro-5H-pyrrolo[1,2-c]imidazol-1-yl)-N-thiazol-2-yl-acetamide;
2-[4-(difluoromethyl)-6-[4-[2-[4-(hydroxymethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-(4-hydroxy-1-piperidyl)ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-(4-hydroxy-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[[4-(1-hydroxyethyl)-1-piperidyl]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[6-[4-(1,4-diazabicyclo[3.2.1]octan-4-yl)phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[4-(1-hydroxyethyl)-1-piperidyl]ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[3-[[4-(hydroxymethyl)-1-piperidyl]methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-[4-(4-methylsulfonyl-1-piperidyl)phenyl]indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[3-[(4-hydroxy-1-piperidyl)methyl]cyclobutyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-[4-(2-morpholinoethoxy)phenyl]indazol-2-yl]-2-[(6R)-6-fluoro-6,7-dihydro-5H-pyrrolo[1,2-c]imidazol-1-yl]-N-thiazol-2-yl-acetamide;
2-(4-(difluoromethyl)-6-(4-(3-((4-(hydroxymethyl)piperidin-1-yl)methyl)azetidin-1-yl)phenyl)-7-methyl-2H-indazol-2-yl)-2-((R)-6-fluoro-6,7-dihydro-5H-pyrrolo[1,2-c]imidazol-1-yl)-N-(thiazol-2-yl)acetamide;
2-[4-(difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[4-(1-hydroxyethyl)-1-piperidyl]ethoxy]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[1-(2-fluoropropyl)-4-piperidyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-(4-hydroxy-4-methyl-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[6-[4-[(3 aR,6aS)-2-ethyl-3a-fluoro-3,4,6,6a-tetrahydro-1H-pyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-[(dimethylamino)methyl]morpholin-4-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-(4-hydroxy-1-piperidyl)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[[(4-hydroxycyclohexyl)-methyl-amino]methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide; and
2-[6-[4-[(3aS,6aR)-2-(2-fluoropropyl)-1,3,3a,4,6,6a-hexahydropyrrolo[3,4-c]pyrrol-5-yl]phenyl]-4-(difluoromethyl)-7-methyl-indazol-2-yl]-2-[(6R)-6-fluoro-6,7-dihydro-5H-pyrrolo[1,2-c]imidazol-1-yl]-N-thiazol-2-yl-acetamide;
or a pharmaceutically acceptable salt thereof.

9. A compound according to any one of claims 1 to 8 selected from
2-[4-(difluoromethyl)-7-methyl-6-(4-morpholinophenyl)indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[2-(dimethylamino)ethyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[(4-hydroxy-1-piperidyl)methyl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-(4-hydroxy-1-piperidyl)phenyl]-7-methyl-indazol-2-yl]-2-[(6R)-6-fluoro-6,7-dihydro-5H-pyrrolo[1,2-c]imidazol-1-yl]-N-thiazol-2-yl-acetamide;
2-[4-(difluoromethyl)-6-[4-[4-(2-hydroxyethyl)piperazin-1-yl]phenyl]-7-methyl-indazol-2-yl]-2-[(6R)-6-fluoro-6,7-dihydro-5H-pyrrolo[1,2-c]imidazol-1-yl]-N-thiazol-2-yl-acetamide; and
2-[4-(difluoromethyl)-7-methyl-6-(6-morpholino-3-pyridyl)indazol-2-yl]-2-[(6R)-6-fluoro-6,7-dihydro-5H-pyrrolo[1,2-c]imidazol-1-yl]-N-thiazol-2-yl-acetamide;
or a pharmaceutically acceptable salt thereof.

10. A process for the preparation of a compound according to any one of claims 1 to 9, comprising the following steps:
(a) the reaction of a compound of formula (B1) 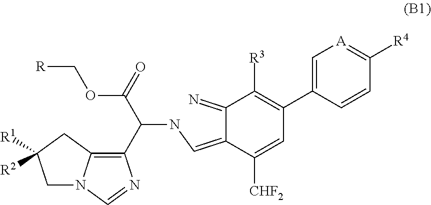
in a suitable solvent and in presence of a base, to arrive at a compound of formula (B2) 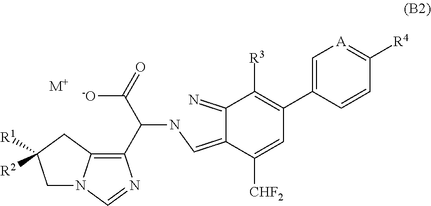
wherein M⁺ is Na⁺, Li⁺ or a protonated base;
(b) the reaction of the compound of formula (B2) in presence of an acid to yield a compound of formula (B3) 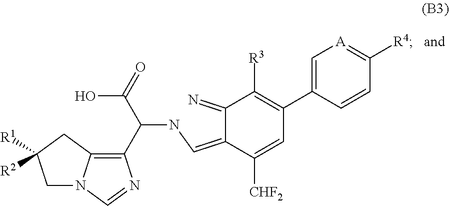
(c) the reaction of the compound of formula (B3) with a compound of formula (B4) 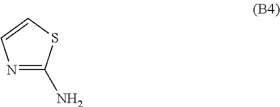
in the presence of a coupling agent and a base;
wherein A, R¹, R², R³ and R⁴ are as defined in any one of claims 1 to 9, and wherein R is hydrogen or alkyl.

11. A compound according to any one of claims 1 to 9, when manufactured according to a process of claim 10.

12. A compound according to any one of claims 1 to 9 for use as therapeutically active substance.

13. A pharmaceutical composition comprising a compound according to any one of claims 1 to 9 and a therapeutically inert carrier.

14. A compound according to anyone of claims 1 to 9 for use in the treatment or prophylaxis of cancer, in particular non-small cell lung cancer.

15. The use of a compound according to any one of claims 1 to 9 for the treatment or prophylaxis of cancer, in particular non-small cell lung cancer.

16. The use of a compound according to any one of claims 1 to 9 for the preparation of a medicament for the treatment or prophylaxis of cancer, in particular non-small cell lung cancer.

17. A method for the treatment or prophylaxis of cancer, in particular non-small cell lung cancer, which method comprises administering an effective amount of a compound as defined in any one of claims 1 to 9 to a patient in need thereof.

18. The invention as hereinbefore described.
